(19) United States
(12) Patent Application Publication
(10) Pub. No.: US 2025/0195666 A1
(43) Pub. Date: Jun. 19, 2025

(54) MIR-142 COMPOUNDS AND USES THEREOF

(71) Applicant: City of Hope, Duarte, CA (US)

(72) Inventors: Bin Zhang, Duarte, CA (US); Guido Marcucci, Duarte, CA (US)

(21) Appl. No.: 18/561,953

(22) PCT Filed: May 18, 2022
(86) PCT No.: PCT/US2022/029883

§ 371 Date: Nov. 17, 2023

Related U.S. Application Data
(60) Provisional application No. 63/191,011, filed on May 20, 2021.

(51) Int. Cl. A61K 47/54; A61P 35/02; C12N 15/113; C12N 15/117

(52) U.S. Cl. CPC A61K 47/549; A61K 47/548; A61P 35/02; C12N 15/1135; C12N 15/117; C12N 2310/141; C12N 2310/17; C12N 2320/32

(57) ABSTRACT

The disclosure provides, inter alia, compounds comprising Toll-like receptor 9-binding nucleic acid sequences and nucleic acid sequences comprising a microRNA-142 passenger strand sequence hybridized to a microRNA-142 guide strand sequence; pharmaceutical compositions comprising the compounds; and the use of the compounds and pharmaceutical compositions to treat myeloid leukemia.

### CROSS-REFERENCE TO RELATED APPLICATIONS

This application claims the benefit of priority to U.S. Application No. 63/191,011 filed May 20, 2021, the disclosure of which is incorporated by reference herein in its entirety.

### REFERENCE TO A “SEQUENCE LISTING,” A TABLE, OR A COMPUTER PROGRAM LISTING APPENDIX SUBMITTED AS AN ASCII FILE

The Sequence Listing written in file .txt, created, bytes, machine format IBM-PC, MS Windows operating system, is hereby incorporated by reference.

### BACKGROUND

Acute myeloid leukemia (AML) is a hematopoietic malignancy characterized by acquired mutations and aberrant expression of genes causing cell differentiation arrest and accumulation of hematopoietic stem and progenitor cells (HSPCs) and partially differentiated “bulk” blasts that leads to bone marrow (BM) failure. Despite our understanding of the pathogenesis of this disease and emerging novel molecular targeted therapeutics, the overall outcome of these patients remains poor. While prognostic assessment and risk-stratification therapies are based mainly on cytogenetic and molecular aberrations harbored by the leukemic blasts, consideration of additional clinical features is key for proper selection of therapeutic programs. In fact, patient with therapy-related disease (t-AML; i.e., prior exposure to radiation or chemotherapy for other unrelated cancers) or secondary (s) AML (i.e., antecedent clonal hematopoietic disorders (ACHD) including myelodysplastic syndrome (MDS), MDS/myeloproliferative neoplasm (MDS/MPN; i.e., chronic myelomonocytic leukemia)) and MPN (i.e., essential thrombocythemia, polycythemia vera, myelofibrosis)) have a significantly worse outcome than patients with de novo AML. Similarly patients with chronic myelogenous leukemia (CML) that has progressed from chronic phase (CP) to blast crisis (BC) have very poor prognosis. Unfortunately, sAML (secondary acute myeloid leukemia) and BC CML (blast crisis chronic myelogenous leukemia) patients not only respond poorly to the current therapies but they are often excluded from promising clinical trials. Thus, the availability of novel and more effective treatments is a true unmet need for these patients. The disclosure is directed to this, as well as other, important ends.

### BRIEF SUMMARY

Provided herein are compounds comprising a CpG oligodeoxynucleotide (ODN) covalently bonded to a hybridized nucleic acid sequence, wherein the hybridized nucleic acid sequence comprises a miR-142 passenger strand sequence hybridized to a miR-142 guide strand sequence. This disclosure provides pharmaceutical compositions comprising the compounds and a pharmaceutically acceptable excipient.

Provided herein are methods of treating myeloid leukemia by administering to a patient an effective amount of a compound comprising a CpG oligodeoxynucleotide (ODN) covalently bonded to a hybridized nucleic acid sequence, wherein the hybridized nucleic acid sequence comprises a miR-142 passenger strand sequence hybridized to a miR-142 guide strand sequence. In embodiments, the myeloid leukemia is chronic myeloid leukemia, chronic phase of chronic myeloid leukemia, accelerated phase of chronic myeloid leukemia, blast phase of chronic myeloid leukemia, acute myeloid leukemia, secondary acute myeloid leukemia, secondary acute myeloid leukemia related to therapy, secondary acute myeloid leukemia related to an antecedent hematologic disorder (e.g., myelodysplastic syndrome, myeloproliferative neoplasm, or myelodysplastic syndrome/myeloproliferative neoplasm overlap syndrome). In embodiments, the patient has reduced levels of miR-142 relative to a control. In embodiments, the methods further comprise administering to the patient an effective amount of a tyrosine kinase inhibitor.

Provided herein are methods of treating myelodysplastic syndrome, myeloproliferative neoplasm, or myelodysplastic syndrome/myeloproliferative neoplasm overlap syndrome by administering to a patient an effective amount of a compound comprising a CpG oligodeoxynucleotide (ODN) covalently bonded to a hybridized nucleic acid sequence, wherein the hybridized nucleic acid sequence comprises a miR-142 passenger strand sequence hybridized to a miR-142 guide strand sequence. In embodiments, the patient has reduced levels of miR-142 relative to a control. In embodiments, the methods further comprise administering to the patient an effective amount of a tyrosine kinase inhibitor.

Provided herein are methods of preventing or delaying the progression of an antecedent clonal hematopoietic disorder in a patient in need thereof by administering to a patient an effective amount of a compound comprising a CpG oligodeoxynucleotide (ODN) covalently bonded to a hybridized nucleic acid sequence, wherein the hybridized nucleic acid sequence comprises a miR-142 passenger strand sequence hybridized to a miR-142 guide strand sequence; wherein the patient has the antecedent clonal hematopoietic disorder. In embodiments, the methods are for preventing or delaying the progression of the antecedent clonal hematopoietic disorder to acute myeloid leukemia. In embodiments, the methods are for preventing or delaying the progression of the antecedent clonal hematopoietic disorder to blast crisis chronic myelogenous leukemia. In embodiments, the patient has reduced levels of miR-142 relative to a control. In embodiments, the methods further comprise administering to the patient an effective amount of a tyrosine kinase inhibitor.

These and other embodiments of the disclosure are described herein.

### BRIEF DESCRIPTION OF THE DRAWINGS

FIGS. 1A-1D. Increased LSK and GMP and reduced LT-HSC and MEP in the BM of miR-142−/− mice.

FIGS. 2A-2B. BCR-ABL levels are associated with CP CML morbidity but not with BC transformation.

FIGS. 3A-3C. Lower miR-142 levels in CD34+ and CD34+CD38− cells from BC CML patients than their counterparts from CP CML patients.

FIGS. 4A-4B. Leukemic blasts in PB and BM from miR-142 KO BCR-ABL mice but not in miR-142 wt BCR-ABL mice.

FIGS. 5A-5B. miR-142 dosage dependent BC transformation.

FIGS. 6A-6B. BC CML features were recapitulated in the recipient mice engrafted with LSKs from miR142−/− BCR-ABL mice.

FIGS. 7A-7B: GSEA of differentially expressed pathways in miR-142 KO vs. miR-142 wt BCR-ABL+LSKs.

FIG. 8. CpG-M-miR-142 reduced CPT1A/B, FAO and OCR in LSC-enriched CD34+CD38− AML blasts.

FIG. 9. CpG-M-miR-142 reduced target gene expression and cell growth and increased NIL-induced apoptosis in miR-142 KO LSCs.

FIGS. 10A-10C: In vivo treatment with CpG-M-miR-142 reduced BC CML burden and miR-142 target gene expression in miR-142 KO CML mice.

FIGS. 11A-11B. Q-RT-PCR showed lower levels of miR142-3p in MNCs from AML patients (n=238) than those from normal individuals (n=17) and lower in FLT3ITD+vs FLT3-ITD-AM MNCs.

FIGS. 12A-12B. miR-142 KO transformed Flt3^{ITDITD}-induced MPN to AML.

FIGS. 13A-13B. Transplant of BM cells from diseased miR-142 KO Flt3^{ITDITD} mice recapitulated AML in recipient mice.

FIG. 14. miR-142 KO promotes more aggressive AML development.

FIGS. 15A-15B: Structure of CpG-miR142-3p (FIG. 15A) and CpG-miR142-5p (FIG. 15B); where each “o” is:

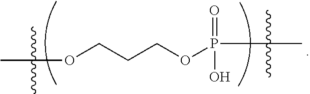

FIG. 16: miR-142 levels are lower in human T cells from AML patients vs those from healthy donors (left) and in mouse T cells from Mll^{PTD/WT}Flt3^{ITD/ITD} AML mice vs those from normal wt mice (right), as analyzed by Q-RT-PCR.

FIGS. 17A-17B: Both mouse (FIG. 17A) and human (FIG. 17B) T cells with miR-142 deficit showed increased baseline apoptosis, reduced cell cycling, cell growth and cytokine production compared to miR-142 wt T cells.

FIG. 18: Uptake of M-miR-142 conjugated with Cy3 in LSKs treated in vitro for four hours (left) and in LSKs from CML mice treated in vivo for four hours (right).

FIGS. 19A-19D: miR-142 KO BCR-ABL mice were treated with M-miR-142 (20 mg/kg/day, iv) or scramble RNA (SCR) at day 2 after BCR-ABL induction for four weeks (FIG. 19A) and miR-142 expression in BM cells was measured by Q-RT-PCR (FIG. 19B) and white blood cell (WBC) counts, LSK in periphery blood (PB), leukemic blasts in BM and survival of the treated mice were evaluated (FIG. 19C). BM cells from the treated mice were transplanted into recipient mice and WBC counts, engraftment in PB and survival of the 2^{nd} recipient mice were monitored (FIG. 19D).

FIGS. 20A-20C: CD45.2 BM cells from diseased miR-142 KO BCR-ABL mice (BC CML, BCR-ABL induced for four weeks) were transplanted into CD45.1 recipient mice. At day 15 after transplantation, these BC CML mice were treated with M-miR-142 (20 mg/kg/day, iv) or SCR for three weeks (FIG. 20A). WBC counts, engraftment in PB, BM and spleen, and survival of the treated mice were evaluated (FIG. 20B). BM cells from these treated mice were transplanted into 2^{nd} recipient mice and WBC counts, engraftment in PB and survival of the 2^{nd} recipient mice were shown (FIG. 20C).

FIGS. 21A-21C: CD45.2 BM cells from diseased miR-142 KO BCR-ABL mice (BC CML, BCR-ABL induced for four weeks) were transplanted into CD45.1 recipient mice. At day 15 after transplantation, these BC CML mice were treated with M-miR-142 (20 mg/kg/day, iv)+nilotinib (NIL, 100 mg/kg/day, oral gavage) or SCR+NIL for three weeks (FIG. 21A). WBC counts, engraftment in PB, and survival of the treated mice were evaluated (FIG. 21B). BM cells from these treated mice were transplanted into 2^{nd} recipient mice and engraftment in PB and survival of the 2^{nd} recipient mice were shown (FIG. 21C).

FIGS. 22A-22C: A cohort of patient derived xenograft (PDX) were generated by transplanting human CD34+ cells from BC CML patient 22-1025. When >5% human engraftment rates were detected in PB at day 15 after transplantation, these mice were treated with M-miR-142 (20 mg/kg/day, iv) or SCR for three weeks (FIG. 22A). Levels of miR-142 in BM and survival of the treated mice were shown (FIG. 22B). BM cells from these treated mice were transplanted into 2^{nd} recipient mice and engraftment in PB and survival of the 2^{nd} recipient mice were shown (FIG. 22C).

FIGS. 23A-23C: A cohort of patient derived xenograft (PDX) were generated by transplanting human CD34+ cells from BC CML patient 22-1025. When >5% human engraftment rates were detected in PB at day 15 after transplantation, these mice were treated with M-miR-142 (20 mg/kg/day, iv)+nilotinib (NIL, 100 mg/kg/day, oral gavage) or SCR+NIL for three weeks (FIG. 23A). Engraftment in PB, BM and spleen, spleen weight, and survival of the treated mice are shown (FIG. 23B). BM cells from these treated mice were transplanted into recipient mice and engraftment rates in the PB of the 2^{nd} recipient mice are shown (survival is pending) (FIG. 23C).

FIGS. 24A-24B: A cohort of patient derived xenograft (PDX) were generated by transplanting human CD34+ cells from BC CML patient 22-1025. When >5% human engraftment rates were detected in PB at day 15 after transplantation, these mice were injected with one dose of autologous T cells (collected from the same patient and expanded in vitro for 14 days, 1×10⁶/mouse) and then treated with M-miR-142 (−3p, 20 mg/kg/day; −5p, 10 mg/kg/day; iv) or SCR for three weeks (FIG. 24A). Engraftment in PB, and survival of the treated mice are shown (FIG. 24B).

### DETAILED DESCRIPTION

### Definitions

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices and materials similar or equivalent to those described herein can be used in the practice of this disclosure. The following definitions are provided to facilitate understanding of certain terms used frequently herein and are not meant to limit the scope of the present disclosure.

The term “acute myeloid leukemia” or “AML” is a biologically heterogeneous disease that can be classified into two distinct categories: (1) de novo AML that arises in the absence of an identified exposure or prodromal stem cell disorder, and (2) secondary AML (sAML). Secondary AML can be further divided into therapy-related AML (t-AML) due to previous exposure to leukemogenic therapies including chemotherapy and radiotherapy, or AML evolving from antecedent hematologic disorder (AHD-AML) including myelodysplastic syndrome (MDS), myeloproliferative neoplasm (MPN), or aplastic anemia (AA). “AML evolving from antecedent hematologic disorder” is alternatively referred to herein as “antecedent hematologic disorder-related AML.” In embodiments, the antecedent hematologic disorder is myelodysplastic syndrome (MDS), myeloproliferative neoplasm (MPN), or myelodysplastic syndrome/myeloproliferative neoplasm overlap syndrome (MDS/MPN).

The term “chronic myeloid leukemia” or “CML” is a hematopoietic malignancy characterized by an increase and unregulated growth of predominantly myeloid cells in the bone marrow, and their accumulation in the blood. A hallmark of CML is the Philadelphia (Ph) chromosome resulting from a reciprocal translocation between the long arms of chromosomes 9 and 22. This chromosomal translocation leads to expression of BCR-ABL, an oncogenic fusion-protein with a constitutively activated ABL tyrosine kinase. BCR-ABL can transform myeloid progenitor cells and drives the development of 95% of CML cases. BCR-ABL promotes leukemogenesis by activating downstream signaling proteins that increase cell survival and proliferation. These pathways include, but are not limited to, the RAS/mitogen-activated protein kinase (RAF/MEK/ERK), phosphatidylinositol 3-kinase/AKT (PI3K/AKT), and JAK/STAT signaling cascades.

The term “chronic phase” refers to the chronic phase of CML where the patient may or may not have symptoms, the patient has an increased number of white blood cells, and the patient usually responds to standard treatment. In chronic phase CML, less than 10% of the blood cells in the patient's bone marrow are immature blasts.

The term “accelerated phase” refers to an advanced phase of CML where the patient will likely have symptoms, the patient will have increased numbers of white blood cells in the blood stream (e.g., more than 20% basophils), and the patient will have from 10% and 30% immature blasts in the bone marrow.

The term “blast phase” or “blast crisis phase” refers to an advanced phase of CML that behaves like acute myeloid leukemia. Patients with blast phase CML may have anemia, high white blood cell counts, very high or very low platelet counts, CML cells with new chromosome abnormalities (e.g., in addition to the Ph chromosomes), and blast cells that have spread outside the blood and/or bone marrow into other tissues and organs. In blast phase CML, more than 30% of the blood cells in the in the patient's bone marrow or blood will be immature blast cells.

A “microRNA,” “microRNA nucleic acid sequence,” “miR,” “miRNA” as used herein, refers to a nucleic acid that functions in RNA silencing and post-transcriptional regulation of gene expression. The term includes all forms of a miRNA, such as the pri-, pre-, and mature forms of the miRNA. In embodiments, microRNAs (miRNAs) are short (20-24 nt) non-coding RNAs that are involved in post-transcriptional regulation of gene expression in multicellular organisms by affecting both the stability and translation of mRNAs. miRNAs are transcribed by RNA polymerase II as part of capped and polyadenylated primary transcripts (pri-miRNAs) that can be either protein-coding or non-coding. The primary transcript is cleaved by the Drosha ribonuclease III enzyme to produce an approximately 70-nt stem-loop precursor miRNA (pre-miRNA), which is further cleaved by the cytoplasmic Dicer ribonuclease to generate the mature miRNA and antisense miRNA star (miRNA*) products. The mature miRNA is incorporated into a RNA-induced silencing complex (RISC), which recognizes target mRNAs through imperfect base pairing with the miRNA and most commonly results in translational inhibition or destabilization of the target mRNA. In embodiments, a miRNA nucleic acid sequence is about 2 to 50 nucleotides in length. In embodiments, a miRNA nucleic acid sequence is about 10 to 30 nucleotides in length. In embodiments, a miRNA nucleic acid sequence is about 15 to 25 nucleotides in length.

The term “microRNA-mimic (miRNA-mimic)” or “miRNA-mimic nucleic acid sequence” is used according to its plain and ordinary meaning and refers to single, double or triple stranded oligonucleotide that is capable of effecting a biological function similar to a microRNA. In embodiments, miRNA-mimic may be non-natural double-stranded miR-like RNA fragments. Such an RNA fragment may be designed to have its 5′-end bearing a partially complementary motif to the selected sequence in the 3′UTR unique to the target gene. Once introduced into cells, this RNA fragment, may mimic an endogenous miRNA, bind specifically to its target gene and produce posttranscriptional repression, more specifically translational inhibition, of the gene. Unlike endogenous miRNAs, miRNA-mimics may act in a gene-specific fashion. In embodiments, the miRNA-mimic is a double stranded oligomer of 10 to 30 bases. In embodiments, the miRNA-mimic is a triple stranded oligomer of 10-30 bases. In embodiments, the miRNA-mimic has a 2′-chemical modification. In embodiments, the miRNA-mimic has serum stability-enhancing chemical modification, e.g., a phosphorothioate internucleotide linkage, a 2′-O-methyl ribonucleotide, a 2′-deoxy-2′-fluoro ribonucleotide, a 2′-deoxy ribonucleotide, a universal base nucleotide, a 5-C methyl nucleotide, an inverted deoxybasic residue incorporation, or a locked nucleic acid.

As used herein, the term “miR142” or “miR142 nucleic acid sequence” includes all forms of miR142 including the pri-, pre-, and mature forms of miR142, as well as variants, homologues, modifications, and derivatives thereof (e.g., within at least 50%, 80%, 90%, 95%, 96%, 97%, 98%, 99% or 100% activity compared to the native miR142). In embodiments, the variants or homologues or derivatives have at least 50%, 80%, 85%, 90%, 91%, 92%, 93%, 94%, 95%, 96%, 97%, 98%, 99% or 100% sequence identity across the whole sequence or a portion of the sequence (e.g. a 10, 15, 20, 25, 30, 40, 45, 50, 55, 60, 65, 70, 75, or 80 continuous nucleotides portion) compared to a naturally occurring form. In embodiments, the miR142 is the miRNA as identified by NCBI Reference Sequence: NR_029683.1.

As used herein, the term “miR142-mimic” or “miR142-mimic nucleic acid sequence” refers to an oligonucleotide that is structurally substantially similar to miR142 and is capable of effecting a biological function similar to miR142. In embodiments, the miR142-mimic has at least 50%, 80%, 90%, 95%, 96%, 97%, 98%, 99% or 100% activity compared to the native miR142. In embodiments, the miR142-mimic has at least 50%, 80%, 85%, 90%, 91%, 92%, 93%, 94%, 95%, 96%, 97%, 98%, 99% or 100% sequence identity across the whole sequence or a portion of the sequence (e.g. a 10, 15, 20, 25, 30, 40, 45, 50, 55, 60, 65, 70, 75, or 80 continuous nucleotides portion) compared to native miR142.

The term “miR-142 level” or “level of miR-142” refers to the expression levels of miR-142 in a biological sample. In embodiments, determining the level of expression of miR-142 includes calculating the mean of Log 2 of the expression of miR-142 in a biological sample. In embodiments, miR-142 expression is determined by Nanostring counts. In embodiments, miR-142 expression is determined by number of transcripts detected in the sample. One skilled in the art could use other methods for quantifying miR-142 expression, such as RNAseq or quantitative PCR.

The term “reduced level of miR142” or “reduced expression level of “miR142” refers to an expression level of miR-142 that is lower than the expression level of miR-142 in a control. The control may be any suitable control, examples of which are described herein. miR-142 expression levels may be detected by any known methodology, including but not limited to rtPCR, RNA sequencing, nanopore sequencing, microarray, hybridization-based sequencing, hybridization-based detection and quantification (e.g., NanoString).

The term “phosphorothioated miRNA” and “phosphorothioated miRNA-mimic” refers to a nucleic acid sequence in which one or more of the internucleotide linkages constitute a phosphorothioate linkage. In embodiments, a phosphorothioated miRNA is 5 to 30 bases long, single-stranded, partly, or completely phosphorothioated. In embodiments, phosphorothioated miRNA is 10 to 30 bases long, single-stranded, partly or completely phosphorothioated. In embodiments, phosphorothioated miRNA is 15 to 25 bases long, single-stranded, partly or completely phosphorothioated. In embodiments, the phosphorothioated miRNA is a miRNA in which 1, 2, 3, 4, 5, 6, 7, 8, 9, 10, 11, 12, 13, 14, 15, 16, 17, 18, 19, 20, 21, 22, 23, 24, 25, 26, 27, or 28, internucleotide linkages constitute a phosphorothioate linkage. In embodiments, the phosphorothioated miRNA is a miRNA in which 1 to 10 internucleotide linkages constitute a phosphorothioate linkage. In embodiments, the phosphorothioated miRNA is a miRNA in which 1 to 5 internucleotide linkages constitute a phosphorothioate linkage. In embodiments, the phosphorothioated miRNA is a miRNA in which 1 or 2 internucleotide linkages constitute a phosphorothioate linkage. In embodiments, the phosphorothioated miRNA is a miRNA in which all the internucleotide linkages constitute a phosphorothioate linkage. In embodiments, the 3′terminal nucleic acid in the phosphorothioated miRNA is a phosphorothioated nucleotide, which is encompassed by the term “phosphorothioate linkage.”

The term “Toll-like receptor 9” or “TLR9” refers to any of the recombinant or naturally-occurring forms of the TLR9 protein or variants or homologs thereof that maintain TLR9 protein activity (e.g. within at least 50%, 80%, 90%, 95%, 96%, 97%, 98%, 99% or 100% activity compared to the TLR9 receptor). In embodiments, the variants or homologs have at least 90%, 95%, 96%, 97%, 98%, 99% or 100% amino acid sequence identity across the whole sequence or a portion of the sequence (e.g. a 5, 10, 15, or 20 continuous amino acid portion) compared to a naturally occurring TLR9 receptor polypeptide. In embodiments, the TLR9 receptor protein is substantially identical to or identical to the protein identified by UniProtKB reference number Q9NR96, or a variant or homolog having substantial identity thereto.

A “toll-like receptor 9-binding nucleic acid sequence” refers to a nucleic acid capable of binding to toll like receptor 9. Exemplary nucleic acids include CpG oligodeoxynucleotides.

The term “CpG oligodeoxynucleotide” or “CpG ODN” refers to a 5′ C nucleotide connected to a 3′ G nucleotide through a phosphodiester internucleotide linkage or a phosphodiester derivative internucleotide linkage. In embodiments, a CpG ODN includes a phosphodiester internucleotide linkage. In embodiments, a CpG ODN includes a phosphodiester derivative internucleotide linkage.

The term “phosphorothioated oligodeoxynucleotide (ODN)” refers to a nucleic acid sequence, e.g., “CpG nucleic acid sequence” or “GpC nucleic acid sequence” in which one, some, or all the internucleotide linkages constitute a phosphorothioate linkage. In embodiments, phosphorothioated oligodeoxynucleotide (ODN) is 5 to 30 bases long, single-stranded, partly or completely phosphorothioated. The partly phosphorothioated ODN is an ODN in which 1, 2, 3, 4, 5, 6, 7, 8, 9, 10, 11, 12, 13, 14, 15, 16, 17, 18, 19, 20, 21, 22, 23, 24, 25, 26, 27, or 28, internucleotide linkages constitute a phosphorothioate linkage.

The term “Class A CpG ODN” or “A-class CpG ODN” or “D-type CpG ODN” or “Class A CpG DNA sequence” refers to a CpG motif including oligodeoxynucleotide including one or more of poly-G sequence at the 5′, 3′, or both ends; an internal palindrome sequence including CpG motif; or one or more phosphodiester derivatives linking deoxynucleotides. In embodiments, a Class A CpG ODN includes poly-G sequence at the 5′, 3′, or both ends; an internal palindrome sequence including CpG motif; and one or more phosphodiester derivatives linking deoxynucleotides. In embodiments, the phosphodiester derivative is phosphorothioate Examples of Class A CpG ODNs include ODN D19, ODN 1585, ODN 2216, and ODN 2336, the sequences of which are known in the art.

The term “Class B CpG ODN” or “B-class CpG ODN” or “K-type CpG ODN” or “Class B CpG DNA sequence” refers to a CpG motif including oligodeoxynucleotide including one or more of a 6mer motif including a CpG motif; phosphodiester derivatives linking all deoxynucleotides. In embodiments, a Class B CpG ODN includes one or more copies of a 6mer motif including a CpG motif and phosphodiester derivatives linking all deoxynucleotides. In embodiments, the phosphodiester derivative is phosphorothioate. In embodiments, a Class B CpG ODN includes one 6mer motif including a CpG motif. In embodiments, a Class B CpG ODN includes two copies of a 6mer motif including a CpG motif. In embodiments, a Class B CpG ODN includes three copies of a 6mer motif including a CpG motif. In embodiments, a Class B CpG ODN includes four copies of a 6mer motif including a CpG motif. Examples of Class B CpG ODNs include ODN 1668, ODN 1826, ODN 2006, ODN 2007, ODN BW006, and ODN D-SL01, the sequences of which are known in the art.

The term “Class C CpG ODN” or “C-class CpG ODN”” or “C-type CpG DNA sequence” refers to an oligodeoxynucleotide including a palindrome sequence including a CpG motif and phosphodiester derivatives (phosphorothioate) linking all deoxynucleotides. Examples of Class C CpG ODNs include ODN 2395, ODN M362, and ODN D-SL03, the sequences of which are known in the art.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides, contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

The term “phosphorothioated nucleotide” or “nucleotide with a phosphorothioate moiety” refers to a nucleotide having the structure:

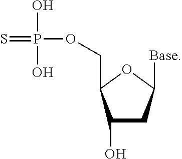

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

An “antisense nucleic acid” as referred to herein is a nucleic acid (e.g., DNA or RNA molecule) that is complementary to at least a portion of a specific target nucleic acid and is capable of reducing transcription of the target nucleic acid (e.g. mRNA from DNA), reducing the translation of the target nucleic acid (e.g. mRNA), altering transcript splicing (e.g. single stranded morpholino oligo), or interfering with the endogenous activity of the target nucleic acid. Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid. In embodiments, the antisense nucleic acid hybridizes to the target nucleic acid in vitro. In embodiments, the antisense nucleic acid hybridizes to the target nucleic acid in a cell. In embodiments, the antisense nucleic acid hybridizes to the target nucleic acid in an organism. In embodiments, the antisense nucleic acid hybridizes to the target nucleic acid under physiological conditions. Antisense nucleic acids may comprise naturally occurring nucleotides or modified nucleotides such as, e.g., phosphorothioate, methylphosphonate, and anomeric sugar-phosphate, backbone-modified nucleotides.

In the cell, the antisense nucleic acids hybridize to the corresponding RNA forming a double-stranded molecule. The antisense nucleic acids interfere with the endogenous behavior of the RNA and inhibit its function relative to the absence of the antisense nucleic acid. Furthermore, the double-stranded molecule may be degraded via the RNAi pathway. The use of antisense methods to inhibit the in vitro translation of genes is well known in the art (Marcus-Sakura, Anal. Biochem, 172:289, (1988)). Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogs), short hairpin RNAs (shRNA), micro RNAs (miRNA), saRNAs (small activating RNAs) and small nucleolar RNAs (snoRNA) or certain of their derivatives or pre-cursors.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region). In embodiments, the miR142 guide strand sequence has at least 85% sequence identity to the complement of at least 10 consecutive nucleotides of any one of SEQ ID NOS:1-3, 26, and 27. In embodiments, the miR142 guide strand sequence has at least 90% sequence identity to the complement of at least 10 consecutive nucleotides of any one of SEQ ID NOS:1-3, 26, and 27. In embodiments, the miR142 guide strand sequence has at least 95% sequence identity to the complement of at least 10 consecutive nucleotides of any one of SEQ ID NOS:1-3, 26, and 27. In embodiments, the miR142 guide strand sequence has at least 85% sequence identity to the complement of at least 15 consecutive nucleotides of any one of SEQ ID NOS:1-3, 26, and 27. In embodiments, the miR142 guide strand sequence has at least 90% sequence identity to the complement of at least 15 consecutive nucleotides of any one of SEQ ID NOS:1-3, 26, and 27. In embodiments, the miR142 guide strand sequence has at least 95% sequence identity to the complement of at least 15 consecutive nucleotides of any one of SEQ ID NOS:1-3, 26, and 27. In embodiments, miR142 guide strand sequence has at least 85% sequence identity to the complement of at least 18 consecutive nucleotides of any one of SEQ ID NOS:1-3, 26, and 27. In embodiments, the miR142 guide strand sequence has at least 90% sequence identity to the complement of at least 18 consecutive nucleotides of any one of SEQ ID NOS:1-3, 26, and 27. In embodiments, the miR142 guide strand sequence has at least 95% sequence identity to the complement of at least 18 consecutive nucleotides of any one of SEQ ID NOS:1-3, 26, and 27. In embodiments, miR142 guide strand sequence has at least 85% sequence identity to the complement of all nucleotides of any one of SEQ ID NOS:1-3, 26, and 27. In embodiments, the miR142 guide strand sequence has at least 90% sequence identity to the complement of all nucleotides of any one of SEQ ID NOS:1-3, 26, and 27. In embodiments, the miR142 guide strand sequence has at least 95% sequence identity to the complement of all nucleotides of any one of SEQ ID NOS:1-3, 26, and 27. In embodiments, the miR142 guide strand of any one of SEQ ID NOS:4, 5, and 28 comprises one or more 2′-O-Methyl-modified nucleic acids, one or more 2′-Fluoro-modified nucleic acids, or a combination thereof. In embodiments, the miR142 guide strand of any one of SEQ ID NOS:4, 5, and 28 comprises one or more 2′-O-Methyl-modified nucleic acids. In embodiments, the miR142 guide strand of any one of SEQ ID NOS:4, 5, and 28 comprises one or more 2′-Fluoro-modified nucleic acids. In embodiments, the miR142 guide strand of any one of SEQ ID NOS:4, 5, and 28 comprises one or more phosphorothioate bonds. In embodiments, the miR142 guide strand of any one of SEQ ID NOS:4, 5, and 28 comprises one or more 2′-O-Methyl-modified nucleic acids, one or more 2′-Fluoro-modified nucleic acids, one or more phosphorothioate bonds, or a combination thereof. In embodiments, the miR142 guide strand of any one of SEQ ID NOS:4, 5, and 28 comprises one or more 2′-O-Methyl-modified nucleic acids and one or more phosphorothioate bonds. In embodiments, the miR146a guide strand of any one of SEQ ID NOS:4, 5, and 28 comprises one or more 2′-Fluoro-modified nucleic acids and one or more phosphorothioate bonds.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “heterologous” when used with reference to portions of a nucleic acid indicates that the nucleic acid including two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein including two or more subsequences that are not found in the same relationship to each other in nature (e.g., a fusion protein).

The phrase “specifically (or selectively) binds” to an antibody or “specifically (or selectively) immunoreactive with,” when referring to a protein or peptide (e.g., binding of CpG to TLR9), refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “labeled nucleic acid or oligonucleotide” is one that is bound, either covalently, through a linker or a chemical bond, or noncovalently, through ionic, van der Waals, electrostatic, or hydrogen bonds to a label such that the presence of the nucleic acid may be detected by detecting the presence of the detectable label bound to the nucleic acid. Alternatively, a method using high affinity interactions may achieve the same results where one of a pair of binding partners binds to the other, e.g., biotin, streptavidin. In embodiments, the phosphorothioate nucleic acid or phosphorothioate polymer backbone includes a detectable label, as disclosed herein and known in the art.

The terms “isolate” or “isolated,” when applied to a nucleic acid, virus, or protein, denotes that the nucleic acid, virus, or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (e.g., www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

A “therapeutic agent” as used herein refers to an agent (e.g., nucleic acid, compound, or pharmaceutical composition described herein) that when administered to a subject will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms or the intended therapeutic effect, e.g., treatment or amelioration of an injury, disease, pathology or condition, or their symptoms including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being.

“Contacting” is used in accordance with its plain ordinary meaning and refers to the process of allowing at least two distinct species (e.g. chemical compounds including biomolecules or cells) to become sufficiently proximal to react, interact, or physically touch. It should be appreciated; however, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a nucleic acid as described herein and a cell, protein, or enzyme.

The term “control,” “suitable control,” or “control experiment” is used in accordance with its plain ordinary meaning and refers to an experiment in which the subjects or reagents of the experiment are treated as in a parallel experiment except for omission of a procedure, reagent, or variable of the experiment. In embodiments, the control is used as a standard of comparison in evaluating experimental effects. In embodiments, a control is the measurement of the activity of a protein or nucleic acid in the absence of a compound as described herein (including embodiments and examples). For example, a test sample can be taken from a patient suspected of having myeloid leukemia and compared to samples from a known cancer patient, or a known normal (non-disease) individual. A control can also represent an average value gathered from a population of similar individuals, e.g., cancer patients or healthy individuals with a similar medical background, same age, weight, etc. A control value can also be obtained from the same individual, e.g., from an earlier-obtained sample, prior to disease, or prior to treatment. One of skill will recognize that controls can be designed for assessment of any number of parameters. In embodiments, a control is a negative control. In embodiments, such as some embodiments relating to detecting the level of expression of a gene/protein or a subset of genes/proteins (e.g., the level of miR-142), a control comprises the average amount of expression (e.g., protein or mRNA) in a population of subjects (e.g., with cancer) or in a healthy or general population. In embodiments, the control comprises an average amount (e.g. amount of expression) in a population in which the number of subjects (n) is 5 or more, 6 or more, 7 or more, 8 or more, 9 or more, 10 or more, 25 of more, 50 or more, 100 or more, 1000 or more, 5000 or more, or 10000 or more. In embodiments, the control is a standard control. In embodiments, the control is a population of cancer subjects. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

A “detectable agent” or “detectable moiety” is a compound or composition detectable by appropriate means such as spectroscopic, photochemical, biochemical, immunochemical, chemical, magnetic resonance imaging, or other physical means. A detectable moiety is a monovalent detectable agent or a detectable agent bound (e.g. covalently and directly or via a linking group) with another compound, e.g., a nucleic acid. Exemplary detectable agents/moieties for use in the present disclosure include an antibody ligand, a peptide, a nucleic acid, radioisotopes, paramagnetic metal ions, fluorophore (e.g. fluorescent dyes), electron-dense reagents, enzymes (e.g., as commonly used in an ELISA), biotin, a biotin-avidin complex, a biotin-streptavidin complex, digoxigenin, magnetic beads (e.g., DYNABEADS® by ThermoFisher, encompassing functionalized magnetic beads such as DYNABEADS® M-270 amine by ThermoFisher), paramagnetic molecules, paramagnetic nanoparticles, ultrasmall superparamagnetic iron oxide nanoparticles, ultrasmall superparamagnetic iron oxide nanoparticle aggregates, superparamagnetic iron oxide nanoparticles, superparamagnetic iron oxide nanoparticle aggregates, monocrystalline iron oxide nanoparticles, monocrystalline iron oxide, nanoparticle contrast agents, liposomes or other delivery vehicles containing Gadolinium chelate molecules, gadolinium, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. In embodiments, the compounds described herein comprise a detectable moiety.

“Fluorophore” refers to compounds that absorb light energy of a specific wavelength and re-emit the light at a lower wavelength. Exemplary fluorophores that may be used herein include xanthenes (e.g., fluorescein, rhodamine, Oregon green, eosin, Texas red); cyanines (e.g., cyanine, indocarbocyanine, oxacarbocyanine, thiacarbocyanine, merocyanine); squaraines (e.g., Seta, Square dyes); squaraine rotaxane (e.g., SeTau® dyes); naphthalenes (e.g., dansyl, prodan); coumarins; oxadiazoles (e.g., pyridyloxazole, nitrobenzoxadiazole, benzooxadiazole); anthracenes (e.g., anthraquinones, DRAQ5®, DRAQ7®, CyTRAK® orange); pyrenes (e.g., cascade blue); oxazines (e.g., Nile red, Nile blue, cresyl violet, oxazine 170); acridines (e.g., proflavin, acridine orange, acridine yellow); arylmethines (e.g., auramine, crystal violet, malachite green); tetrapyrroles (e.g., porphin, phthalocyanine, bilirubin), and the like. In embodiments, “fluorophore” is a fluorophore bound to avidin (e.g., Alexa Fluor® Avidin by ThermoFisher; or Rhodamine Avidin, Fluorescein Avidin, Texas Red® Aavidin all by Vector Laboratories). In embodiments, “fluorophore” is a fluorophore bound to streptavidin (e.g., Alexa Fluor® Streptavidin by ThermoFisher; or DyLight Streptavidin, Cy3 Streptavidin, Fluorescein Streptavidin, Texas Red® Streptavidin all by Vector Laboratories).

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched non-cyclic carbon chain (or carbon), or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable non-cyclic straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃.

The term “heteroalkylene,” by itself or as part of another substituent, means, unless otherwise stated, a divalent radical derived from heteroalkyl, as exemplified, but not limited by, —CH₂—CH₂—S—CH₂—CH₂—, —O—CH₂—CH₂—NH—CH₂—, —O—(CH₂)₃—O—PO₃—, —O—(CH₂)—O—PO₃—, —O—(CH₂)₂—O—PO₃—, —O—(CH₂)₄—O—PO₃—, and the like. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic non-aromatic versions of “alkyl” and “heteroalkyl,” respectively, wherein the carbons making up the ring or rings do not necessarily need to be bonded to a hydrogen due to all carbon valencies participating in bonds with non-hydrogen atoms. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently (e.g., biphenyl). A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. Non-limiting examples of heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

The term “oxo” means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, =NR′, =N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)=NR″″, —NR—C(NR′R″)=NR′″, —S(O)R′, —S(O)₂R′, —S(O)₂NR′R″, —NRSO₂R′, —NR′NR″R′″, —ONR′R″, NR′C═(O)NR″NR′″R″″, —CN, —NO₂, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)=NR″″, —NR—C(NR′R″)=NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In embodiments, the ring-forming substituents are attached to adjacent members of the base structure. For example, two ring-forming substituents attached to adjacent members of a cyclic base structure create a fused ring structure. In embodiments, the ring-forming substituents are attached to a single member of the base structure. For example, two ring-forming substituents attached to a single member of a cyclic base structure create a spirocyclic structure. In embodiments, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties:
(A) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, heteroaryl, substituted with at least one substituent selected from:
(i) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, and
(ii) alkyl, heteroalkyl, cycloalkyl, heterocycloalkyl, aryl, heteroaryl, substituted with at least one substituent selected from: (a) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, and (b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In embodiments, each substituted group described in the compounds herein is substituted with at least one substituent group. More specifically, in embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In embodiments, at least one or all of these groups are substituted with at least one size-limited substituent group. In embodiments, at least one or all of these groups are substituted with at least one lower substituent group.

In embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In embodiments, each substituted or unsubstituted alkyl is a substituted or unsubstituted C₁-C₈alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In embodiments, the compound is a chemical species set forth in the Examples section below.

As defined herein, the term “activation”, “activate”, “activating”, “activator” and the like in reference to a protein-inhibitor interaction means positively affecting (e.g. increasing) the activity or function of the protein relative to the activity or function of the protein in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In embodiments, expression or activity is 1.5-fold, 2-fold, 3-fold, 4-fold, 5-fold, 10-fold, or higher than the expression or activity in the absence of the agonist.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; or rodent.

### Compounds

The disclosure provides a hybridized nucleic acid sequence, where a microRNA-142 passenger strand sequence is hybridized to a microRNA-142 guide strand sequence. In embodiments, the miR142 passenger and guide strand sequences are miR142-mimic passenger and guide strand sequences. In embodiments, the hybridized nucleic acid sequences are covalently bonded to a Toll-like receptor 9-binding nucleic acid sequence. In embodiments, the microRNA-142 passenger strand sequence is covalently bonded to the Toll-like receptor 9-binding nucleic acid sequence; and the microRNA-142 guide strand sequence is hybridized to the microRNA passenger strand sequence.

The disclosure provides a hybridized nucleic acid sequence, where a miR142 passenger strand sequence is hybridized to a miR142 guide strand sequence. In embodiments, the miR142 passenger strand sequence comprises SEQ ID NO: 1, SEQ ID NO:2, SEQ ID NO:3, SEQ ID NO:26, or SEQ ID NO:27, and the miR142 guide strand sequence comprises SEQ ID NO:4, SEQ ID NO:5, or SEQ ID NO:28. In embodiments, the miR142 passenger strand sequence comprises SEQ ID NO: 1, SEQ ID NO:2, or SEQ ID NO:3, and the miR142 guide strand sequence comprises SEQ ID NO:4, or SEQ ID NO:5. In embodiments, the miR142 passenger strand sequence comprises SEQ ID NO:1 or SEQ ID NO:2, and the miR142 guide strand sequence comprises SEQ ID NO:4. In embodiments, the miR142 passenger strand sequence comprises SEQ ID NO: 1, and the miR142 guide strand sequence comprises SEQ ID NO:4. In embodiments, the miR142 passenger strand sequence comprises SEQ ID NO:2, and the miR142 guide strand sequence comprises SEQ ID NO:4. In embodiments, the miR142 passenger strand sequence comprises SEQ ID NO:3, and the miR142 guide strand sequence comprises SEQ ID NO:5. In embodiments, the miR142 passenger strand sequence comprises SEQ ID NO:26, and the miR142 guide strand sequence comprises SEQ ID NO:4. In embodiments, the miR142 passenger strand sequence comprises SEQ ID NO:27, and the miR142 guide strand sequence comprises SEQ ID NO:28. In embodiments, the hybridized nucleic acid sequences are covalently bonded to a Toll-like receptor 9-binding nucleic acid sequence. In embodiments, the miR142 passenger strand sequence is covalently bonded to the Toll-like receptor 9-binding nucleic acid sequence; and the miR142 guide strand sequence is hybridized to the miR142 passenger strand sequence.

The disclosure provides compounds of Formula (A):

R¹-L¹-R² (A);

where R¹ is a Toll-like receptor 9-binding nucleic acid sequence; L¹ is a linking group; and R² is a nucleic acid sequence comprising a miR142 passenger sequence. In embodiments, the 3′ end of the miR142 passenger strand sequence is bonded to L¹. In embodiments, the 5′ end of the miR142 passenger strand sequence is bonded to L¹. In embodiments, the Toll-like receptor 9-binding nucleic acid sequence comprises a phosphorothioate linkage. In embodiments, the miR-142 passenger strand sequence comprises SEQ ID NO: 1, SEQ ID NO:2, SEQ ID NO:3, SEQ ID NO:26, or SEQ ID NO:27. In embodiments, the miR-142 passenger strand sequence comprises SEQ ID NO: 1. In embodiments, the miR-142 passenger strand sequence comprises SEQ ID NO:2. In embodiments, the miR-142 passenger strand sequence comprises SEQ ID NO:3. In embodiments, the miR-142 passenger strand sequence comprises SEQ ID NO:26. In embodiments, the miR-142 passenger strand sequence comprises SEQ ID NO:27.

The disclosure provides compounds of Formula (I):

R¹-L¹-R² (I);

where R¹ is a Toll-like receptor 9-binding nucleic acid sequence; L¹ is a linking group; and R² is a hybridized nucleic acid sequence comprising a miR142 passenger sequence hybridized to a miR142 guide strand sequence. In embodiments, the 3′ end of the miR142 passenger strand sequence is bonded to L¹. In embodiments, the 5′ end of the miR142 passenger strand sequence is bonded to L¹. In embodiments, the Toll-like receptor 9-binding nucleic acid sequence comprises a phosphorothioate linkage.

In embodiments of the compound of Formula (I), the miR142 passenger strand sequence comprises SEQ ID NO: 1; and the miR142 guide strand sequence comprises SEQ ID NO:4. In embodiments, SEQ ID NO:1 comprises a 2′-O-methyl nucleotide, a 2′-fluoro-nucleotide, a phosphorothioate linkage, or a combination thereof. In embodiments, SEQ ID NO:1 comprises a 2′-O-methyl nucleotide. In embodiments, SEQ ID NO:1 comprises a 2′-fluoro-nucleotide. In embodiments, SEQ ID NO:1 comprises a phosphorothioate linkage. In embodiments, SEQ ID NO:1 comprises a 2′-O-methyl nucleotide and a phosphorothioate linkage. In embodiments, SEQ ID NO:1 comprises a 2′-fluoro-nucleotide and a phosphorothioate linkage. In embodiments, SEQ ID NO:4 comprises a 2′-O-methyl nucleotide, a 2′-fluoro-nucleotide, a phosphorothioate linkage, or a combination thereof. In embodiments, SEQ ID NO:4 comprises a 2′-O-methyl nucleotide. In embodiments, SEQ ID NO:4 comprises a 2′-fluoro-nucleotide. In embodiments, SEQ ID NO:4 comprises a phosphorothioate linkage. In embodiments, SEQ ID NO:4 comprises a 2′-O-methyl nucleotide and a phosphorothioate linkage. In embodiments, SEQ ID NO:4 comprises a 2′-fluoro-nucleotide and a phosphorothioate linkage. In embodiments, the microRNA-142 passenger strand sequence is covalently bonded via the linking group to the Toll-like receptor 9-binding nucleic acid sequence; and the microRNA-142 guide strand sequence is hybridized to the microRNA passenger strand sequence. In embodiments, the 3′ end of the microRNA-142 passenger strand sequence is covalently bonded via the linking group to the 3′ end of the Toll-like receptor 9-binding nucleic acid sequence; and the microRNA-142 guide strand sequence is hybridized to the microRNA passenger strand sequence. In embodiments, the 5′ end of the microRNA-142 passenger strand sequence is covalently bonded via the linking group to the 3′ end of the Toll-like receptor 9-binding nucleic acid sequence; and the microRNA-142 guide strand sequence is hybridized to the microRNA passenger strand sequence.

In embodiments of the compound of Formula (I), the miR142 passenger strand sequence comprises SEQ ID NO:2; and the miR142 guide strand sequence comprises SEQ ID NO:4. In embodiments, SEQ ID NO:2 comprises a 2′-O-methyl nucleotide, a 2′-fluoro-nucleotide, a phosphorothioate linkage, or a combination thereof. In embodiments, SEQ ID NO:2 comprises a 2′-O-methyl nucleotide. In embodiments, SEQ ID NO:2 comprises a 2′-fluoro-nucleotide. In embodiments, SEQ ID NO:2 comprises a phosphorothioate linkage. In embodiments, SEQ ID NO:2 comprises a 2′-O-methyl nucleotide and a phosphorothioate linkage. In embodiments, SEQ ID NO:2 comprises a 2′-fluoro-nucleotide and a phosphorothioate linkage. In embodiments, SEQ ID NO:4 comprises a 2′-O-methyl nucleotide, a 2′-fluoro-nucleotide, a phosphorothioate linkage, or a combination thereof. In embodiments, SEQ ID NO:4 comprises a 2′-O-methyl nucleotide. In embodiments, SEQ ID NO:4 comprises a 2′-fluoro-nucleotide. In embodiments, SEQ ID NO:4 comprises a phosphorothioate linkage. In embodiments, SEQ ID NO:4 comprises a 2′-O-methyl nucleotide and a phosphorothioate linkage. In embodiments, SEQ ID NO:4 comprises a 2′-fluoro-nucleotide and a phosphorothioate linkage. In embodiments, the microRNA-142 passenger strand sequence is covalently bonded via the linking group to the Toll-like receptor 9-binding nucleic acid sequence; and the microRNA-142 guide strand sequence is hybridized to the microRNA passenger strand sequence. In embodiments, the 3′ end of the microRNA-142 passenger strand sequence is covalently bonded via the linking group to the 3′ end of the Toll-like receptor 9-binding nucleic acid sequence; and the microRNA-142 guide strand sequence is hybridized to the microRNA passenger strand sequence. In embodiments, the 5′ end of the microRNA-142 passenger strand sequence is covalently bonded via the linking group to the 3′ end of the Toll-like receptor 9-binding nucleic acid sequence; and the microRNA-142 guide strand sequence is hybridized to the microRNA passenger strand sequence.

In embodiments of the compound of Formula (I), the miR142 passenger strand sequence comprises SEQ ID NO:3 and the miR142 guide strand sequence comprises SEQ ID NO:5. In embodiments, the microRNA-142 passenger strand sequence is covalently bonded via the linking group to the Toll-like receptor 9-binding nucleic acid sequence; and the microRNA-142 guide strand sequence is hybridized to the microRNA passenger strand sequence. In embodiments, the 3′ end of the microRNA-142 passenger strand sequence is covalently bonded via the linking group to the 3′ end of the Toll-like receptor 9-binding nucleic acid sequence; and the microRNA-142 guide strand sequence is hybridized to the microRNA passenger strand sequence. In embodiments, the 5′ end of the microRNA-142 passenger strand sequence is covalently bonded via the linking group to the 3′ end of the Toll-like receptor 9-binding nucleic acid sequence; and the microRNA-142 guide strand sequence is hybridized to the microRNA passenger strand sequence.

In embodiments of the compound of Formula (I), the miR142 passenger strand sequence comprises SEQ ID NO:26 and the miR142 guide strand sequence comprises SEQ ID NO:4. In embodiments, the microRNA-142 passenger strand sequence is covalently bonded via the linking group to the Toll-like receptor 9-binding nucleic acid sequence; and the microRNA-142 guide strand sequence is hybridized to the microRNA passenger strand sequence. In embodiments, the 3′ end of the microRNA-142 passenger strand sequence is covalently bonded via the linking group to the 3′ end of the Toll-like receptor 9-binding nucleic acid sequence; and the microRNA-142 guide strand sequence is hybridized to the microRNA passenger strand sequence. In embodiments, the 5′ end of the microRNA-142 passenger strand sequence is covalently bonded via the linking group to the 3′ end of the Toll-like receptor 9-binding nucleic acid sequence; and the microRNA-142 guide strand sequence is hybridized to the microRNA passenger strand sequence.

In embodiments of the compound of Formula (I), the miR142 passenger strand sequence comprises SEQ ID NO:27 and the miR142 guide strand sequence comprises SEQ ID NO:28. In embodiments, the microRNA-142 passenger strand sequence is covalently bonded via the linking group to the Toll-like receptor 9-binding nucleic acid sequence; and the microRNA-142 guide strand sequence is hybridized to the microRNA passenger strand sequence. In embodiments, the 3′ end of the microRNA-142 passenger strand sequence is covalently bonded via the linking group to the 3′ end of the Toll-like receptor 9-binding nucleic acid sequence; and the microRNA-142 guide strand sequence is hybridized to the microRNA passenger strand sequence. In embodiments, the 5′ end of the microRNA-142 passenger strand sequence is covalently bonded via the linking group to the 3′ end of the Toll-like receptor 9-binding nucleic acid sequence; and the microRNA-142 guide strand sequence is hybridized to the microRNA passenger strand sequence.

In embodiments of all the compounds described herein, the microRNA-142 passenger strand sequence further comprises from 1 to 10 nucleotides on the 3′ end, the ′5 end, or both the 3′ and 5′ end; wherein 1 to 10 nucleotides are modified with 2′-O-Methyl, 2′-Fluoro, a phosphorothioate linkage, or a combination thereof. In embodiments, the microRNA-142 passenger strand sequence further comprises from 1 to 8 nucleotides on the 3′ end, the ′5 end, or both the 3′ and 5′ end; wherein 1 to 8 nucleotides are modified with 2′-O-Methyl, 2′-Fluoro, a phosphorothioate linkage, or a combination thereof. In embodiments, the microRNA-142 passenger strand sequence further comprises from 1 to 6 nucleotides on the 3′ end, the ′5 end, or both the 3′ and 5′ end; wherein 1 to 6 nucleotides are modified with 2′-O-Methyl, 2′-Fluoro, a phosphorothioate linkage, or a combination thereof. In embodiments, the microRNA-142 passenger strand sequence further comprises from 1 to 5 nucleotides on the 3′ end, the ′5 end, or both the 3′ and 5′ end; wherein 1 to 5 nucleotides are modified with 2′-O-Methyl, 2′-Fluoro, a phosphorothioate linkage, or a combination thereof. In embodiments, the microRNA-142 passenger strand sequence further comprises from 1 to 4 nucleotides on the 3′ end, the ′5 end, or both the 3′ and 5′ end; wherein 1 to 4 nucleotides are modified with 2′-O-Methyl, 2′-Fluoro, a phosphorothioate linkage, or a combination thereof. In embodiments, the microRNA-142 passenger strand sequence further comprises from 1 to 3 nucleotides on the 3′ end, the ′5 end, or both the 3′ and 5′ end; wherein 1 to 3 nucleotides are modified with 2′-O-Methyl, 2′-Fluoro, a phosphorothioate linkage, or a combination thereof. In embodiments, the microRNA-142 passenger strand sequence further comprises from 1 to 2 nucleotides on the 3′ end, the ′5 end, or both the 3′ and 5′ end; wherein 1 to 2 nucleotides are modified with 2′-O-Methyl, 2′-Fluoro, a phosphorothioate linkage, or a combination thereof.

In embodiments of all the compounds described herein, any one of SEQ ID NOS:1-5 and 26-28 further comprises from 1 to 10 nucleotides on the 3′ end, the ′5 end, or both the 3′ and 5′ end; wherein 1 to 10 nucleotides are modified with 2′-O-Methyl, 2′-Fluoro, a phosphorothioate linkage, or a combination thereof. In embodiments, any one of SEQ ID NOS: 1-5 and 26-28 further comprises from 1 to 8 nucleotides on the 3′ end, the ′5 end, or both the 3′ and 5′ end; wherein 1 to 8 nucleotides are modified with 2′-O-Methyl, 2′-Fluoro, a phosphorothioate linkage, or a combination thereof. In embodiments, any one of SEQ ID NOS: 1-5 and 26-28 further comprises from 1 to 6 nucleotides on the 3′ end, the ′5 end, or both the 3′ and 5′ end; wherein 1 to 6 nucleotides are modified with 2′-O-Methyl, 2′-Fluoro, a phosphorothioate linkage, or a combination thereof. In embodiments, any one of SEQ ID NOS: 1-5 and 26-28 further comprises from 1 to 5 nucleotides on the 3′ end, the ′5 end, or both the 3′ and 5′ end; wherein 1 to 5 nucleotides are modified with 2′-O-Methyl, 2′-Fluoro, a phosphorothioate linkage, or a combination thereof. In embodiments, any one of SEQ ID NOS: 1-5 and 26-28 further comprises from 1 to 4 nucleotides on the 3′ end, the ′5 end, or both the 3′ and 5′ end; wherein 1 to 4 nucleotides are modified with 2′-O-Methyl, 2′-Fluoro, a phosphorothioate linkage, or a combination thereof. In embodiments, any one of SEQ ID NOS: 1-5 and 26-28 further comprises from 1 to 3 nucleotides on the 3′ end, the ′5 end, or both the 3′ and 5′ end; wherein 1 to 3 nucleotides are modified with 2′-O-Methyl, 2′-Fluoro, a phosphorothioate linkage, or a combination thereof. In embodiments, any one of SEQ ID NOS: 1-5 and 26-28 further comprises 1 or 2 nucleotides on the 3′ end, the ′5 end, or both the 3′ and 5′ end; wherein 1 or 2 nucleotides are modified with 2′-O-Methyl, 2′-Fluoro, a phosphorothioate linkage, or a combination thereof. In embodiments, 1 or more of the additional nucleotides on the 3′ end of any one of SEQ ID NOS:1-5 and 26-28 are modified with 2′-O-Methyl, 2′-Fluoro, a phosphorothioate linkage, or a combination thereof. In embodiments, 1 or more of the additional nucleotides on the 5′ end of any one of SEQ ID NOS:1-5 and 26-28 are modified with 2′-O-Methyl, 2′-Fluoro, a phosphorothioate linkage, or a combination thereof. In embodiments, 1 or more of the additional nucleotides on both the 3′ end and the 5′ end of any one of SEQ ID NOS: 1-5 and 26-28 are modified with 2′-O-Methyl, 2′-Fluoro, a phosphorothioate linkage, or a combination thereof.

R¹ comprises a Toll-like receptor 9-binding nucleic acid sequence. In embodiments, the Toll-like receptor 9-binding nucleic acid sequence comprises a CpG oligodeoxynucleotide (ODN). In embodiments, the CpG ODN is a CpG-A ODN, a CpG-B ODN, a CpG-C ODN, or a combination of two or more thereof. In embodiments, the CpG ODN is a CpG-A ODN. In embodiments, the CpG ODN is a CpG-B ODN. In embodiments, the CpG ODN is a CpG-C ODN. In embodiments, the CpG ODN is CpG ODN D19, CpG ODN 1585, CpG ODN 2216, CpG ODN 2336, CpG ODN 1668, CpG ODN 1826, CpG ODN 2006, CpG ODN 2007, CpG ODN BW006, CpG ODN D-SL01, CpG ODN 2395, CpG ODN M362, CpG ODN D-SL03, or a combination of two or more thereof. In embodiments, the CpG ODN is CpG ODN D19. In embodiments, the CpG ODN is CpG ODN 1585. In embodiments, the CpG ODN is CpG ODN 2216. In embodiments, the CpG ODN is CpG ODN 2336. In embodiments, the CpG ODN is CpG ODN 1668. In embodiments, the CpG ODN is CpG ODN 1826. In embodiments, the CpG ODN is CpG ODN 2006. In embodiments, the CpG ODN is CpG ODN 2007. In embodiments, the CpG ODN is CpG ODN BW006. In embodiments, the CpG ODN is CpG ODN D-SL1. In embodiments, the CpG ODN is CpG ODN 2395. In embodiments, the CpG ODN is CpG ODN CpG ODN M362. In embodiments, the CpG ODN is CpG ODN D-SL03. In embodiments, the CpG oligodeoxynucleotide comprises one or more phosphorothioate linkages.

In embodiments, R¹ comprises a CpG-ODN nucleic acid sequence listed in Table 1.

TABLE 1

| | SEQ ID | SEQUENCE 5′-3′ |
|---|---|---|
| NAME | NO: | (* = phosphorothioate linkage) |
| CpG(A)-ODN | 6 | GGT GCA TCG ATG CAG GGG GG |
| CpG(A)-ODN | 7 | G*G*T GCA TCG ATG CAG G*G*G* G*G* |
| CpG(A)-ODN | 8 | G*G*T GCA TCG ATG CAG G*G*G* G*G |
| GpC(A)-ODN | 9 | G*G*T GCA TGC ATG CAG G*G*G*G*G |
| D19-PS | 10 | G*G*T*G*C*A*T*C*G*A*T*G*C*A*G*G*G*G*G*G |
| CpG(B)-ODN | 11 | T*C*C*A*T*G*A*C*G*T*T*C*C*T*G*A*T*G*C*T |
| ODN 1585 | 12 | G*G*GGTCAACGTTGAG*G*G*G*G*G |
| ODN 1585 | 13 | G*GGGTCAACGTTGAG*G*G*G*G*G |
| ODN 2216 | 14 | G*G*GGGACGATCGTCG*G*G*G*G*G |
| ODN 2216 | 15 | G*GGGGACGATCGTCG*G*G*G*G*G |
| ODN D19 | 16 | G*G*T GCA TCG ATG CAG GG*G* G*G |
| ODN D19 | 17 | G*GTGCATCGATGCAGG*G*G*G*G* |
| ODN 2336 | 18 | G*G*G*GACGACGTCGTGG*G*G*G*G*G |
| ODN 2336 | 19 | G*G*GGACGACGTCGTGG*G*G*G*G*G |
| ODN 1668 | 20 | T*C*C*A*T*G*A*C*G*T*T*C*C*T*G*A*T*G*C*T |
| ODN 1826 | 21 | T*C*C*A*T*G*A*C*G*T*T*C*C*T*G*A*C*G*T*T |
| ODN 2006 | 22 | T*C*G*T*C*G*T*T*T*T*G*T*C*G*T*T*T*T*G*T*C*G*T*T |
| ODN 2007 | 23 | T*C*G*T*C*G*T*T*G*T*C*G*T*T*T*T*G*T*C*G*T*T |
| ODN 2395 | 24 | T*C*G*T*C*G*T*T*T*T*C*G*G*C*G*C*G*C*G*C*C*G |
| ODN M362 | 25 | T*C*G*T*C*G*T*C*G*T*T*C*G*A*A*C*G*A*C*G*T*T*G*A*T |

L¹ is a bond, a nucleic acid sequence, a DNA sequence, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or a combination of two or more thereof. In embodiments, L¹ is a bond, a nucleic acid sequence, unsubstituted alkylene, unsubstituted heteroalkylene, or a combination of two or more thereof. In embodiments, L¹ is a covalent bond. In embodiments, L¹ is a nucleic acid sequence. In embodiments, L¹ is a nucleic acid sequence and a substituted or unsubstituted alkylene. In embodiments, L¹ is a nucleic acid sequence and an unsubstituted alkylene. In embodiments, L¹ is a nucleic acid sequence and a substituted or unsubstituted heteroalkylene. In embodiments, L¹ is a nucleic acid sequence and an unsubstituted heteroalkylene. In embodiments, L¹ is a substituted or unsubstituted heteroalkylene. In embodiments, L¹ is unsubstituted heteroalkylene. In embodiments, L¹ is a substituted or unsubstituted alkylene. In embodiments, L¹ is a substituted alkylene. In embodiments, L¹ is unsubstituted alkylene.

In embodiments, L¹ is substituted heteroalkylene. In embodiments, L¹ is substituted 6 to 60 membered heteroalkylene. In embodiments, L¹ is substituted 6 to 54 membered heteroalkylene. In embodiments, L¹ is substituted 12 to 48 membered heteroalkylene. In embodiments, L¹ is substituted 18 to 42 membered heteroalkylene. In embodiments, L¹ is substituted 24 to 36 membered heteroalkylene. In embodiments, L¹ is substituted 30 membered heteroalkylene. In embodiments, the heteroalkylene comprises an oxygen atom, a phosphorous atom, or a combination thereof. In embodiments, the substituents on the substituted heteroalkylene comprise oxo, —OH, —O⁻, or a combination of two or more thereof. In embodiments, L¹ is substituted 18 to 42 membered heteroalkylene; wherein the heteroalkylene comprises an oxygen atom, a phosphorous atom, or a combination thereof; and wherein the substituents are independently selected from the group consisting of oxo, —OH, and —O⁻.

In embodiments, L¹ is:

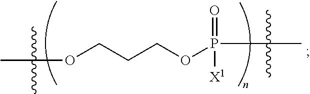

wherein X¹ is independently —OH or —O⁻, and n is an integer from 1 to 10. In embodiments, each X¹ is —OH. In embodiments, each X¹ is —O⁻. In embodiments, n is an integer from 2 to 10. In embodiments, n is an integer from 2 to 8. In embodiments, n is an integer from 3 to 7. In embodiments, n is an integer from 4 to 6. In embodiments, n is 1. In embodiments, n is 2. In embodiments, n is 3. In embodiments, n is 4. In embodiments, n is 5. In embodiments, n is 6. In embodiments, n is 7. In embodiments, n is 8. In embodiments, n is 9. In embodiments, n is 10.

In embodiments, L¹ is substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or a combination of two or more thereof. In embodiments, L¹ is a combination of two or three of substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene.

In embodiments, L¹ is substituted or unsubstituted heteroalkylene, substituted or unsubstituted heterocycloalkylene, substituted or unsubstituted heteroarylene, or a combination of two or more thereof. In embodiments, L¹ is a combination of substituted or unsubstituted heteroalkylene, substituted or unsubstituted heterocycloalkylene, and substituted or unsubstituted heteroarylene.

In embodiments, L¹ is substituted or unsubstituted heteroalkylene, wherein the substituted or unsubstituted heteroalkylene comprises —(CH₂CH₂O)—.

In embodiments, L¹ is a 5 or 6 membered substituted or unsubstituted heteroarylene. In embodiments, L¹ is a 5 or 6 membered substituted or unsubstituted heteroarylene comprising one or two nitrogen atoms. In embodiments, L¹ is a 5 or 6 membered substituted or unsubstituted heterocycloalkylene. In embodiments, L¹ is a 5 or 6 membered substituted or unsubstituted heterocycloalkylene comprising an oxygen atom, a nitrogen atom, or a combination thereof.

In embodiments, L¹ is a combination of two or more of (a) a 5 or 6 membered substituted or unsubstituted heteroarylene comprising one or two nitrogen atoms; (b) a 5 or 6 membered substituted or unsubstituted heterocycloalkylene comprising an oxygen atom, a nitrogen atom, or a combination thereof; and (c) substituted or unsubstituted heteroalkylene, wherein the substituted or unsubstituted heteroalkylene comprises —(CH₂CH₂O)—;

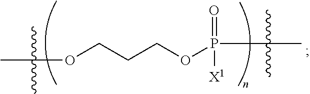

wherein X¹ is independently —OH or —O⁻, and n is an integer from 1 to 10; or a combination thereof.

In embodiments, L¹ is:

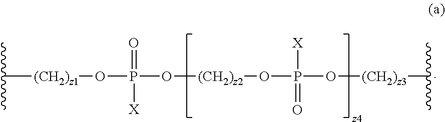

In embodiments, L¹ is:

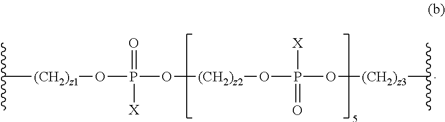

In embodiments, L¹ is:

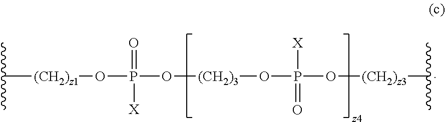

In embodiments, L¹ is:

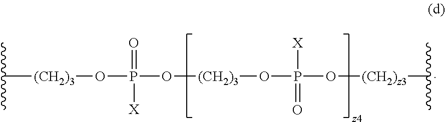

In embodiments, L¹ is:

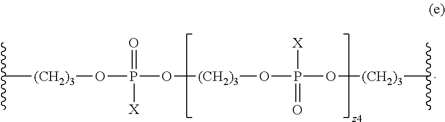

In embodiments, L¹ is:

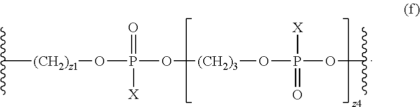

In embodiments, L¹ is:

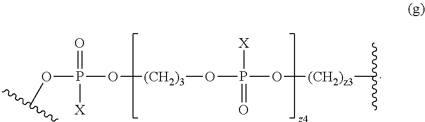

In embodiments, L¹ is:

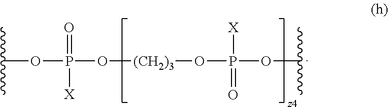

When L¹ is any of (a)-(h), z1, z2, z3 and z4 are independently integers from 0 to 20; and each X is independently —OH or —O⁻. In embodiments, z1 is an integer from 0 to 5. In embodiments, z1 is an integer from 2 to 4. In embodiments, z2 is an integer from 0 to 5. In embodiments, z2 is an integer from 2 to 4. In embodiments, z3 is an integer from 0 to 5. In embodiments, z1 is an integer from 2 to 4. In embodiments, z4 is an integer from 3 to 7. In embodiments, z4 is an integer from 4 to 6. In embodiments, each X is —OH. In embodiments, each X is —O⁻.

In embodiments, L¹ is:

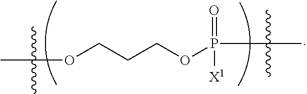

wherein X¹ is independently —OH or —O⁻.

In embodiments, L¹ is:

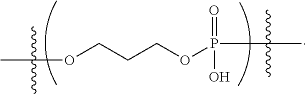

p is independently an integer from 1 to 10. In embodiments, p is independently an integer from 2 to 10. In embodiments, p is independently an integer from 2 to 8. In embodiments, p is independently an integer from 3 to 7. In embodiments, p is independently an integer from 4 to 6. In embodiments, p is 1. In embodiments, p is 2. In embodiments, p is 3. In embodiments, p is 4. In embodiments, p is 5. In embodiments, p is 6. In embodiments, p is 7. In embodiments, p is 8. In embodiments, p is 9. In embodiments, p is 10.

In embodiments, the nucleic acids described herein (e.g., Toll-like receptor 9-binding nucleic acid sequences, miR142 passenger strand sequences, miR142 guide strand sequences) comprises a terminal C3 spacer modification on the 5′-terminus, the 3′-terminus, or both the 5′ and 3′-terminus. In embodiments, the nucleic acids described herein comprise a terminal C3 spacer modification on the 5′-terminus. In embodiments, the nucleic acids described herein comprise a terminal C3 spacer modification on the 3′-terminus. In embodiments, the nucleic acids described herein comprise a terminal C3 spacer modification on both the 5′-terminus and the 3′-terminus. The term “terminal C3 unit” or “terminal C3 spacer modification” refers to a moiety of the following structure:

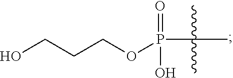

wherein X¹ is —OH or O⁻.

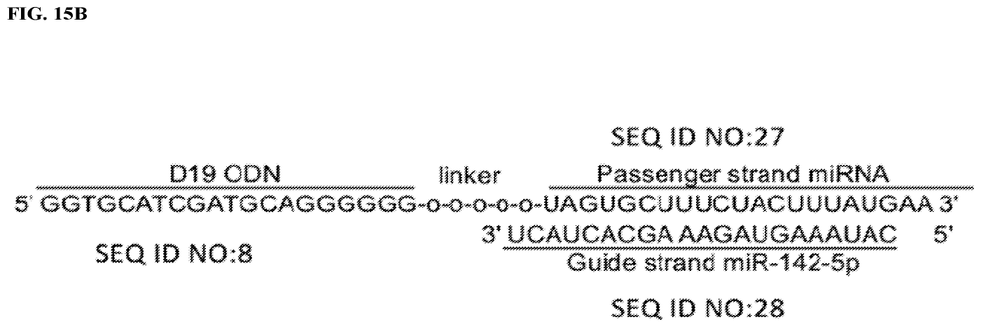

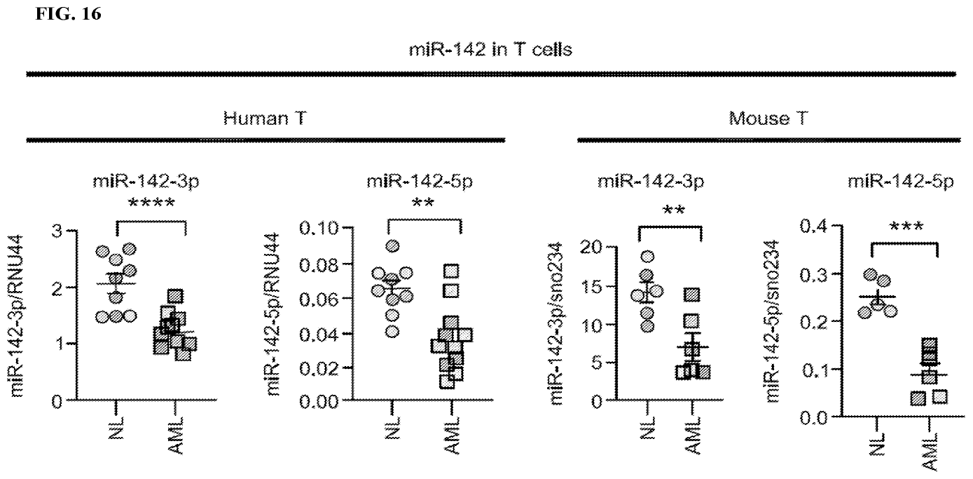

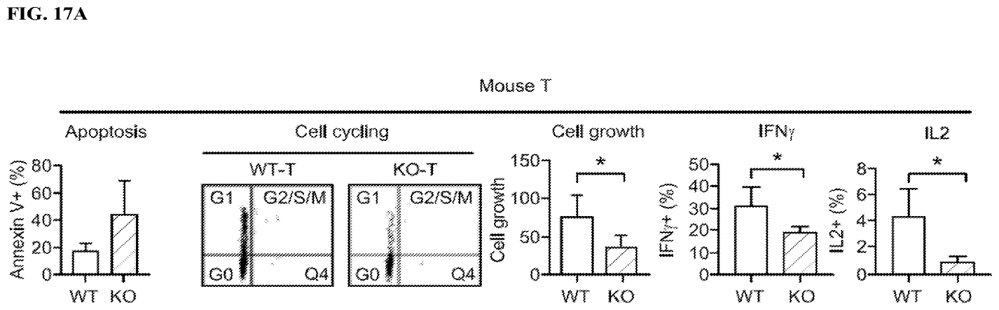

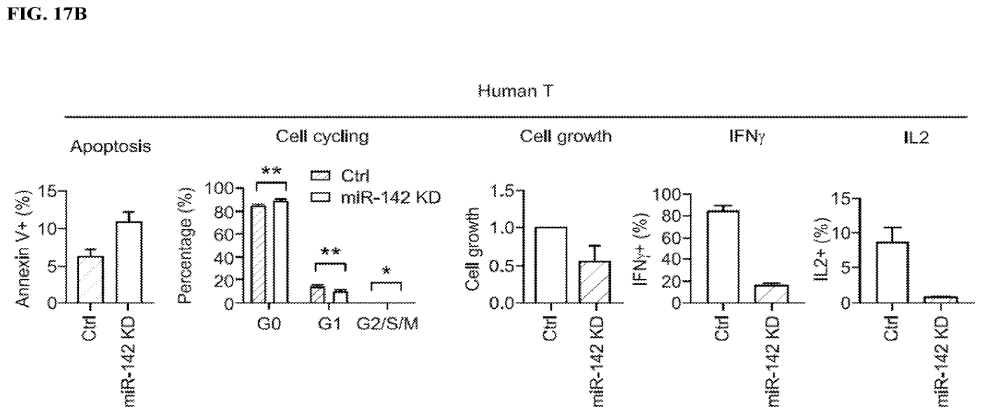

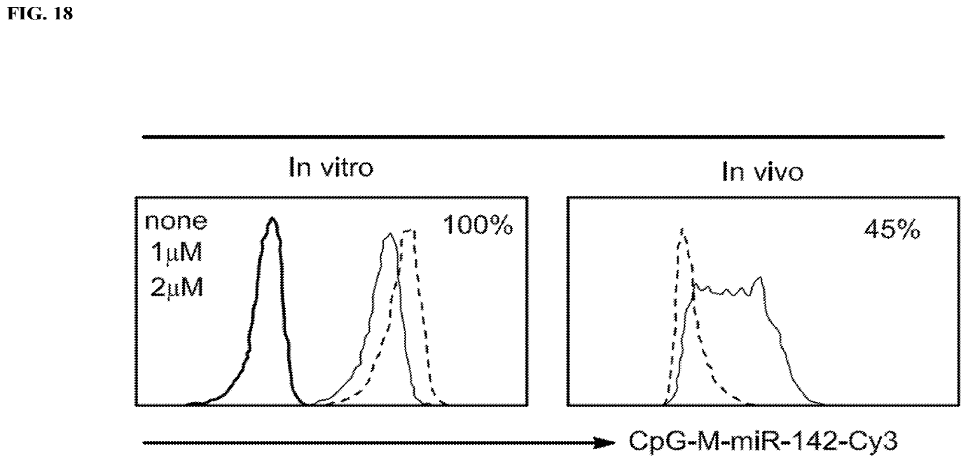

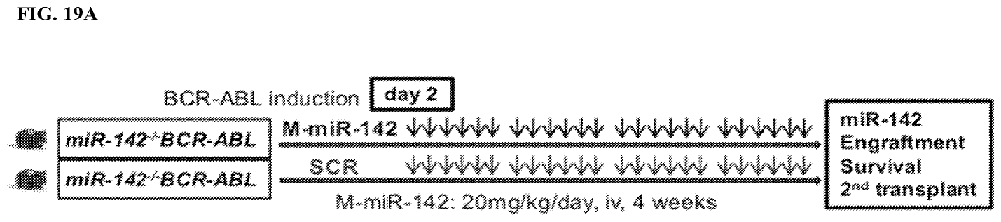

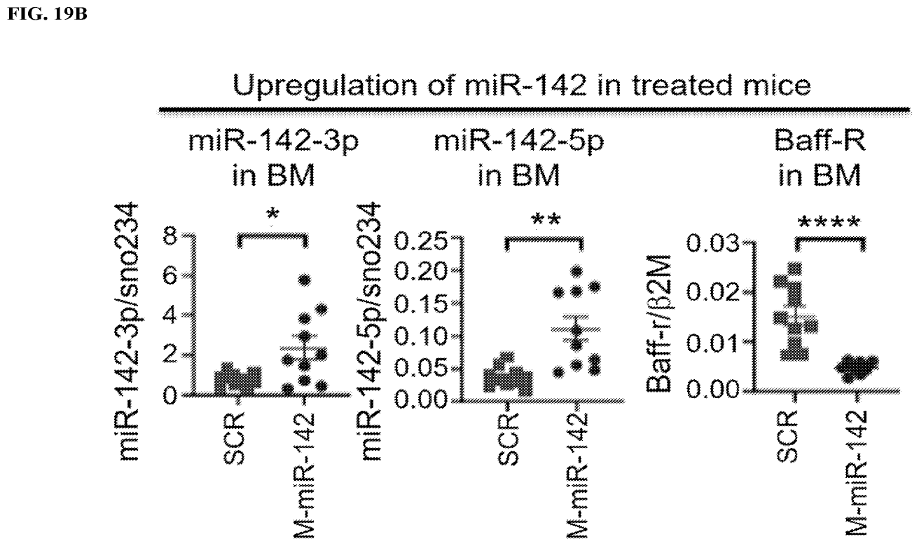

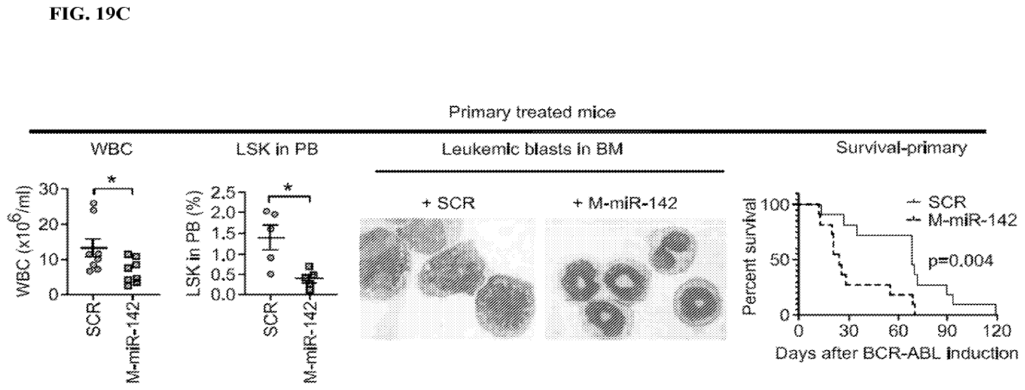

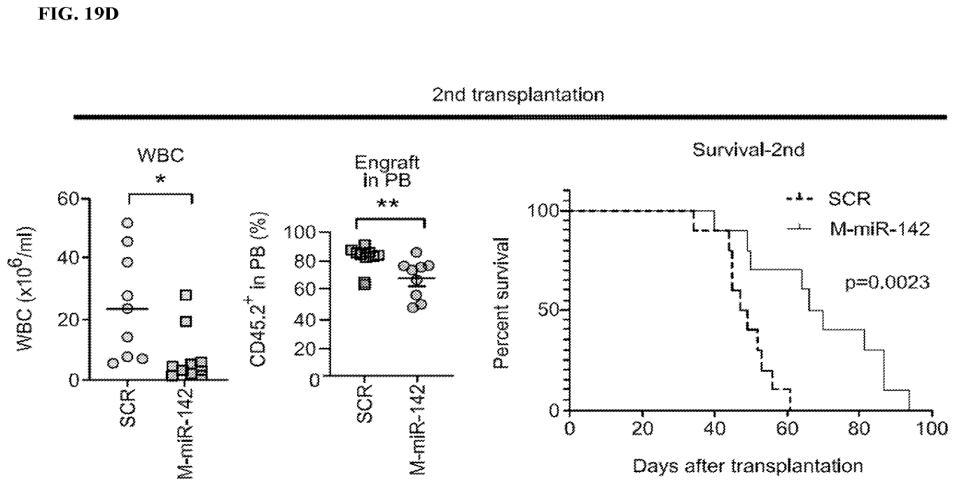

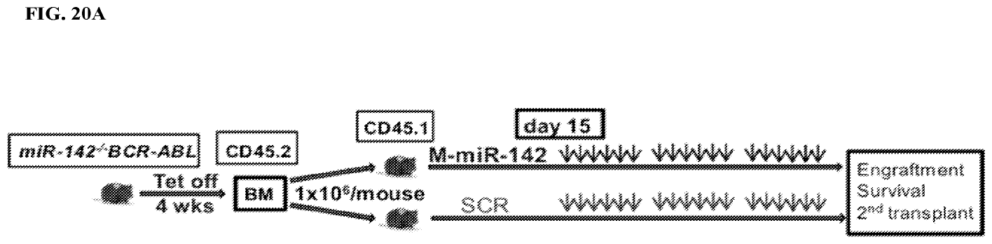

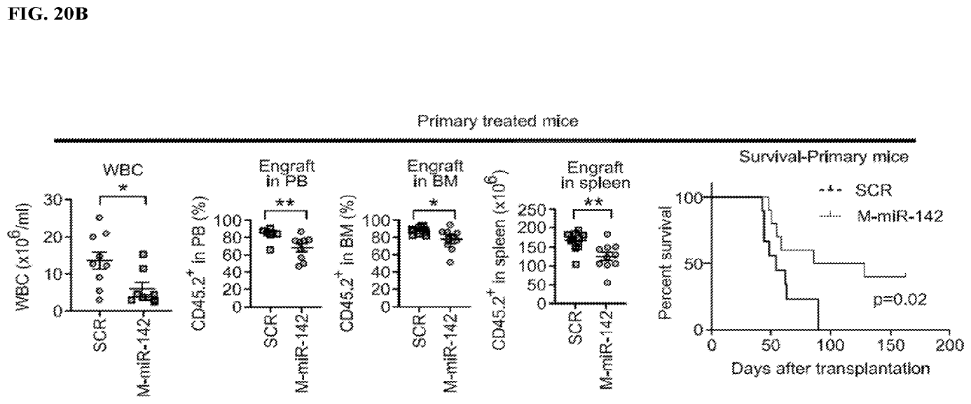

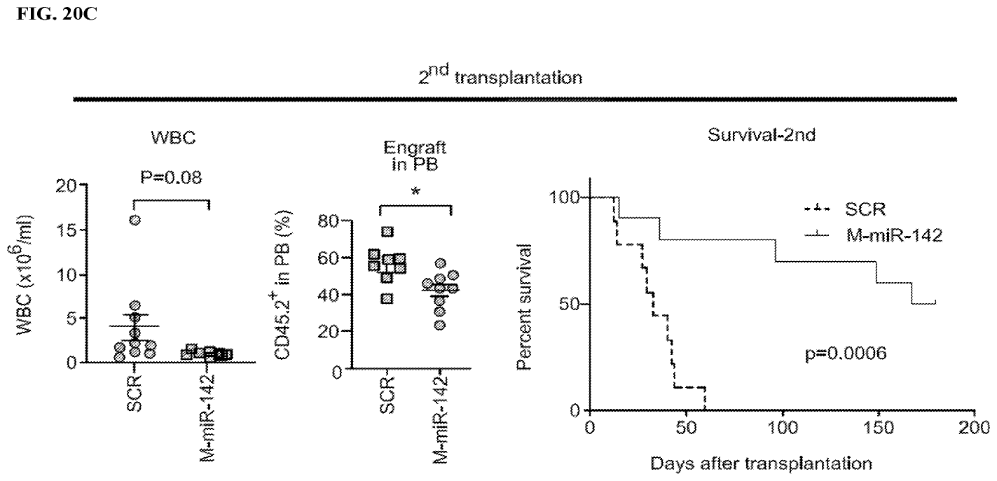

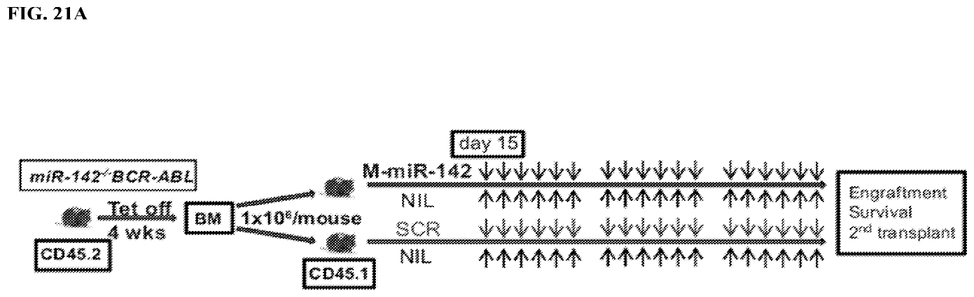

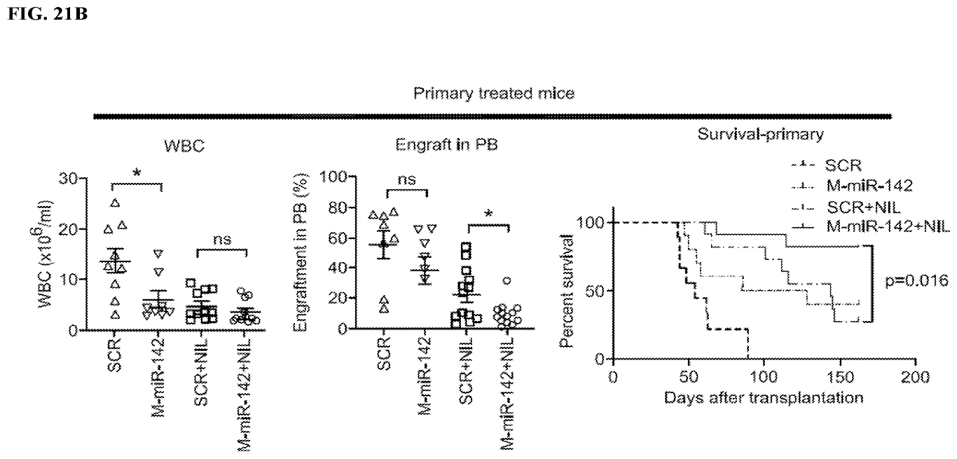

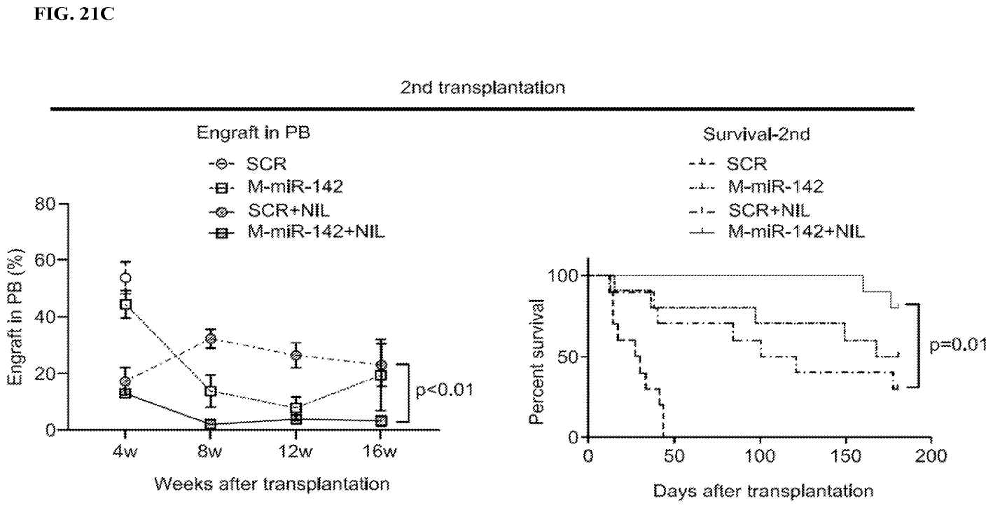

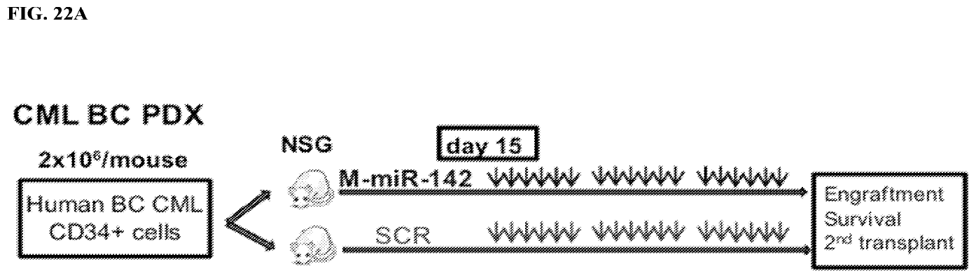

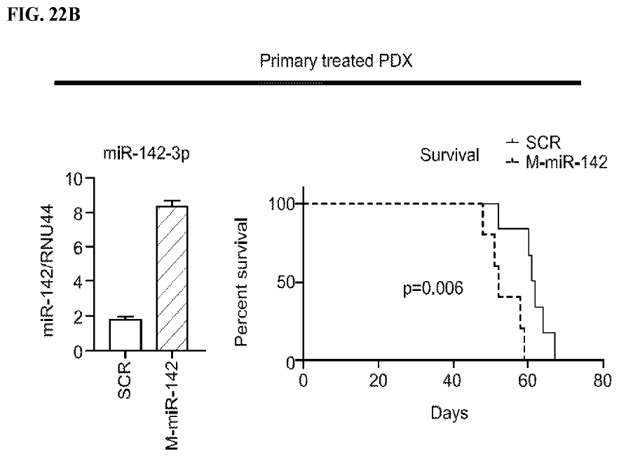

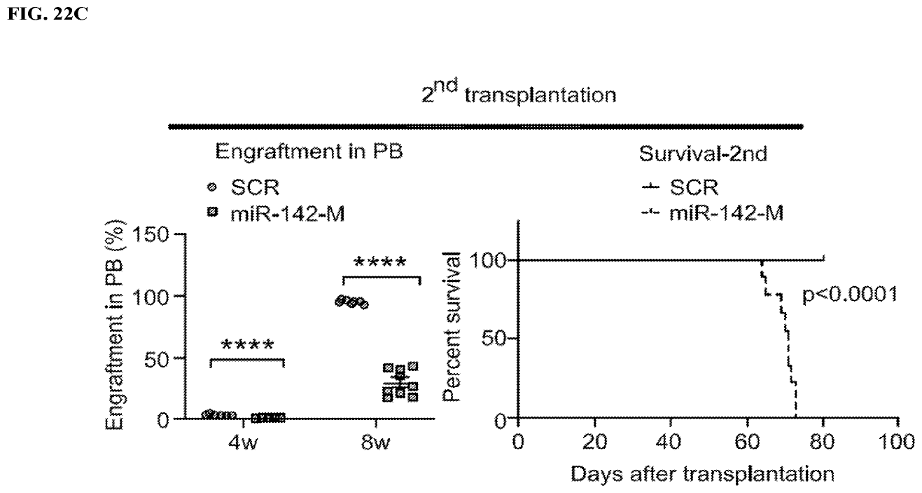

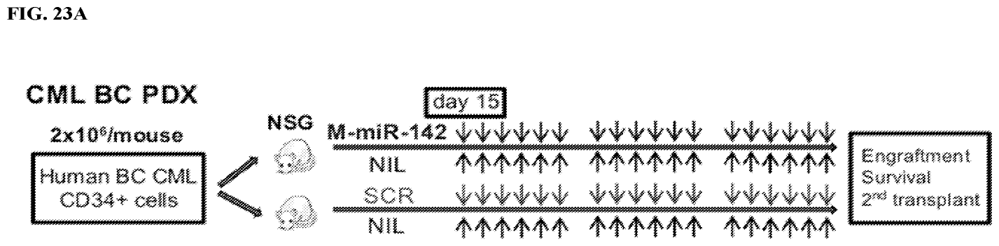

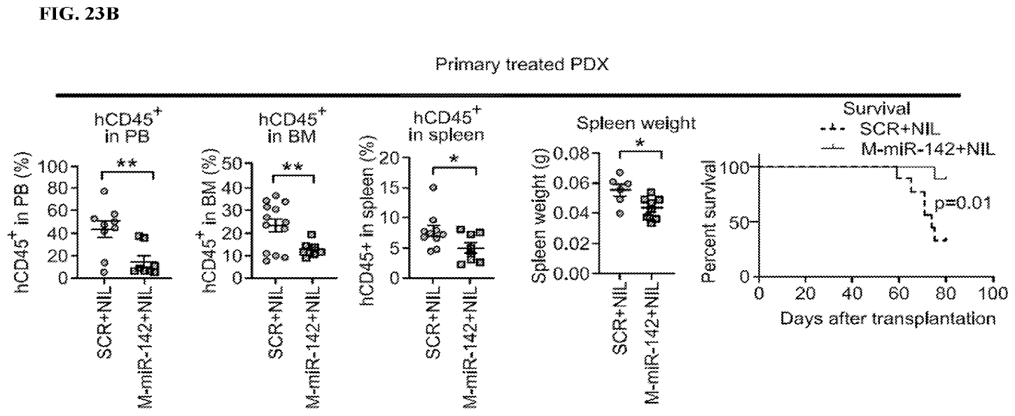

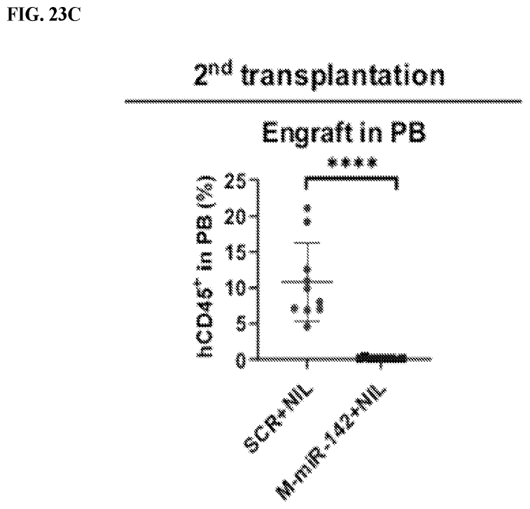

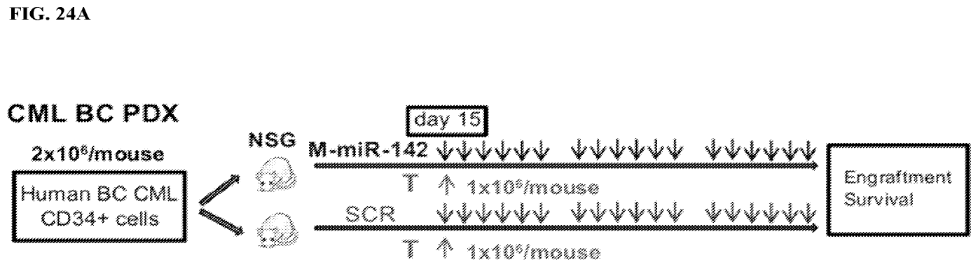

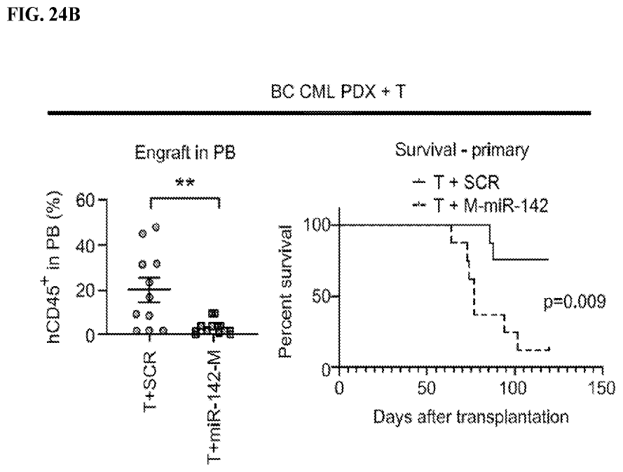

In embodiments, the disclosure provides the compound shown in FIG. 15. In embodiments of the compound shown in FIG. 15, each “o” is:

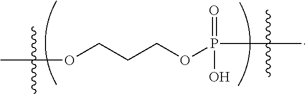

In embodiments, the compound further comprises a detectable moiety. The detectable moiety can be any known in the art and described herein. In embodiments, the detectable moiety is an enzyme, biotin, digoxigenin, a paramagnetic molecule, a contrast agent, gadolinium, a radioisotope, radionuclide, fluorodeoxyglucose, barium sulfate, thorium dioxide, gold, a fluorophore, a hapten, a protein, a fluorescent moiety, or a combination of two or more thereof. In embodiments, the contrast agent is a magnetic resonance imaging contrast agent, an X-ray contrast agent, or an iodinated contrast agent. In embodiments, the detectable agent is a fluorophore (e.g., fluorescein, rhodamine, coumarin, cyanine, or analogs thereof). In embodiments, the detectable agent is a chemiluminescent agent. In embodiments, the detectable agent is a radionuclide. In embodiments, the detectable agent is a radioisotope. In embodiments, the detectable agent is a paramagnetic molecule or a paramagnetic nanoparticle. The detectable moiety can be bonded to R¹, R², or L¹.

### Pharmaceutical Compositions

In embodiments, the disclosure provides pharmaceutical compositions comprising a pharmaceutically acceptable excipient and an effective amount of the compounds described herein, including all embodiments thereof. In embodiments, the disclosure provides pharmaceutical compositions comprising a pharmaceutically acceptable excipient and a therapeutically effective amount of the hybridized nucleic acids, as described herein, including all embodiments thereof.

A “effective amount” is an amount sufficient for a compound of the disclosure to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound (e.g., neural stem cells, vesicles) that are capable of achieving the methods described herein, as measured using the methods described herein or known in the art.

As is known in the art, therapeutically effective amounts for use in humans can also be determined from animal models. For example, a dose for humans can be formulated to achieve a concentration that has been found to be effective in animals. The dosage in humans can be adjusted by monitoring the effectiveness of the compositions, neural stem cells, and vesicles described herein, and adjusting the dosage upwards or downwards. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent (e.g., compounds, hybridized nucleic acids) sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intra-tumoral, intravenous, intramuscular, intra-arteriole, intradermal, subcutaneous, intraperitoneal, intraventricular, and intracranial. In embodiments, the neural stem cells, vesicles or pharmaceutical compositions described herein are parenterally administered to a patient. In embodiments, the neural stem cells, vesicles or pharmaceutical compositions described herein are administered intra-tumorally to a patient. Other modes of delivery include, but are not limited to, the use of liposomal formulations, intravenous infusion, transdermal patches, etc. In embodiments, the administering does not include administration of any active agent other than the recited active agent.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

### Dose and Dosing Regimens

The dosage and frequency (single or multiple doses) of the active agents described herein, including all embodiments thereof, administered to a subject can vary depending upon a variety of factors, for example, whether the mammal suffers from another disease, and its route of administration; size, age, sex, health, body weight, body mass index, and diet of the recipient; nature and extent of symptoms of the disease being treated (e.g. symptoms of cancer and severity of such symptoms), kind of concurrent treatment, complications from the disease being treated or other health-related problems. Other therapeutic regimens or agents can be used in conjunction with the methods described herein. Adjustment and manipulation of established dosages (e.g., frequency and duration) are well within the ability of those skilled in the art.

For any active agents described herein, the effective amount can be initially determined from cell culture assays. Target concentrations will be those concentrations of active agents that are capable of achieving the methods described herein, as measured using the methods described herein or known in the art. As is known in the art, effective amounts of active agents for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

Dosages of the active agents may be varied depending upon the requirements of the patient. The dose administered to a patient should be sufficient to affect a beneficial therapeutic response in the patient over time. The size of the dose also will be determined by the existence, nature, and extent of any adverse side-effects. Determination of the proper dosage for a particular situation is within the skill of the art. Generally, treatment is initiated with smaller dosages which are less than the optimum dose of the active agents. Thereafter, the dosage is increased by small increments until the optimum effect under circumstances is reached. Dosage amounts and intervals can be adjusted individually to provide levels of the active agents effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active agents by considering factors such as compound potency, relative bioavailability, patient body weight, presence and severity of adverse side effects.

In embodiments, the active agent is administered to a patient at an amount of about 0.01 mg/kg to about 500 mg/kg. In aspects, the active agent is administered to a patient in an amount of about 0.1 mg/kg, 0.5 mg/kg, 1 mg/kg, 2 mg/kg, 3 mg/kg, 4 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg, or 300 mg/kg. It is understood that where the amount is referred to as “mg/kg,” the amount is milligram per kilogram body weight of the subject being administered with the active agents. In aspects, the active agent is administered to a patient in an amount from about 0.1 mg to about 1,000 mg per day, as a single dose, or in a dose administered two or three times per day.

### Methods of Treatment

The disclosure provides methods of treating myeloid leukemia in a patient in need thereof by administering to the patient an effective amount of the compound of Formula (I) described herein, including any embodiment thereof. In embodiments, the methods comprise administering to the patient an effective amount of a pharmaceutical composition comprising a pharmaceutically acceptable excipient and the compound of Formula (I) described herein, including any embodiment thereof. In embodiments, a biological sample obtained from the patient has reduced miR-142 levels relative to a control. In embodiments, the reduced miR-142 levels are in CD34⁺ cells, CD38⁻ cells, or both CD34⁺ cells and CD38⁻ cells. In embodiments, the myeloid leukemia is chronic myeloid leukemia, chronic phase of chronic myeloid leukemia, accelerated phase of chronic myeloid leukemia, blast phase of chronic myeloid leukemia, acute myeloid leukemia, secondary acute myeloid leukemia, secondary acute myeloid leukemia related to therapy, secondary acute myeloid leukemia related to an antecedent hematologic disorder (e.g., myelodysplastic syndrome, myeloproliferative neoplasm, or myelodysplastic syndrome/myeloproliferative neoplasm overlap syndrome). In embodiments, the methods provide increased expression of miR-142. In embodiments, the methods provide increased expression of miR-142-3p and miR-142-5p. In embodiments, the methods provide reduced expression of the miR-142 target Baff-R in bone marrow cells. In embodiments, the myeloid leukemia is resistant to a tyrosine kinase inhibitor. In embodiments, the methods further comprising administering to the patient an effective amount of a tyrosine kinase inhibitor. In embodiments, the methods further comprising administering to the patient an effective amount of a tyrosine kinase inhibitor selected from the group consisting of imatinib, dasatinib, nilotinib, bosutinib, and ponatinib. In embodiments, the methods further comprising administering to the patient an effective amount of a tyrosine kinase inhibitor selected from the group consisting of imatinib, dasatinib, nilotinib, bosutinib, ponatinib, axitinib, crizotinib, erlotinib, gefitinib, lapatinib, pazopanib, ruxolitinib, sunitinib, and vemurafenib. In embodiments, the tyrosine kinase inhibitor is imatinib. In embodiments, the tyrosine kinase inhibitor is dasatinib. In embodiments, the tyrosine kinase inhibitor is nilotinib. In embodiments, the tyrosine kinase inhibitor is bosutinib. In embodiments, the tyrosine kinase inhibitor is ponatinib. In embodiments, the tyrosine kinase inhibitor is axitinib. In embodiments, the tyrosine kinase inhibitor is crizotinib. In embodiments, the tyrosine kinase inhibitor is erlotinib. In embodiments, the tyrosine kinase inhibitor is gefitinib. In embodiments, the tyrosine kinase inhibitor is lapatinib. In embodiments, the tyrosine kinase inhibitor is pazopanib. In embodiments, the tyrosine kinase inhibitor is ruxolitinib. In embodiments, the tyrosine kinase inhibitor is sunitinib. In embodiments, the tyrosine kinase inhibitor is vemurafenib.

The disclosure provides methods of treating myeloid leukemia in a patient in need thereof by detecting reduced miR-142 levels relative to a control in a biological sample obtained from the patient, and administering to the patient an effective amount of the compound of Formula (I) described herein, including any embodiment thereof. In embodiments, the methods comprise administering to the patient an effective amount of a pharmaceutical composition comprising a pharmaceutically acceptable excipient and the compound of Formula (I) described herein, including any embodiment thereof. In embodiments, the reduced miR-142 levels are in CD34⁺ cells, CD38⁻ cells, or both CD34⁺ cells and CD38⁻ cells. In embodiments, the myeloid leukemia is chronic myeloid leukemia, chronic phase of chronic myeloid leukemia, accelerated phase of chronic myeloid leukemia, blast phase of chronic myeloid leukemia, acute myeloid leukemia, secondary acute myeloid leukemia, secondary acute myeloid leukemia related to therapy, secondary acute myeloid leukemia related to an antecedent hematologic disorder (e.g., myelodysplastic syndrome, myeloproliferative neoplasm, or myelodysplastic syndrome/myeloproliferative neoplasm overlap syndrome). In embodiments, the methods provide increased expression of miR-142. In embodiments, the methods provide increased expression of miR-142-3p and miR-142-5p. In embodiments, the methods provide reduced expression of the miR-142 target Baff-R in bone marrow cells. In embodiments, the myeloid leukemia is resistant to a tyrosine kinase inhibitor. In embodiments, the methods further comprising administering to the patient an effective amount of a tyrosine kinase inhibitor. In embodiments, the methods further comprising administering to the patient an effective amount of a tyrosine kinase inhibitor selected from the group consisting of imatinib, dasatinib, nilotinib, bosutinib, and ponatinib. In embodiments, the methods further comprising administering to the patient an effective amount of a tyrosine kinase inhibitor selected from the group consisting of imatinib, dasatinib, nilotinib, bosutinib, ponatinib, axitinib, crizotinib, erlotinib, gefitinib, lapatinib, pazopanib, ruxolitinib, sunitinib, and vemurafenib. In embodiments, the tyrosine kinase inhibitor is imatinib. In embodiments, the tyrosine kinase inhibitor is dasatinib. In embodiments, the tyrosine kinase inhibitor is nilotinib. In embodiments, the tyrosine kinase inhibitor is bosutinib. In embodiments, the tyrosine kinase inhibitor is ponatinib. In embodiments, the tyrosine kinase inhibitor is axitinib. In embodiments, the tyrosine kinase inhibitor is crizotinib. In embodiments, the tyrosine kinase inhibitor is erlotinib. In embodiments, the tyrosine kinase inhibitor is gefitinib. In embodiments, the tyrosine kinase inhibitor is lapatinib. In embodiments, the tyrosine kinase inhibitor is pazopanib. In embodiments, the tyrosine kinase inhibitor is ruxolitinib. In embodiments, the tyrosine kinase inhibitor is sunitinib. In embodiments, the tyrosine kinase inhibitor is vemurafenib.

The disclosure provides methods of treating myeloid leukemia in a patient in need thereof, the method comprising: (i) measuring miR-142 levels in a biological sample obtained from the patient; (ii) identifying the patient as having myeloid leukemia when the miR-142 levels are reduced relative to a control; and (iii) administering to the patient identified as having myeloid leukemia based on the reduced miR-142 levels an effective amount of the compound of Formula (I) described herein, including any embodiment thereof. In embodiments, the reduced miR-142 levels are in CD34⁺ cells, CD38⁻ cells, or both CD34⁺ cells and CD38⁻ cells. In embodiments, the myeloid leukemia is chronic myeloid leukemia, chronic phase of chronic myeloid leukemia, accelerated phase of chronic myeloid leukemia, blast phase of chronic myeloid leukemia, acute myeloid leukemia, secondary acute myeloid leukemia, secondary acute myeloid leukemia related to therapy, secondary acute myeloid leukemia related to an antecedent hematologic disorder (e.g., myelodysplastic syndrome, myeloproliferative neoplasm, or myelodysplastic syndrome/myeloproliferative neoplasm overlap syndrome). In embodiments, the methods provide increased expression of miR-142. In embodiments, the methods provide increased expression of miR-142-3p and miR-142-5p. In embodiments, the methods provide reduced expression of the miR-142 target Baff-R in bone marrow cells. In embodiments, the myeloid leukemia is resistant to a tyrosine kinase inhibitor. In embodiments, the methods further comprising administering to the patient an effective amount of a tyrosine kinase inhibitor. In embodiments, the methods further comprising administering to the patient an effective amount of a tyrosine kinase inhibitor selected from the group consisting of imatinib, dasatinib, nilotinib, bosutinib, and ponatinib. In embodiments, the methods further comprising administering to the patient an effective amount of a tyrosine kinase inhibitor selected from the group consisting of imatinib, dasatinib, nilotinib, bosutinib, ponatinib, axitinib, crizotinib, erlotinib, gefitinib, lapatinib, pazopanib, ruxolitinib, sunitinib, and vemurafenib. In embodiments, the tyrosine kinase inhibitor is imatinib. In embodiments, the tyrosine kinase inhibitor is dasatinib. In embodiments, the tyrosine kinase inhibitor is nilotinib. In embodiments, the tyrosine kinase inhibitor is bosutinib. In embodiments, the tyrosine kinase inhibitor is ponatinib. In embodiments, the tyrosine kinase inhibitor is axitinib. In embodiments, the tyrosine kinase inhibitor is crizotinib. In embodiments, the tyrosine kinase inhibitor is erlotinib. In embodiments, the tyrosine kinase inhibitor is gefitinib. In embodiments, the tyrosine kinase inhibitor is lapatinib. In embodiments, the tyrosine kinase inhibitor is pazopanib. In embodiments, the tyrosine kinase inhibitor is ruxolitinib. In embodiments, the tyrosine kinase inhibitor is sunitinib. In embodiments, the tyrosine kinase inhibitor is vemurafenib.

The disclosure provides methods of treating chronic myeloid leukemia in a patient in need thereof by administering to the patient an effective amount of the compound of Formula (I) described herein, including any embodiment thereof. In embodiments, the methods comprise administering to the patient an effective amount of a pharmaceutical composition comprising a pharmaceutically acceptable excipient and the compound of Formula (I) described herein, including any embodiment thereof. In embodiments, the chronic myeloid leukemia is the chronic phase of chronic myeloid leukemia. In embodiments, the chronic myeloid leukemia is the accelerated phase of chronic myeloid leukemia. In embodiments, the chronic myeloid leukemia is the blast phase of chronic myeloid leukemia. In embodiments, a biological sample obtained from the patient has reduced miR-142 levels relative to a control. In embodiments, the reduced miR-142 levels are in CD34⁺ cells, CD38⁻ cells, or both CD34⁺ cells and CD38⁻ cells. In embodiments, the methods provide increased expression of miR-142. In embodiments, the methods provide increased expression of miR-142-3p and miR-142-5p. In embodiments, the methods provide reduced expression of the miR-142 target Baff-R in bone marrow cells. In embodiments, the myeloid leukemia is resistant to a tyrosine kinase inhibitor. In embodiments, the methods further comprising administering to the patient an effective amount of a tyrosine kinase inhibitor. In embodiments, the methods further comprising administering to the patient an effective amount of a tyrosine kinase inhibitor selected from the group consisting of imatinib, dasatinib, nilotinib, bosutinib, and ponatinib. In embodiments, the methods further comprising administering to the patient an effective amount of a tyrosine kinase inhibitor selected from the group consisting of imatinib, dasatinib, nilotinib, bosutinib, ponatinib, axitinib, crizotinib, erlotinib, gefitinib, lapatinib, pazopanib, ruxolitinib, sunitinib, and vemurafenib. In embodiments, the tyrosine kinase inhibitor is imatinib. In embodiments, the tyrosine kinase inhibitor is dasatinib. In embodiments, the tyrosine kinase inhibitor is nilotinib. In embodiments, the tyrosine kinase inhibitor is bosutinib. In embodiments, the tyrosine kinase inhibitor is ponatinib. In embodiments, the tyrosine kinase inhibitor is axitinib. In embodiments, the tyrosine kinase inhibitor is crizotinib. In embodiments, the tyrosine kinase inhibitor is erlotinib. In embodiments, the tyrosine kinase inhibitor is gefitinib. In embodiments, the tyrosine kinase inhibitor is lapatinib. In embodiments, the tyrosine kinase inhibitor is pazopanib. In embodiments, the tyrosine kinase inhibitor is ruxolitinib. In embodiments, the tyrosine kinase inhibitor is sunitinib. In embodiments, the tyrosine kinase inhibitor is vemurafenib.

The disclosure provides methods of treating chronic myeloid leukemia in a patient in need thereof by detecting reduced miR-142 levels relative to a control in a biological sample obtained from the patient, and administering to the patient an effective amount of the compound of Formula (I) described herein, including any embodiment thereof. In embodiments, the methods comprise administering to the patient an effective amount of a pharmaceutical composition comprising a pharmaceutically acceptable excipient and the compound of Formula (I) described herein, including any embodiment thereof. In embodiments, the chronic myeloid leukemia is the chronic phase of chronic myeloid leukemia. In embodiments, the chronic myeloid leukemia is the accelerated phase of chronic myeloid leukemia. In embodiments, the chronic myeloid leukemia is the blast phase of chronic myeloid leukemia. In embodiments, the reduced miR-142 levels are in CD34⁺ cells, CD38⁻ cells, or both CD34⁺ cells and CD38⁻ cells. In embodiments, the methods provide increased expression of miR-142. In embodiments, the methods provide increased expression of miR-142-3p and miR-142-5p. In embodiments, the methods provide reduced expression of the miR-142 target Baff-R in bone marrow cells. In embodiments, the myeloid leukemia is resistant to a tyrosine kinase inhibitor. In embodiments, the methods further comprising administering to the patient an effective amount of a tyrosine kinase inhibitor. In embodiments, the methods further comprising administering to the patient an effective amount of a tyrosine kinase inhibitor selected from the group consisting of imatinib, dasatinib, nilotinib, bosutinib, and ponatinib. In embodiments, the methods further comprising administering to the patient an effective amount of a tyrosine kinase inhibitor selected from the group consisting of imatinib, dasatinib, nilotinib, bosutinib, ponatinib, axitinib, crizotinib, erlotinib, gefitinib, lapatinib, pazopanib, ruxolitinib, sunitinib, and vemurafenib. In embodiments, the tyrosine kinase inhibitor is imatinib. In embodiments, the tyrosine kinase inhibitor is dasatinib. In embodiments, the tyrosine kinase inhibitor is nilotinib. In embodiments, the tyrosine kinase inhibitor is bosutinib. In embodiments, the tyrosine kinase inhibitor is ponatinib. In embodiments, the tyrosine kinase inhibitor is axitinib. In embodiments, the tyrosine kinase inhibitor is crizotinib. In embodiments, the tyrosine kinase inhibitor is erlotinib. In embodiments, the tyrosine kinase inhibitor is gefitinib. In embodiments, the tyrosine kinase inhibitor is lapatinib. In embodiments, the tyrosine kinase inhibitor is pazopanib. In embodiments, the tyrosine kinase inhibitor is ruxolitinib. In embodiments, the tyrosine kinase inhibitor is sunitinib. In embodiments, the tyrosine kinase inhibitor is vemurafenib.

The disclosure provides methods of treating chronic myeloid leukemia in a patient in need thereof, the method comprising: (i) measuring miR-142 levels in a biological sample obtained from the patient; (ii) identifying the patient as having chronic myeloid leukemia when the miR-142 levels are reduced relative to a control; and (iii) administering to the patient identified as having chronic myeloid leukemia based on the reduced miR-142 levels an effective amount of the compound of Formula (I) described herein, including any embodiment thereof. In embodiments, the methods comprise administering to the patient identified as having chronic myeloid leukemia based on the reduced miR-142 levels an effective amount of a pharmaceutical composition comprising a pharmaceutically acceptable excipient and the compound of Formula (I) described herein, including any embodiment thereof. In embodiments, the chronic myeloid leukemia is the chronic phase of chronic myeloid leukemia. In embodiments, the chronic myeloid leukemia is the accelerated phase of chronic myeloid leukemia. In embodiments, the chronic myeloid leukemia is the blast phase of chronic myeloid leukemia. In embodiments, the reduced miR-142 levels are in CD34⁺ cells, CD38⁻ cells, or both CD34⁺ cells and CD38⁻ cells. In embodiments, the methods provide increased expression of miR-142. In embodiments, the methods provide increased expression of miR-142-3p and miR-142-5p. In embodiments, the methods provide reduced expression of the miR-142 target Baff-R in bone marrow cells. In embodiments, the myeloid leukemia is resistant to a tyrosine kinase inhibitor. In embodiments, the methods further comprising administering to the patient an effective amount of a tyrosine kinase inhibitor. In embodiments, the methods further comprising administering to the patient an effective amount of a tyrosine kinase inhibitor selected from the group consisting of imatinib, dasatinib, nilotinib, bosutinib, and ponatinib. In embodiments, the methods further comprising administering to the patient an effective amount of a tyrosine kinase inhibitor selected from the group consisting of imatinib, dasatinib, nilotinib, bosutinib, ponatinib, axitinib, crizotinib, erlotinib, gefitinib, lapatinib, pazopanib, ruxolitinib, sunitinib, and vemurafenib. In embodiments, the tyrosine kinase inhibitor is imatinib. In embodiments, the tyrosine kinase inhibitor is dasatinib. In embodiments, the tyrosine kinase inhibitor is nilotinib. In embodiments, the tyrosine kinase inhibitor is bosutinib. In embodiments, the tyrosine kinase inhibitor is ponatinib. In embodiments, the tyrosine kinase inhibitor is axitinib. In embodiments, the tyrosine kinase inhibitor is crizotinib. In embodiments, the tyrosine kinase inhibitor is erlotinib. In embodiments, the tyrosine kinase inhibitor is gefitinib. In embodiments, the tyrosine kinase inhibitor is lapatinib. In embodiments, the tyrosine kinase inhibitor is pazopanib. In embodiments, the tyrosine kinase inhibitor is ruxolitinib. In embodiments, the tyrosine kinase inhibitor is sunitinib. In embodiments, the tyrosine kinase inhibitor is vemurafenib.

The disclosure provides methods of treating acute myeloid leukemia in a patient in need thereof by administering to the patient an effective amount of the compound of Formula (I) described herein, including any embodiment thereof. In embodiments, the methods comprise administering to the patient an effective amount of a pharmaceutical composition comprising a pharmaceutically acceptable excipient and the compound of Formula (I) described herein, including any embodiment thereof. In embodiments, the acute myeloid leukemia is secondary acute myeloid leukemia. In embodiments, the secondary acute myeloid leukemia is related to therapy. In embodiments, the secondary acute myeloid leukemia is related to an antecedent hematologic disorder. In embodiments, the antecedent hematologic disorder is myelodysplastic syndrome, myeloproliferative neoplasm, or myelodysplastic syndrome/myeloproliferative neoplasm overlap syndrome. In embodiments, a biological sample obtained from the patient has reduced miR-142 levels relative to a control. In embodiments, the reduced miR-142 levels are in CD34⁺ cells, CD38⁻ cells, or both CD34⁺ cells and CD38⁻ cells. In embodiments, the methods provide increased expression of miR-142. In embodiments, the methods provide increased expression of miR-142-3p and miR-142-5p. In embodiments, the methods provide reduced expression of the miR-142 target Baff-R in bone marrow cells. In embodiments, the myeloid leukemia is resistant to a tyrosine kinase inhibitor. In embodiments, the methods further comprising administering to the patient an effective amount of a tyrosine kinase inhibitor. In embodiments, the methods further comprising administering to the patient an effective amount of a tyrosine kinase inhibitor selected from the group consisting of imatinib, dasatinib, nilotinib, bosutinib, and ponatinib. In embodiments, the methods further comprising administering to the patient an effective amount of a tyrosine kinase inhibitor selected from the group consisting of imatinib, dasatinib, nilotinib, bosutinib, ponatinib, axitinib, crizotinib, erlotinib, gefitinib, lapatinib, pazopanib, ruxolitinib, sunitinib, and vemurafenib. In embodiments, the tyrosine kinase inhibitor is imatinib. In embodiments, the tyrosine kinase inhibitor is dasatinib. In embodiments, the tyrosine kinase inhibitor is nilotinib. In embodiments, the tyrosine kinase inhibitor is bosutinib. In embodiments, the tyrosine kinase inhibitor is ponatinib. In embodiments, the tyrosine kinase inhibitor is axitinib. In embodiments, the tyrosine kinase inhibitor is crizotinib. In embodiments, the tyrosine kinase inhibitor is erlotinib. In embodiments, the tyrosine kinase inhibitor is gefitinib. In embodiments, the tyrosine kinase inhibitor is lapatinib. In embodiments, the tyrosine kinase inhibitor is pazopanib. In embodiments, the tyrosine kinase inhibitor is ruxolitinib. In embodiments, the tyrosine kinase inhibitor is sunitinib. In embodiments, the tyrosine kinase inhibitor is vemurafenib.

The disclosure provides methods of treating acute myeloid leukemia in a patient in need thereof by detecting reduced miR-142 levels relative to a control in a biological sample obtained from the patient, and administering to the patient an effective amount of the compound of Formula (I) described herein, including any embodiment thereof. In embodiments, the methods comprise administering to the patient an effective amount of a pharmaceutical composition comprising a pharmaceutically acceptable excipient and the compound of Formula (I) described herein, including any embodiment thereof. In embodiments, the acute myeloid leukemia is secondary acute myeloid leukemia. In embodiments, the secondary acute myeloid leukemia is related to therapy. In embodiments, the secondary acute myeloid leukemia is related to an antecedent hematologic disorder. In embodiments, the antecedent hematologic disorder is myelodysplastic syndrome, myeloproliferative neoplasm, or myelodysplastic syndrome/myeloproliferative neoplasm overlap syndrome. In embodiments, the reduced miR-142 levels are in CD34⁺ cells, CD38⁻ cells, or both CD34⁺ cells and CD38⁻ cells. In embodiments, the methods provide increased expression of miR-142. In embodiments, the methods provide increased expression of miR-142-3p and miR-142-5p. In embodiments, the methods provide reduced expression of the miR-142 target Baff-R in bone marrow cells. In embodiments, the myeloid leukemia is resistant to a tyrosine kinase inhibitor. In embodiments, the methods further comprising administering to the patient an effective amount of a tyrosine kinase inhibitor. In embodiments, the methods further comprising administering to the patient an effective amount of a tyrosine kinase inhibitor selected from the group consisting of imatinib, dasatinib, nilotinib, bosutinib, and ponatinib. In embodiments, the methods further comprising administering to the patient an effective amount of a tyrosine kinase inhibitor selected from the group consisting of imatinib, dasatinib, nilotinib, bosutinib, ponatinib, axitinib, crizotinib, erlotinib, gefitinib, lapatinib, pazopanib, ruxolitinib, sunitinib, and vemurafenib. In embodiments, the tyrosine kinase inhibitor is imatinib. In embodiments, the tyrosine kinase inhibitor is dasatinib. In embodiments, the tyrosine kinase inhibitor is nilotinib. In embodiments, the tyrosine kinase inhibitor is bosutinib. In embodiments, the tyrosine kinase inhibitor is ponatinib. In embodiments, the tyrosine kinase inhibitor is axitinib. In embodiments, the tyrosine kinase inhibitor is crizotinib. In embodiments, the tyrosine kinase inhibitor is erlotinib. In embodiments, the tyrosine kinase inhibitor is gefitinib. In embodiments, the tyrosine kinase inhibitor is lapatinib. In embodiments, the tyrosine kinase inhibitor is pazopanib. In embodiments, the tyrosine kinase inhibitor is ruxolitinib. In embodiments, the tyrosine kinase inhibitor is sunitinib. In embodiments, the tyrosine kinase inhibitor is vemurafenib.

The disclosure provides methods of treating myelodysplastic syndrome, myeloproliferative neoplasm, or myelodysplastic syndrome/myeloproliferative neoplasm overlap syndrome in a patient in need thereof by administering to the patient an effective amount of the compound of Formula (I) described herein, including any embodiment thereof. In embodiments, the methods comprise administering to the patient an effective amount of a pharmaceutical composition comprising a pharmaceutically acceptable excipient and the compound of Formula (I) described herein, including any embodiment thereof. In embodiments, the methods are for treating myelodysplastic syndrome. In embodiments, the methods are for treating myeloproliferative neoplasm. In embodiments, the methods are for treating myelodysplastic syndrome/myeloproliferative neoplasm overlap syndrome. In embodiments, a biological sample obtained from the patient has reduced miR-142 levels relative to a control. In embodiments, the reduced miR-142 levels are in CD34⁺ cells, CD38⁻ cells, or both CD34⁺ cells and CD38⁻ cells. In embodiments, the methods provide increased expression of miR-142. In embodiments, the methods provide increased expression of miR-142-3p and miR-142-5p. In embodiments, the methods provide reduced expression of the miR-142 target Baff-R in bone marrow cells. In embodiments, the myeloid leukemia is resistant to a tyrosine kinase inhibitor. In embodiments, the methods further comprising administering to the patient an effective amount of a tyrosine kinase inhibitor. In embodiments, the methods further comprising administering to the patient an effective amount of a tyrosine kinase inhibitor selected from the group consisting of imatinib, dasatinib, nilotinib, bosutinib, and ponatinib. In embodiments, the methods further comprising administering to the patient an effective amount of a tyrosine kinase inhibitor selected from the group consisting of imatinib, dasatinib, nilotinib, bosutinib, ponatinib, axitinib, crizotinib, erlotinib, gefitinib, lapatinib, pazopanib, ruxolitinib, sunitinib, and vemurafenib. In embodiments, the tyrosine kinase inhibitor is imatinib. In embodiments, the tyrosine kinase inhibitor is dasatinib. In embodiments, the tyrosine kinase inhibitor is nilotinib. In embodiments, the tyrosine kinase inhibitor is bosutinib. In embodiments, the tyrosine kinase inhibitor is ponatinib. In embodiments, the tyrosine kinase inhibitor is axitinib. In embodiments, the tyrosine kinase inhibitor is crizotinib. In embodiments, the tyrosine kinase inhibitor is erlotinib. In embodiments, the tyrosine kinase inhibitor is gefitinib. In embodiments, the tyrosine kinase inhibitor is lapatinib. In embodiments, the tyrosine kinase inhibitor is pazopanib. In embodiments, the tyrosine kinase inhibitor is ruxolitinib. In embodiments, the tyrosine kinase inhibitor is sunitinib. In embodiments, the tyrosine kinase inhibitor is vemurafenib.

The disclosure provides methods of treating myelodysplastic syndrome, myeloproliferative neoplasm, or myelodysplastic syndrome/myeloproliferative neoplasm overlap syndrome in a patient in need thereof by detecting reduced miR-142 levels relative to a control in a biological sample obtained from the patient, and administering to the patient an effective amount of the compound of Formula (I) described herein, including any embodiment thereof. In embodiments, the methods comprise administering to the patient an effective amount of a pharmaceutical composition comprising a pharmaceutically acceptable excipient and the compound of Formula (I) described herein, including any embodiment thereof. In embodiments, the methods are for treating myelodysplastic syndrome. In embodiments, the methods are for treating myeloproliferative neoplasm. In embodiments, the methods are for treating myelodysplastic syndrome/myeloproliferative neoplasm overlap syndrome. In embodiments, the reduced miR-142 levels are in CD34⁺ cells, CD38⁻ cells, or both CD34⁺ cells and CD38⁻ cells. In embodiments, the methods provide increased expression of miR-142. In embodiments, the methods provide increased expression of miR-142-3p and miR-142-5p. In embodiments, the methods provide reduced expression of the miR-142 target Baff-R in bone marrow cells. In embodiments, the myeloid leukemia is resistant to a tyrosine kinase inhibitor. In embodiments, the methods further comprising administering to the patient an effective amount of a tyrosine kinase inhibitor. In embodiments, the methods further comprising administering to the patient an effective amount of a tyrosine kinase inhibitor selected from the group consisting of imatinib, dasatinib, nilotinib, bosutinib, and ponatinib. In embodiments, the methods further comprising administering to the patient an effective amount of a tyrosine kinase inhibitor selected from the group consisting of imatinib, dasatinib, nilotinib, bosutinib, ponatinib, axitinib, crizotinib, erlotinib, gefitinib, lapatinib, pazopanib, ruxolitinib, sunitinib, and vemurafenib. In embodiments, the tyrosine kinase inhibitor is imatinib. In embodiments, the tyrosine kinase inhibitor is dasatinib. In embodiments, the tyrosine kinase inhibitor is nilotinib. In embodiments, the tyrosine kinase inhibitor is bosutinib. In embodiments, the tyrosine kinase inhibitor is ponatinib. In embodiments, the tyrosine kinase inhibitor is axitinib. In embodiments, the tyrosine kinase inhibitor is crizotinib. In embodiments, the tyrosine kinase inhibitor is erlotinib. In embodiments, the tyrosine kinase inhibitor is gefitinib. In embodiments, the tyrosine kinase inhibitor is lapatinib. In embodiments, the tyrosine kinase inhibitor is pazopanib. In embodiments, the tyrosine kinase inhibitor is ruxolitinib. In embodiments, the tyrosine kinase inhibitor is sunitinib. In embodiments, the tyrosine kinase inhibitor is vemurafenib.

The disclosure provides methods of treating myelodysplastic syndrome, myeloproliferative neoplasm, or myelodysplastic syndrome/myeloproliferative neoplasm overlap syndrome in a patient in need thereof, the method comprising: (i) measuring miR-142 levels in a biological sample obtained from the patient; (ii) identifying the patient as having myelodysplastic syndrome, myeloproliferative neoplasm, or myelodysplastic syndrome/myeloproliferative neoplasm overlap syndrome when the miR-142 levels are reduced relative to a control; and (iii) administering to the patient identified as having myelodysplastic syndrome, myeloproliferative neoplasm, or myelodysplastic syndrome/myeloproliferative neoplasm overlap syndrome based on the reduced miR-142 levels an effective amount of the compound of Formula (I) described herein, including any embodiment thereof. In embodiments, the methods comprise administering to the patient identified as having myelodysplastic syndrome, myeloproliferative neoplasm, or myelodysplastic syndrome/myeloproliferative neoplasm overlap syndrome based on the reduced miR-142 levels an effective amount of a pharmaceutical composition comprising a pharmaceutically acceptable excipient and the compound of Formula (I) described herein, including any embodiment thereof. In embodiments, the methods are for treating myelodysplastic syndrome. In embodiments, the methods are for treating myeloproliferative neoplasm. In embodiments, the methods are for treating myelodysplastic syndrome/myeloproliferative neoplasm overlap syndrome. In embodiments, the reduced miR-142 levels are in CD34⁺ cells, CD38⁻ cells, or both CD34⁺ cells and CD38⁻ cells. In embodiments, the methods provide increased expression of miR-142. In embodiments, the methods provide increased expression of miR-142-3p and miR-142-5p. In embodiments, the methods provide reduced expression of the miR-142 target Baff-R in bone marrow cells. In embodiments, the myeloid leukemia is resistant to a tyrosine kinase inhibitor. In embodiments, the methods further comprising administering to the patient an effective amount of a tyrosine kinase inhibitor. In embodiments, the methods further comprising administering to the patient an effective amount of a tyrosine kinase inhibitor selected from the group consisting of imatinib, dasatinib, nilotinib, bosutinib, and ponatinib. In embodiments, the methods further comprising administering to the patient an effective amount of a tyrosine kinase inhibitor selected from the group consisting of imatinib, dasatinib, nilotinib, bosutinib, ponatinib, axitinib, crizotinib, erlotinib, gefitinib, lapatinib, pazopanib, ruxolitinib, sunitinib, and vemurafenib. In embodiments, the tyrosine kinase inhibitor is imatinib. In embodiments, the tyrosine kinase inhibitor is dasatinib. In embodiments, the tyrosine kinase inhibitor is nilotinib. In embodiments, the tyrosine kinase inhibitor is bosutinib. In embodiments, the tyrosine kinase inhibitor is ponatinib. In embodiments, the tyrosine kinase inhibitor is axitinib. In embodiments, the tyrosine kinase inhibitor is crizotinib. In embodiments, the tyrosine kinase inhibitor is erlotinib. In embodiments, the tyrosine kinase inhibitor is gefitinib. In embodiments, the tyrosine kinase inhibitor is lapatinib. In embodiments, the tyrosine kinase inhibitor is pazopanib. In embodiments, the tyrosine kinase inhibitor is ruxolitinib. In embodiments, the tyrosine kinase inhibitor is sunitinib. In embodiments, the tyrosine kinase inhibitor is vemurafenib.

The disclosure provides methods of diagnosing myeloid leukemia in a patient, the method comprising: (i) measuring miR-142 levels in a biological sample obtained from the patient; and (ii) diagnosing the patient as having chronic myeloid leukemia when the miR-142 levels are reduced relative to a control. In embodiments, the methods further comprising administering to the patient diagnosed as having myeloid leukemia an effective amount of the compound of Formula (I) described herein, including any embodiment thereof. In embodiments, the methods comprise administering to the patient identified as having myeloid leukemia based on the reduced miR-142 levels an effective amount of a pharmaceutical composition comprising a pharmaceutically acceptable excipient and the compound of Formula (I) described herein, including any embodiment thereof. In embodiments, the myeloid leukemia is chronic myeloid leukemia. In embodiments, the chronic myeloid leukemia is the chronic phase of chronic myeloid leukemia. In embodiments, the chronic myeloid leukemia is the accelerated phase of chronic myeloid leukemia. In embodiments, the chronic myeloid leukemia is the blast phase of chronic myeloid leukemia. In embodiments, the myeloid leukemia is acute myeloid leukemia. In embodiments, the acute myeloid leukemia is secondary acute myeloid leukemia. In embodiments, the secondary acute myeloid leukemia is related to therapy. In embodiments, the secondary acute myeloid leukemia is related to an antecedent hematologic disorder. In embodiments, the antecedent hematologic disorder is myelodysplastic syndrome, myeloproliferative neoplasm, or myelodysplastic syndrome/myeloproliferative neoplasm overlap syndrome. In embodiments, the reduced miR-142 levels are in CD34⁺ cells, CD38⁻ cells, or both CD34⁺ cells and CD38⁻ cells. In embodiments, the myeloid leukemia is resistant to a tyrosine kinase inhibitor. In embodiments, the methods further comprising administering to the patient an effective amount of a tyrosine kinase inhibitor. In embodiments, the methods further comprising administering to the patient an effective amount of a tyrosine kinase inhibitor selected from the group consisting of imatinib, dasatinib, nilotinib, bosutinib, and ponatinib. In embodiments, the methods further comprising administering to the patient an effective amount of a tyrosine kinase inhibitor selected from the group consisting of imatinib, dasatinib, nilotinib, bosutinib, ponatinib, axitinib, crizotinib, erlotinib, gefitinib, lapatinib, pazopanib, ruxolitinib, sunitinib, and vemurafenib. In embodiments, the tyrosine kinase inhibitor is imatinib. In embodiments, the tyrosine kinase inhibitor is dasatinib. In embodiments, the tyrosine kinase inhibitor is nilotinib. In embodiments, the tyrosine kinase inhibitor is bosutinib. In embodiments, the tyrosine kinase inhibitor is ponatinib. In embodiments, the tyrosine kinase inhibitor is axitinib. In embodiments, the tyrosine kinase inhibitor is crizotinib. In embodiments, the tyrosine kinase inhibitor is erlotinib. In embodiments, the tyrosine kinase inhibitor is gefitinib. In embodiments, the tyrosine kinase inhibitor is lapatinib. In embodiments, the tyrosine kinase inhibitor is pazopanib. In embodiments, the tyrosine kinase inhibitor is ruxolitinib. In embodiments, the tyrosine kinase inhibitor is sunitinib. In embodiments, the tyrosine kinase inhibitor is vemurafenib.

The disclosure provides methods of treating aplastic anemia in a patient in need thereof by administering to the patient an effective amount of the compound of Formula (I) described herein, including any embodiment thereof. In embodiments, the methods comprise administering to the patient an effective amount of a pharmaceutical composition comprising a pharmaceutically acceptable excipient and the compound of Formula (I) described herein, including any embodiment thereof. In embodiments, a biological sample obtained from the patient has reduced miR-142 levels relative to a control. In embodiments, the reduced miR-142 levels are in CD34⁺ cells, CD38⁻ cells, or both CD34⁺ cells and CD38⁻ cells. In embodiments, the methods provide increased expression of miR-142. In embodiments, the methods provide increased expression of miR-142-3p and miR-142-5p. In embodiments, the methods provide reduced expression of the miR-142 target Baff-R in bone marrow cells. In embodiments, the aplastic anemia is resistant to a tyrosine kinase inhibitor. In embodiments, the methods further comprising administering to the patient an effective amount of a tyrosine kinase inhibitor. In embodiments, the methods further comprising administering to the patient an effective amount of a tyrosine kinase inhibitor selected from the group consisting of imatinib, dasatinib, nilotinib, bosutinib, and ponatinib. In embodiments, the methods further comprising administering to the patient an effective amount of a tyrosine kinase inhibitor selected from the group consisting of imatinib, dasatinib, nilotinib, bosutinib, ponatinib, axitinib, crizotinib, erlotinib, gefitinib, lapatinib, pazopanib, ruxolitinib, sunitinib, and vemurafenib. In embodiments, the tyrosine kinase inhibitor is imatinib. In embodiments, the tyrosine kinase inhibitor is dasatinib. In embodiments, the tyrosine kinase inhibitor is nilotinib. In embodiments, the tyrosine kinase inhibitor is bosutinib. In embodiments, the tyrosine kinase inhibitor is ponatinib. In embodiments, the tyrosine kinase inhibitor is axitinib. In embodiments, the tyrosine kinase inhibitor is crizotinib. In embodiments, the tyrosine kinase inhibitor is erlotinib. In embodiments, the tyrosine kinase inhibitor is gefitinib. In embodiments, the tyrosine kinase inhibitor is lapatinib. In embodiments, the tyrosine kinase inhibitor is pazopanib. In embodiments, the tyrosine kinase inhibitor is ruxolitinib. In embodiments, the tyrosine kinase inhibitor is sunitinib. In embodiments, the tyrosine kinase inhibitor is vemurafenib.

The disclosure provides method of preventing or delaying the progression of an antecedent clonal hematopoietic disorder in a patient in need thereof comprising administering to the patient an effective amount of the compound of Formula (I) described herein, including any embodiment thereof. In embodiments, the methods comprise preventing the progression of an antecedent clonal hematopoietic disorder. In embodiments, the methods comprise delaying the progression of an antecedent clonal hematopoietic disorder. In embodiments, the methods comprise preventing the progression of an antecedent clonal hematopoietic disorder to acute myeloid leukemia or blast crisis chronic myelogenous leukemia in a patient in need thereof comprising administering to the patient an effective amount of the compound of Formula (I) described herein, including any embodiment thereof. In embodiments, the methods comprise administering to the patient an effective amount of a pharmaceutical composition comprising a pharmaceutically acceptable excipient and the compound of Formula (I) described herein, including any embodiment thereof. In embodiments, the patient has the antecedent clonal hematopoietic disorder. In embodiments, the methods comprise preventing the progression of an antecedent clonal hematopoietic disorder to acute myeloid leukemia or blast crisis chronic myelogenous leukemia. In embodiments, the methods comprise delaying the progression of an antecedent clonal hematopoietic disorder to acute myeloid leukemia or blast crisis chronic myelogenous leukemia. In embodiments, the methods comprise preventing or delaying the progression of the antecedent clonal hematopoietic disorder to acute myeloid leukemia. In embodiments, the methods comprise preventing the progression of the antecedent clonal hematopoietic disorder to acute myeloid leukemia. In embodiments, the methods comprise delaying the progression of the antecedent clonal hematopoietic disorder to acute myeloid leukemia. In embodiments, the antecedent clonal hematopoietic disorder is an antecedent hematologic disorder. In embodiments, the antecedent hematologic disorder is myelodysplastic syndrome, myeloproliferative neoplasm, aplastic anemia, or myelodysplastic syndrome/myeloproliferative neoplasm overlap syndrome. In embodiments, the acute myeloid leukemia is secondary acute myeloid leukemia. In embodiments, the methods comprise preventing or delaying the progression of an antecedent clonal hematopoietic disorder to blast crisis chronic myelogenous leukemia. In embodiments, the methods comprise preventing the progression of an antecedent clonal hematopoietic disorder to blast crisis chronic myelogenous leukemia. In embodiments, the methods comprise delaying the progression of an antecedent clonal hematopoietic disorder to blast crisis chronic myelogenous leukemia. In embodiments, the antecedent clonal hematopoietic disorder is chronic phase chronic myelogenous leukemia. In embodiments, the antecedent clonal hematopoietic disorder is accelerated phase chronic myelogenous leukemia. In embodiments, a biological sample obtained from the patient has reduced miR-142 levels relative to a control. In embodiments, the reduced miR-142 levels are in CD34⁺ cells, CD38⁻ cells, or both CD34⁺ cells and CD38⁻ cells. In embodiments, the methods provide increased expression of miR-142. In embodiments, the methods provide increased expression of miR-142-3p and miR-142-5p. In embodiments, the methods provide reduced expression of the miR-142 target Baff-R in bone marrow cells. In embodiments, the aplastic anemia is resistant to a tyrosine kinase inhibitor. In embodiments, the methods further comprising administering to the patient an effective amount of a tyrosine kinase inhibitor. In embodiments, the methods further comprising administering to the patient an effective amount of a tyrosine kinase inhibitor selected from the group consisting of imatinib, dasatinib, nilotinib, bosutinib, and ponatinib. In embodiments, the methods further comprising administering to the patient an effective amount of a tyrosine kinase inhibitor selected from the group consisting of imatinib, dasatinib, nilotinib, bosutinib, ponatinib, axitinib, crizotinib, erlotinib, gefitinib, lapatinib, pazopanib, ruxolitinib, sunitinib, and vemurafenib. In embodiments, the tyrosine kinase inhibitor is imatinib. In embodiments, the tyrosine kinase inhibitor is dasatinib. In embodiments, the tyrosine kinase inhibitor is nilotinib. In embodiments, the tyrosine kinase inhibitor is bosutinib. In embodiments, the tyrosine kinase inhibitor is ponatinib. In embodiments, the tyrosine kinase inhibitor is axitinib. In embodiments, the tyrosine kinase inhibitor is crizotinib. In embodiments, the tyrosine kinase inhibitor is erlotinib. In embodiments, the tyrosine kinase inhibitor is gefitinib. In embodiments, the tyrosine kinase inhibitor is lapatinib. In embodiments, the tyrosine kinase inhibitor is pazopanib. In embodiments, the tyrosine kinase inhibitor is ruxolitinib. In embodiments, the tyrosine kinase inhibitor is sunitinib. In embodiments, the tyrosine kinase inhibitor is vemurafenib.

The term “delaying” with reference to delaying the progression of an antecedent clonal hematopoietic disorder means that the progression of the antecedent clonal hematopoietic disorder (e.g., progression to acute myeloid leukemia or blast crisis chronic myelogenous leukemia) occurs at a period of time that is longer than the progression would occur without administration of the compounds described herein. In embodiments, the delay in progression of an antecedent clonal hematopoietic disorder to acute myeloid leukemia or blast crisis chronic myelogenous leukemia can be weeks, months, or years.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In embodiments, chronic administration may be required. For example, the compositions are administered to the subject in an amount and for a duration sufficient to treat the patient. In embodiments, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, and other non-mammalian animals. In embodiments, a patient is human.

Cancer model organism, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g. mice) and from humans.

“Coadminister” means that compounds, nucleic acids, or pharmaceutical composition described herein is administered at the same time, just prior to, or just after the administration of one or more additional tyrosine kinase inhibitors, anti-inflammatory agents, anti-cancer agents and/or radiation treatment. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation).

The singular terms “a”, “an”, and “the” include the plural reference unless the context clearly indicates otherwise.

The term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

### Informal Sequence Listing

In the sequence listings, * is phosphorothioation; a double quote after the nucleic acid (e.g., U″) means the nucleic acid is modified with 2′-O-methyl, and a single quote after the nucleic acid (e.g., U′) means that the nucleic acid is modified with 2′-fluoro; and U^{p} means that the nucleic acid has a single phosphate group.

SEQ ID NO: 1 = miR142 passenger strand sequence:
5′ CAU AAA GUA GGA AAC ACU ACA AA 3′
SEQ ID NO: 2 = miR142 passenger strand sequence:
5′ UCCAUAAAGUAGGAAACACUACA 3′
SEQ ID NO: 3 (miR142 mimic passenger strand):
5′ C”*A”*U”A”A”A”G’U”A’G′G’A”A”A”C”A”C”U”A”C”A” 3′
SEQ ID NO: 4 = miR142 guide strand sequence:
5′ UGUAGUGUUUCCUACUUUAUGGA3′
SEQ ID NO: 5 (miR142 mimic guide strand):
5′ U”*G’*U”A”G”U’G”U”U”U”C”C”U”A’C”U′U”U”A”U”G”*
G”*A” 3′
SEQ ID NO: 26 = miR142 passenger strand sequence:
5′ CAU AAA GUA GGA AAC ACU ACA A”A
SEQ ID NO: 27 = miR142 passenger strand sequence:
5′ UAG UGC UUU CUA CUU UAU GA”A
SEQ ID NO: 28 = miR142 guide strand sequence:
5′ CAU AAA GUA GAA AGC ACU ACU

### EMBODIMENTS

Embodiment 1. A compound of Formula (I): R¹-L¹-R² (I); wherein: R¹ is a CpG oligodeoxynucleotide (ODN); L¹ is a linking group; R² is a hybridized nucleic acid sequence comprising: (i) a miR-142 passenger strand sequence comprising SEQ ID NO:1 hybridized to a miR-142 guide strand sequence comprising SEQ ID NO:4; (ii) a miR-142 passenger strand sequence comprising SEQ ID NO:2 hybridized to a miR-142 guide strand sequence comprising SEQ ID NO:4; or (iii) a miR-142 passenger strand sequence comprising SEQ ID NO:3 hybridized to a miR-142 guide strand sequence comprising SEQ ID NO:5.

Embodiment 2. A compound of Formula (I): R¹-L¹-R² (I); wherein: R¹ is a CpG oligodeoxynucleotide (ODN); L¹ is a linking group; R² is a hybridized nucleic acid sequence comprising: (i) a miR-142 passenger strand sequence comprising SEQ ID NO:1 hybridized to a miR-142 guide strand sequence comprising SEQ ID NO:4; (ii) a miR-142 passenger strand sequence comprising SEQ ID NO:2 hybridized to a miR-142 guide strand sequence comprising SEQ ID NO:4; (iii) a miR-142 passenger strand sequence comprising SEQ ID NO:3 hybridized to a miR-142 guide strand sequence comprising SEQ ID NO:5; (iv) a miR-142 passenger strand sequence comprising SEQ ID NO:26 hybridized to a miR-142 guide strand sequence comprising SEQ ID NO:4; or (v) a miR-142 passenger strand sequence comprising SEQ ID NO:27 hybridized to a miR-142 guide strand sequence comprising SEQ ID NO:28.

Embodiment 3. The compound of Embodiment 1 or 2, wherein R² is the hybridized nucleic acid sequence which comprises the miR-142 passenger strand sequence comprising SEQ ID NO:1 hybridized to the miR-142 guide strand sequence comprising SEQ ID NO:4.

Embodiment 4. The compound of Embodiment 1 or 2, wherein R² is the hybridized nucleic acid sequence which comprises the miR-142 passenger strand sequence comprising SEQ ID NO:2 hybridized to the miR-142 guide strand sequence comprising SEQ ID NO:4.

Embodiment 5. The compound of Embodiment 2, wherein R² is the hybridized nucleic acid sequence which comprises the miR-142 passenger strand sequence comprising SEQ ID NO:26 hybridized to the miR-142 guide strand sequence comprising SEQ ID NO: 4.

Embodiment 6. The compound of Embodiment 2, wherein R² is the hybridized nucleic acid sequence which comprises the miR-142 passenger strand sequence comprising SEQ ID NO:27 hybridized to the miR-142 guide strand sequence comprising SEQ ID NO:28.

Embodiment 7. The compound of any one of Embodiments 1 to 6, wherein one or more nucleotides in the miR-142 passenger strand sequence are modified with 2′-O-Methyl, 2′-Fluoro, a phosphorothioate moiety, or a combination thereof.

Embodiment 8. The compound of any one of Embodiments 1 to 7, wherein one or more nucleotides in the miR-142 guide strand sequence are modified with 2′-O-Methyl, 2′-Fluoro, a phosphorothioate moiety, or a combination thereof.

Embodiment 9. The compound of Embodiment 1 or 2, wherein R² is a hybridized nucleic acid sequence which comprises the miR-142 passenger strand sequence comprising SEQ ID NO:3 hybridized to the miR-142 guide strand sequence comprising SEQ ID NO:5.

Embodiment 10. The compound of any one of Embodiments 1 to 9, wherein the 3′ end of R¹ is covalently bonded to L¹.

Embodiment 11. The compound of any one of Embodiments 1 to 10, wherein the miR-142 passenger strand sequence is covalently bonded to the linking group; and wherein the miR-142 guide strand sequence is hybridized to the miR-142 passenger strand sequence.

Embodiment 12. The compound of any one of Embodiments 1 to 11, wherein R¹ comprises SEQ ID NO:8.

Embodiment 13. The compound of any one of Embodiments 1 to 11, wherein R¹ comprises SEQ ID NO:6.

Embodiment 14. The compound of any one of Embodiments 1 to 11, wherein the R¹ comprises a CpG-A ODN, a CpG-B ODN, or a CpG-C ODN.

Embodiment 15. The compound of any one of Embodiments 1 to 11, wherein R¹ comprises CpG ODN 19, CpG ODN 1585, CpG ODN 2216, CpG ODN 2336, CpG ODN 1668, CpG ODN 1826, CpG ODN 2006, CpG ODN 2007, CpG ODN BW006, CpG ODN D-SL01, CpG ODN 2395, CpG ODN M362, or CpG ODN D-SL03.

Embodiment 16. The compound of any one of Embodiments 1 to 11, wherein R¹ comprises the sequence of any one of SEQ ID NOS:7-25.

Embodiment 17. The compound of any one of Embodiments 1 to 16, wherein one or more nucleotides in the CpG oligodeoxynucleotide (ODN) of R¹ are modified with a phosphorothioate moiety.

Embodiment 18. The compound of any one of Embodiments 1 to 17, wherein L¹ is a bond, a nucleic acid sequence, substituted or unsubstituted alkylene, a substituted or unsubstituted heteroalkylene, or a combination of two or more thereof.

Embodiment 19. The compound of Embodiment 18, wherein L¹ is a substituted 6 to 48 membered heteroalkylene; wherein the heteroalkylene comprises an oxygen atom, a phosphorous atom, or a combination thereof; and wherein the substituents are independently selected from the group consisting of ═O, —OH, and —O⁻.

Embodiment 20. The compound of Embodiment 19, wherein L¹ is:

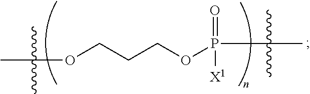

wherein X¹ is independently —OH or —O⁻, and n is an integer from 1 to 8.

Embodiment 21. The compound of Embodiment 20, wherein n is an integer from 4 to 6.

Embodiment 22. The compound of Embodiment 21, wherein n is 5.

Embodiment 23. The compound of any one of Embodiments 1 to 22, wherein the 5′ end of the miR-142 passenger strand sequence is covalently bonded to L¹.

Embodiment 24. A pharmaceutical composition comprising the compound of any one of Embodiment 1 to 23 and a pharmaceutically acceptable excipient.

Embodiment 25. A method of treating myeloid leukemia in a patient in need thereof, the method comprising administering to the patient an effective amount of the compound of any one of Embodiments 1 to 23 or the pharmaceutical composition of Embodiment 24.

Embodiment 26. The method of Embodiment 25, wherein a biological sample obtained from the patient has reduced miR-142 levels relative to a control.

Embodiment 27. A method of treating myeloid leukemia in a patient in need thereof, the method comprising: (i) detecting reduced miR-142 levels relative to a control in a biological sample obtained from the patient; (ii) administering to the patient an effective amount of the compound of any one of Embodiments 1 to 23 or the pharmaceutical composition of Embodiment 24.

Embodiment 28. A method of treating myeloid leukemia in a patient in need thereof, the method comprising: (i) measuring miR-142 levels in a biological sample obtained from the patient; (ii) identifying the patient as having myeloid leukemia when the miR-142 levels are reduced relative to a control; and (iii) administering to the patient identified as having myeloid leukemia based on the reduced miR-142 levels an effective amount of the compound of any one of Embodiments 1 to 23 or the pharmaceutical composition of Embodiment 24.

Embodiment 29. A method of diagnosing myeloid leukemia in a patient, the method comprising: (i) measuring miR-142 levels in a biological sample obtained from the patient; and (ii) diagnosing the patient as having myeloid leukemia when the miR-142 levels are reduced relative to a control.

Embodiment 30. The method of Embodiment 29, further comprising administering to the patient diagnosed as having myeloid leukemia an effective amount of the compound of any one of Embodiments 1 to 23 or the pharmaceutical composition of Embodiment 24.

Embodiment 31. The method of any one of Embodiments 25 to 30, wherein the myeloid leukemia is chronic myeloid leukemia.

Embodiment 32. The method of Embodiment 31, wherein the chronic myeloid leukemia is chronic phase.

Embodiment 33. The method of Embodiment 31, wherein the chronic myeloid leukemia is accelerated phase.

Embodiment 34. The method of Embodiment 31, wherein the chronic myeloid leukemia is blast crisis phase.

Embodiment 35. The method of any one of Embodiments 25 to 30, wherein the myeloid leukemia is acute myeloid leukemia.

Embodiment 36. The method of Embodiment 35, wherein the acute myeloid leukemia is secondary acute myeloid leukemia.

Embodiment 37. The method of Embodiment 36, wherein the secondary acute myeloid leukemia is related to therapy.

Embodiment 38. The method of Embodiment 36, wherein the secondary acute myeloid leukemia is related to an antecedent hematologic disorder.

Embodiment 39. The method of Embodiment 38, wherein the antecedent hematologic disorder is myelodysplastic syndrome, myeloproliferative neoplasm, or myelodysplastic syndrome/myeloproliferative neoplasm overlap syndrome.

Embodiment 40. The method of any one of Embodiments 26 to 39, wherein the reduced miR-142 levels are in reduced in CD34⁺ cells, reduced in CD38⁻ cells, or reduced in CD34⁺ cells and CD38⁻ cells.

Embodiment 41. The method of any one of Embodiments 25 to 40, wherein the myeloid leukemia is resistant to a tyrosine kinase inhibitor.

Embodiment 42. The method of any one of Embodiments 25 to 41, further comprising administering to the patient an effective amount of a tyrosine kinase inhibitor.

Embodiment 43. The method of Embodiment 41 or 42, wherein the tyrosine kinase inhibitor is imatinib, dasatinib, nilotinib, bosutinib, or ponatinib.

Embodiment 44. The method of Embodiment 41 or 42, wherein the tyrosine kinase inhibitor imatinib, dasatinib, nilotinib, bosutinib, ponatinib, axitinib, crizotinib, erlotinib, gefitinib, lapatinib, pazopanib, ruxolitinib, sunitinib, and vemurafenib.

Embodiment 45. A method of treating myelodysplastic syndrome, myeloproliferative neoplasm, or myelodysplastic syndrome/myeloproliferative neoplasm overlap syndrome in a patient in need thereof, the method comprising administering to the patient an effective amount of the compound of any one of Embodiments 1 to 23 or the pharmaceutical composition of Embodiment 24.

Embodiment 46. The method of Embodiment 45, wherein a biological sample obtained from the patient has reduced miR-142 levels relative to a control.

Embodiment 47. A method of treating myelodysplastic syndrome, myeloproliferative neoplasm, or myelodysplastic syndrome/myeloproliferative neoplasm overlap syndrome in a patient in need thereof, the method comprising: (i) detecting reduced miR-142 levels relative to a control in a biological sample obtained from the patient; and (ii) administering to the patient an effective amount of the compound of any one of Embodiments 1 to 23 or the pharmaceutical composition of Embodiment 24.

Embodiment 48. A method of treating myelodysplastic syndrome, myeloproliferative neoplasm, or myelodysplastic syndrome/myeloproliferative neoplasm overlap syndrome in a patient in need thereof, the method comprising: (i) measuring miR-142 levels in a biological sample obtained from the patient; (ii) identifying the patient as having myelodysplastic syndrome, myeloproliferative neoplasm, or myelodysplastic syndrome/myeloproliferative neoplasm overlap syndrome when the miR-142 levels are reduced relative to a control; and (iii) administering to the patient identified as having myelodysplastic syndrome, myeloproliferative neoplasm, or myelodysplastic syndrome/myeloproliferative neoplasm overlap syndrome based on the reduced miR-142 levels an effective amount of the compound of any one of Embodiments 1 to 23 or the pharmaceutical composition of Embodiment 24.

Embodiment 49. The method of any one of Embodiments 45 to 48, comprising treating myelodysplastic syndrome in the patient.

Embodiment 50. The method of any one of Embodiments 45 to 48, comprising treating myeloproliferative neoplasm in the patient.

Embodiment 51. The method of any one of Embodiments 45 to 48, comprising treating myelodysplastic syndrome/myeloproliferative neoplasm overlap syndrome in the patient.

Embodiment 52. A method of treating aplastic anemia in a patient in need thereof, the method comprising administering to the patient an effective amount of the compound of any one of Embodiments 1 to 23 or the pharmaceutical composition of Embodiment 24.

Embodiment 53. The method of Embodiment 52, wherein a biological sample obtained from the patient has reduced miR-142 levels relative to a control.

Embodiment 54. A method of treating aplastic anemia in a patient in need thereof, the method comprising: (i) detecting reduced miR-142 levels relative to a control in a biological sample obtained from the patient; and (ii) administering to the patient an effective amount of the compound of any one of Embodiments 1 to 23 or the pharmaceutical composition of Embodiment 24.

Embodiment 55. A method of treating aplastic anemia in a patient in need thereof, the method comprising: (i) measuring miR-142 levels in a biological sample obtained from the patient; (ii) identifying the patient as having aplastic anemia when the miR-142 levels are reduced relative to a control; and (iii) administering to the patient identified as having aplastic anemia based on the reduced miR-142 levels an effective amount of the compound of any one of Embodiments 1 to 23 or the pharmaceutical composition of Embodiment 24.

Embodiment 56. A method of preventing or delaying the progression of an antecedent clonal hematopoietic disorder in a patient in need thereof, the method comprising administering to the patient an effective amount of the compound of any one of Embodiments 1 to 23 or the pharmaceutical composition of Embodiment 24; wherein the patient has the antecedent clonal hematopoietic disorder.

Embodiment 57. A method of preventing or delaying the progression of an antecedent clonal hematopoietic disorder to acute myeloid leukemia or blast crisis chronic myelogenous leukemia in a patient in need thereof, the method comprising administering to the patient an effective amount of the compound of any one of Embodiments 1 to 23 or the pharmaceutical composition of Embodiment 24; wherein the patient has the antecedent clonal hematopoietic disorder.

Embodiment 58. The method of Embodiment 56 or 57 for preventing the progression of the antecedent clonal hematopoietic disorder to acute myeloid leukemia.

Embodiment 59. The method of Embodiment 56 or 57 for delaying the progression of the antecedent clonal hematopoietic disorder to acute myeloid leukemia.

Embodiment 60. The method of Embodiment 58 or 59, wherein the antecedent clonal hematopoietic disorder is an antecedent hematologic disorder.

Embodiment 61. The method of Embodiment 60, wherein the antecedent hematologic disorder is myelodysplastic syndrome, myeloproliferative neoplasm, aplastic anemia, or myelodysplastic syndrome/myeloproliferative neoplasm overlap syndrome.

Embodiment 62. The method of any one of Embodiments 57 to 60, wherein the acute myeloid leukemia is secondary acute myeloid leukemia.

Embodiment 63. The method of Embodiment 56 or 57 for preventing the progression of an antecedent clonal hematopoietic disorder to blast crisis chronic myelogenous leukemia.

Embodiment 64. The method of Embodiment 56 or 57 for delaying the progression of an antecedent clonal hematopoietic disorder to blast crisis chronic myelogenous leukemia.

Embodiment 65. The method of Embodiment 63 or 64, wherein the antecedent clonal hematopoietic disorder is chronic phase chronic myelogenous leukemia.

Embodiment 66. The method of Embodiment 65, wherein the antecedent clonal hematopoietic disorder is accelerated phase chronic myelogenous leukemia.

Embodiment 67. The method of any one of Embodiments 52 to 66, further comprising administering to the patient an effective amount of a tyrosine kinase inhibitor.

Embodiment 68. The method of Embodiment 67, wherein the tyrosine kinase inhibitor is imatinib, dasatinib, nilotinib, bosutinib, ponatinib, axitinib, crizotinib, erlotinib, gefitinib, lapatinib, pazopanib, ruxolitinib, sunitinib, or vemurafenib.

Embodiment 69. The method of Embodiment 67, wherein the tyrosine kinase inhibitor is imatinib, dasatinib, nilotinib, bosutinib, or ponatinib.

### EXAMPLES

It is understood that the examples described herein are for illustrative purposes only and that various modifications or changes in light thereof will be suggested to persons skilled in the art and are to be included within the spirit and purview of this application and scope of the appended claims.

MicroRNAs (miRNAs) are small non-coding RNAs of 18-24 nucleotides that hybridize to target messenger RNAs (mRNAs) causing their translation inhibition and/or degradation. Deregulation of miRNAs has been associated with a variety of cancers and leukemia, including AML. miR142 located at 17q22 and encoding miR-142 is a highly conserved “gene” that is highly expressed in hematopoietic cells. Two mature miRNAs (miR-142-3p and miR-142-5p) are derived from opposite strands of the encoded miR-142 hairpin-like precursor (pre-miR-142). Knockdown (KD) of miR-142 expression in the zebrafish and the mouse revealed a critical role for this miRNA in hematopoiesis. In the mouse, miR142 loss associates with expansion of BM HPSCs, decreased hematopoietic output, splenomegaly and reduction of peripheral T and B cells and platelets. These changes are accompanied by profound immunodeficiency.

miR142 was the only miRNA originally reported to be mutated in an AML TCGA study, and our own data showed that miR-142-3p levels were significantly reduced in BM mononuclear cells (MNCs) from AML patients compared with healthy donors. Furthermore, miR-142 is downregulated in BC CML patients compared with CP CML patients. More recently, we demonstrated that miR-142 KO in mouse models of CP CML or FLT3-ITD MPN prompts transformation of the respective myeloproliferative phenotypes into an AML- and myeloid BC-like diseases with significantly shorter survivals. Our data support a role of miR-142 deficit in the deregulation of the clonal hematopoietic stem cells' (HSCs) metabolism, with a switch to higher levels of oxidative phosphorylation (OxPhosp) via increased fatty acid oxidation (FAO). We therefore hypothesized that these changes may play an important role in the evolution of clonal HSCs to sAML/BC CML leukemic stem cells (LSCs) and in turn for development of the AML- and BC-like phenotypes. Of note, in vitro and in vivo rescue of miR-142 deficit with a novel miR-142 mimic compound (named CpG-M-miR-142, FIG. 15A) that we designed and manufactured, led to a decrease in OxPhos, cell viability, and burden of sAML/BC CML LSCs. Thus, we describe herein a cellular and molecular basis of miR-142 downregulation and its impact on the transformation of clonal hematopoietic disorders into a more aggressive AML- and BC-like diseases, for which we designed novel treatments to replace the otherwise low levels of miR-142 and produce a significant clinical benefit for patients with sAML and BC CML.

Chronic myeloid leukemia (CML) is a myeloproliferative neoplasm associated with the Philadelphia chromosome t(9;22)(q34;q11) resulting in the BCR-ABL1 fusion gene that encodes a constitutively activated tyrosine kinase (TK). Although TK inhibitors (TKIs) are effective in inducing disease remission and prolonged survival in CML patients, a subset of them are either intolerant or resistant and ultimately progress from chronic phase (CP) to blast crisis (BC), which responds poorly to TKI treatment and carries poor prognosis. Therefore, understanding the molecular mechanisms through which CML transforms from CP to BC is a necessary step for developing novel strategies that effectively prevent or treat transformation.

Acute myeloid leukemia (AML) is a hematopoietic malignancy characterized by acquired mutations and aberrant expression of genes involved in normal hematopoiesis. (Ref 1). These genetic abnormalities cause cell differentiation arrest and accumulation of hematopoietic stem and progenitor cells (HSPCs) and partially differentiated “bulk” blasts that lead to bone marrow (BM) failure. Despite improvements in our understanding of the pathogenesis of this disease and emerging novel molecular targeted therapeutics, the overall outcome of these patients remains poor. In 2019, more than 21,000 new AML cases have been diagnosed in the US (median age 72 years) and more than 10,000 AML patients have died of their disease. While an increasing number of AML patients have an available donor and undergo allogeneic hematopoietic stem cell transplantation (alloHSCT) for a better chance of a cure, the inherent transplant-related morbidity and mortality offset the clinical benefit of this approach. Thus, with an estimated cumulative 5-year survival at a disappointing rate of about 28% (2010-2016), much needs to be done to improve the outcome of these individuals. (Ref 2).

Prognostic assessment and risk-stratification therapies are the backbone of the current clinical approach to AML patients and are mainly based on cytogenetic and molecular findings. (Ref 3). However, consideration of additional clinical features including age, co-morbidities, prior radiation or chemotherapy, and antecedent clonal hematopoietic disorders (ACHD) (i.e., myeloproliferative neoplasm (MPN; Primary Myelofibrosis, Essential Thrombocythemia, Polycythemia Vera), myelodysplastic syndrome (MDS), MDS/MPN; (i.e., chronic myelomonocytic leukemia (CMML)), is key for proper selection of effective therapeutic programs. (Ref 4, 5). Of note, AML patients with therapy-related disease (t-AML), and those with history of ACDH (secondary (s) AML) have a significantly worse prognosis than patients with de novo AML and are often excluded from promising clinical trials. (Ref 6). Furthermore, while BCR-ABL+ AML is a recognized entity in the World Health Organization classification of myeloid neoplasms, the majority of BCR-ABL+ patients with AML-like disease have a previous history of chronic phase (CP) chronic myelogenous leukemia (CML) that has progressed to myeloid blast crisis (BC), and they also have a poor prognosis. (Ref 7, 8). Thus, the availability of novel and more effective treatments is a true unmet need for t-AML, sAML and BC CML patients. In this proposal, we will focus on sAML and BC CML.

MicroRNA-142 (miR-142) in normal and clonal hematopoiesis. MicroRNAs (miRNAs) are small non-coding RNAs of 18-24 nucleotides that hybridize to target messenger RNAs (mRNAs) causing their translation inhibition and/or degradation. Deregulation of miRNAs has been associated with a variety of cancers and leukemia, including AML. (Ref 9).

MIR142 located at 17q22 and encoding miR-142 is a highly conserved “gene”, highly expressed in hematopoietic cells. Two mature miRNAs (miR-142-3p and miR-142-5p) are derived from opposite strands of the encoded miR-142 hairpin-like precursor (pre-miR-142). Knockdown (KD) of miR-142-3p expression in zebrafish revealed a critical role for this miRNA in hematopoiesis with reduced activity of HSPCs. In the mouse, Mir142 is also highly expressed in hematopoietic cells and is involved in the development and function of B and T lymphocytes, myeloid and natural killer (NK) cells, and megakaryocyte-erythroid precursors. (Refs 10-18).

In the mouse, Mir142 loss associates with expansion of BM HPSCs, decreased hematopoietic output, splenomegaly and reduction of peripheral T and B cells and platelets. These changes are accompanied by a profound immunodeficiency, manifested as hypoimmunoglobulinemia and failure to mount an efficient immune response to infections. Of note, MIR142 was found mutated in follicular and diffuse large B cell lymphoma and significantly downregulated in acute lymphocytic leukemia (ALL). (Ref 12, 19).

MIR142 was also reported to be mutated and/or downregulated in AML. Our own data also showed that miR-142-3p levels were significantly reduced in BM mononuclear cells (MNCs) from AML patients compared with healthy donors. Furthermore, miR-142 is downregulated in BC CML patients compared with CP CML patients. Mechanistically, Mir142 loss reportedly increased the leukemia initiating capacity of clonal hematopoietic stem cells (HSCs) harboring IDH2 mutations, partly by increasing the expression of the target gene Ash11 (absent, small, or homeotic 1-like), a histone methyltransferase that upregulates Hoxa9/10 expression. (Ref 20, 21).

More recently, we crossed a miR-142 KO mouse with models of clonal myeloproliferation disorders (MPD*) which do not transform spontaneously to AML. We discovered that abrogation of miR-142 expression is a single event causing phenotypic transformation of the clonal myeloproliferation of FLT3-ITD+ and BCR-ABL+ mice into sAML and BC CML, respectively. Thus, given a lack of effective therapies for patients with sAML and BC CML, herein we propose to dissect the molecular mechanisms through which miR-142 deficit contributes to the transformation of MPD** into sAML/BC CML and test the principle that restoring miR-142 expression is an effective therapeutic approach for the prevention and treatment of these conditions. To this end, we have developed a strategy that will pave the way for rapid translation into the clinic of a novel CpG mimic (M) miR-142, hereafter referred to as CpG-M-miR-142 (FIG. 15A).

We will use the term MPD to indicate comprehensively MPN and CML (for human) and if referred to in murine models, this term also includes the FLT3-ITD+ mouse, which presents with clonal myeloproliferation but not overt AML. Furthermore, to indicate self-renewal stem cells in different models, we will use the term normal HSCs for normal hematopoiesis, clonal HSCs for MPD, and leukemia stem cells (LSCs) for sAML and BC CML. (Ref 22).

The role of miR-142 in inducing progression from clonal MPD to AML has not been elucidated. We have postulated and demonstrated that deficiency of miR-142 transforms clonal BCR-ABL+ and FLT3^{ITD/ITD}+ HSCs into LSCs that generate, respectively, BC CML and sAML phenotypes. We now propose to test the novel hypothesis that miR-142 deficit is a “single” event able to induce transformation of clonal hematologic disorders into sAML or BC CML using novel murine models and patient-derived xenografts (PDXs).

To this end, we first noted that restoring miR-142 levels had significant antileukemic activity, and reduced the LSC burden in the BC CML mouse. Thus, we have now designed and manufactured a novel compound, CpG-M-miR-142 (FIG. 15), to rescue the miR-142 deficit and in turn prevent or treat sAML and BC CML.

We have created novel murine models of sAML and BC CML by crossing mice with MPD (FLT3-ITD+ or BCR-ABL+) with the global miR-142 KO mouse. We will expand the catalog of these strains using additional MPD and MPD/MDS models (i.e., JAK2^{V617F} and TET2null mice). Furthermore, using Cre-Lox recombination approaches, we will knock out (KO) miR-142 in distinct hematopoietic and non-hematopoietic cell subpopulations (compartments) of the BM niche, to study the role of compartmental miR-142 deficit in the BM niche during sAML or BC CML transformation.

### Example 1

miR-142 deficit promotes blast crisis (BC) transformation of chronic myelogenous leukemia (CML) mice. miR-142 deficit also promotes transformation of FLT3^{ITD/ITD} myeloproliferative neoplasm (MPN) to acute myeloid leukemia (AML). miR-142 deficit promotes TKI resistance in BCR-ABL+ cells. Therefore, we designed CpG-M-miR-142 shown in FIG. 15A. To obtain evidence of activity of CpG-M-miR-142 in vivo, we engrafted congenic recipient mice (CD45.1) with BM cells from miR-142 KO BC CML mice (CD45.2) and treated them with CpG-M-miR-142 (20 mg/kg/day, IV) or SCR for 3 w. Upon completion of treatment, we observed reduced blasts in PB and BM and smaller spleens in CpG-M-miR-142-treated mice compared with SCR-treated controls. We also detected a significantly increased expression of both miR-142-3p- and -5p and reduced expression of the miR-142 target Baff-R in BM cells from CpG-M-miR-142-treated mice vs SCR-treated mice. Another cohort of miR-142 KO BC CML mice were divided into two groups and treated with CpG-M-miR-142 (20 mg/kg/day, IV) or SCR for 4 weeks. The CpG-M-miR-142-treated mice survived significantly longer than SCR-treated mice (median survival: vs days, p=). Secondary recipients transplanted with BM cells from CpG-M-miR-142-treated donors showed reduced blood engraftment (i.e., disease burden) at 8 weeks post transplantation and significantly increased survival (median survival: vs days, p=) compared with recipients transplanted with BM cells from SCR-treated donors, indicating that CpG-M-miR-142 decreased LSC burden.

### Example 2

Here, we used the inducible SCLtTA/BCR-ABL transgenic mouse in a B6 background, a well characterized CP CML model, to study the molecular mechanism of disease evolution. Upon tetracycline withdrawal to induce BCR-ABL expression, both the SCLtTA/BCR-ABL homozygous (homo, i.e., SCLtTA^{+/+}BCR-ABL^{+/+}, hereafter called BCR-ABL) and heterozygous (het, i.e., SCLtTA^{+/-}BCR-ABL^{+/-}) transgenic mice developed and died of CP CML without developing BC CML, thereby implying that BCR-ABL dosage is insufficient to induce transformation in this mouse.

Among microRNAs (miR), miR-142 is highly expressed in hematopoietic cells and plays a critical role in normal hematopoiesis. In a miR-142 knockout (KO)(miR-142^{-/-}) mouse, we noted expansion of hematopoietic stem and progenitor cells and decrease of hematopoietic output. Aberrant miR-142 downregulation or mutations have been reported in other hematologic malignancies including lymphoma, acute lymphocytic leukemia and acute myeloid leukemia. Of note, we have also observed lower levels of miR-142 in CD34⁺ and CD34⁺CD38⁻ cells from patients with BC CML than in those from patients with CP CML. Thus, based on these data, we hypothesized that miR-142 insufficiency could play a role in the transformation of CP CML to BC.

To test our hypothesis, we generated miR-142 KO BCR-ABL transgenic (i.e., miR-142^{-/-}BCR-ABL) mice by crossing miR-142^{-/-} with BCR-ABL mice. We observed increasing circulating leukemic blasts over time after BCR-ABL induction in miR-142^{-/-}BCR-ABL mice, but not in miR-142 wt (miR-142^{+/+}) BCR-ABL controls even when the latter became moribund. MiR-142^{-/-}BCR-ABL mice also had larger spleens and significantly shorter survival (median survival: 26 vs 54 days; p<0.0001) than the miR-142^{+/+}BCR-ABL controls. Of note, while both homo (miR-142^{-/-}) and het (miR-142^{+/-}) miR-142 KO BCR-ABL mice eventually developed BC CML, the former had a significantly more rapid progression to BC and a shorter survival (median survival: 26 vs 45 days; p=0.003) than the latter. These results suggested that miR-142 deficiency, as a single event, is sufficient to initiate BC transformation in the CP CML model in a dose-dependent manner. Importantly, all these features were recapitulated in congenic recipient mice (CD45.1) transplanted with a relatively small number of BM LSKs (2000 cells/mouse) from diseased miR-142^{-/-}BCR-ABL mice (CD45.2), supporting that this cell subpopulation was enriched in miR-142^{-/-}BCR-ABL leukemic stem cells (LSCs) able to produce BC. Of note, in an RNA-seq analysis comparing LSKs from diseased miR-142 BCR-ABL (BC) and miR-142^{+/+} BCR-ABL(CP) mice, we found 504 genes to be differentially expressed. Gene set enrichment analysis (GSEA) showed that only four pathways were differentially expressed (upregulated); three of them [i.e., oxidative phosphorylation (OxPhos), glycolysis and adipogenesis] regulate cell metabolism and the fourth one regulates protein secretion.

To test if restoring miR-142 is a feasible treatment approach for BC, we developed a novel CpG-miR-142 mimic oligonucleotide (ODN), hereafter referred to as CpG-M-miR-142 shown in FIG. 15A. Treatment with CpG-M-miR-142 (20 mg/kg/day, iv, 4 weeks) started at the second day after BCR-ABL induction significantly prolonged the survival of miR-142^{-/-}BCR-ABL mice compared with treatment with CpG-scramble (SCR) (75% vs 33% survival rate at 40 days after BCR-ABL induction; median survival: not reached vs 25 days; p=0.03). Since we also observed that miR-142 levels were significantly lower in CD34⁺ cells from patients with BC CML and from TKI-resistant patients with CP CML than in CD34⁺ cells from TKI-sensitive patients with CP CML (p=0.02), to evaluate if downregulation of miR-142 also associates with TKI resistance, we selected LSKs from diseased miR-142^{-/-}BCR-ABL and miR-142^{+/+}BCR-ABL mice and exposed them to the TKI nilotinib (NIL; 2 μM) or vehicle for 72 hours. We observed lower apoptosis and higher cell growth in NIL-treated miR-142^{-/-}BCR-ABL LSKs than in NIL-treated miR-142^{+/+}BCR-ABL LSKs. The decreased sensitivity of miR-142^{-/-}BCR-ABL LSKs to TKI was rescued by treatment with CpG-M-miR-142. CpG-M-miR-142 (2 μM) plus NIL significantly increased apoptosis and reduced cell growth in miR-142^{-/-}BCR-ABL LSKs as compared with SCR+NIL.

In conclusion, we showed a key role of miR-142 deficiency in the transformation of CP CML to BC CML, which associated preferentially with deregulation of metabolic pathways. Restoring miR-142 expression in vivo with CpG-M-miR-142 treatment significantly decreased the BC transformation rate, prolonged survival of miR-142^{-/-}BCR-ABL mice and increased sensitivity to TKIs.

### Example 3

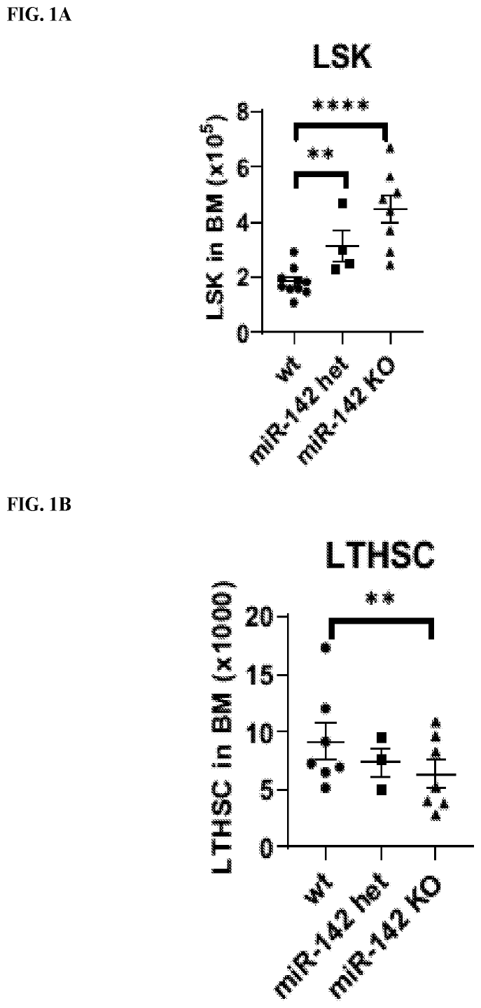

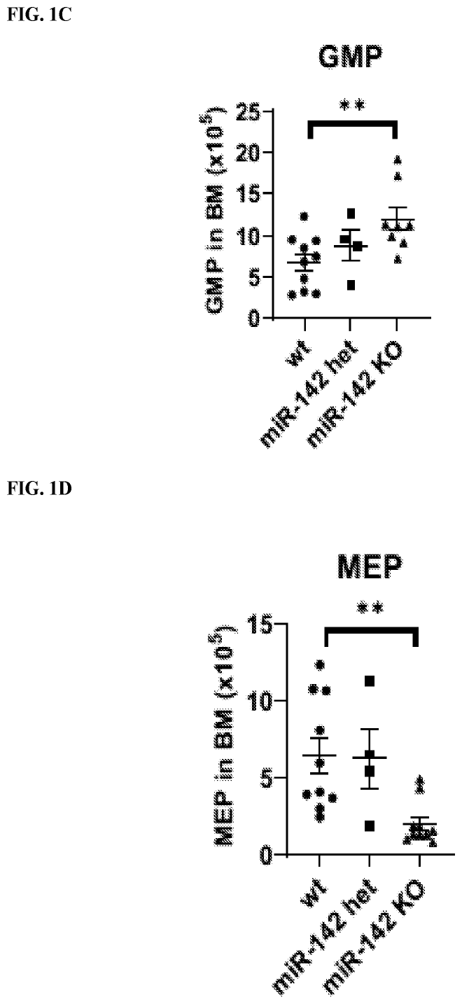

The miR-142^{-/-} mouse. To study the role of miR-142 in normal hematopoiesis and leukemogenesis, we imported a mouse line with a targeted deletion of the Mir142 gene. The miR-142^{-/-} mice displayed virtually no expression of either mature miR-142-3p or miR-142-5p in BM cells and appeared healthy and fertile. Gross morphologic analysis at necropsy revealed no organ defects, except for splenomegaly. In peripheral blood (PB), we observed a reduced number of white blood cell (WBC) counts [i.e., T (CD3⁺), B (CD19⁺) and mature myeloid (CD11b⁺/Gr-1⁺) cells], erythrocytes and platelets (PLT), and a few circulating multipotent hematopoietic cells (Lin⁻Sca-1⁺c-Kit⁺; LSK) and myeloid progenitors (Lin⁻Sca-1⁻c-Kit⁺; L⁻S⁻K⁺). In BM, we observed an increased number of LSKs, common lymphoid progenitors (CLPs, Lin⁻Sca-1^{Low}c-Kit^{Low}Flt3^{Hi}_{g}_{^{h}}IL-7_{α}_{^{Hi}}_{g}_{^{h}}), granulocyte-macrophage progenitors (GMPs, Lin⁻Sca1⁻cKit⁺CD34⁺FcγRII/II^{hi}) and common myeloid progenitors (CMPs, Lin⁻Sca1⁻cKit⁺CD34⁺FcγRII/II^{low}), and a reduced number of megakaryocyte-erythrocyte progenitors (MEPs, Lin⁻Sca1⁻cKit⁺CD34⁺FcγRII/II⁻) (FIG. 1). Within the LSK population, long-term (LT) HSCs (LSK Flt3⁻CD150⁺CD48⁻) were significantly reduced (FIG. 1), but other subpopulations (i.e., multipotent progenitors (MPPs), including Flt3⁻CD150⁺CD48⁺, Flt3⁻CD150⁻CD48⁺ and Flt3⁻CD150⁻CD48⁻ LSK and lymphoid-primed MPPs (LMPPs) including LSK Flt3⁺CD150⁻) were expanded. T lymphocytes, erythroid precursors and megakaryocytes were decreased. The spleen was enlarged with marked increase in organ cellularity and a significant expansion of LT-HSCs, LMPPs, LSKs, GMPs, CMPs and myeloid cells and reduced T cells. Thus, miR-142 deficit resulted in increase of HSPCs (except for MEPs), decreased homing of LT-HSCs in the BM, evidence of splenic hematopoiesis, and reduced hematopoietic cell output. (Ref 12).

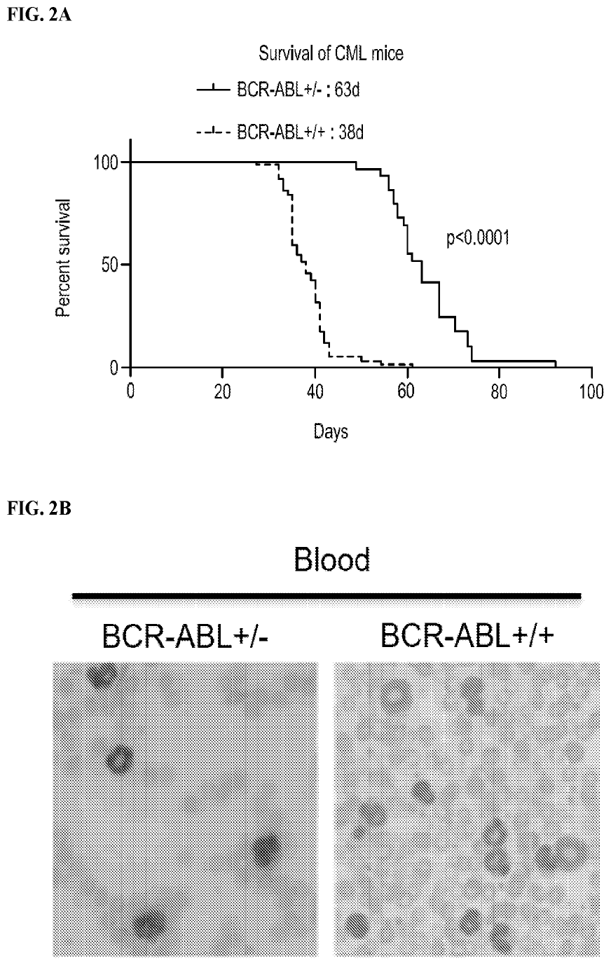

Increase of BCR-ABL dosage does not lead per se to BC in CML mice. The hallmark of CML is the Philadelphia chromosome (Ph) resulting from translocation of chromosomes 9q34 and 22q11, that creates BCR-ABL1, a fusion gene encoding a constitutively active tyrosine kinase. Although tyrosine kinase inhibitors (TKIs) are effective for inducing disease remission and prolonged survival, a subset of CML patients is either intolerant or resistant to this treatment. These patients ultimately progress to a myeloid or lymphoid BC phase if not promptly transplanted. BC CML responds poorly to TKI treatment and is usually fatal. Several studies have shown that transformation of CP to a BC phenotype is a multi-step process. To this end, we have used the SCLtTA/BCR-ABL mouse in a B6 background, a well characterized CP CML model to study the molecular evolution of this disease. This mouse is an inducible transgenic model of CML in which the BCR-ABL gene is expressed under the control of a Tet-regulated 3′ enhancer of the murine stem cell leukemia (SCL) gene, allowing targeted BCR-ABL expression in HSPCs upon tetracycline withdrawal. By back-crossing the original mouse into a B6 background, we have obtained SCLtTA/BCR-ABL double transgenic (i.e., SCLtTA (+/+)/BCR-ABL (+/+), hereafter called BCR-ABL) and single transgenic (i.e., SCLtTA (+/−)/BCR-ABL (+/−)). Both these mice developed and died of CP CML, and neither one developed BC CML (FIG. 2), thereby implying that BCR-ABL dosage is insufficient to induce transformation in this model. (Ref 21, 23-31).

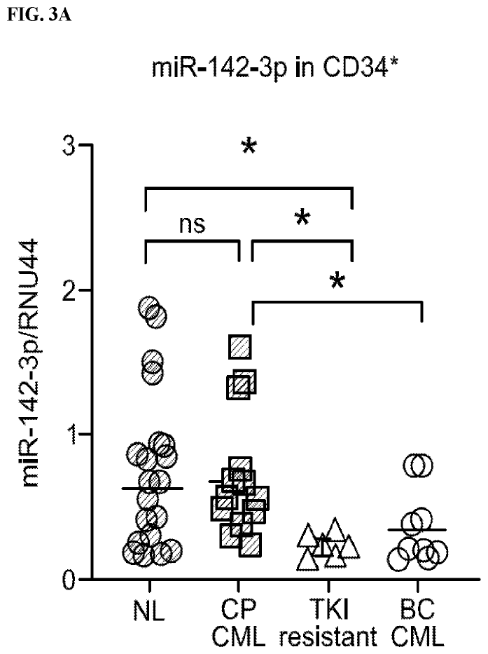

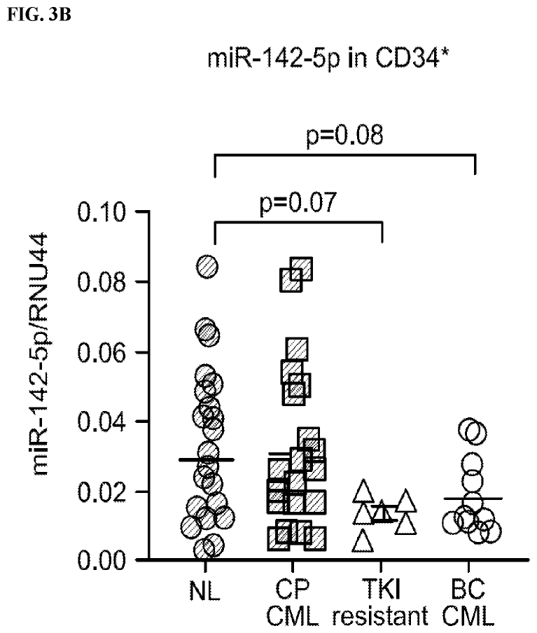

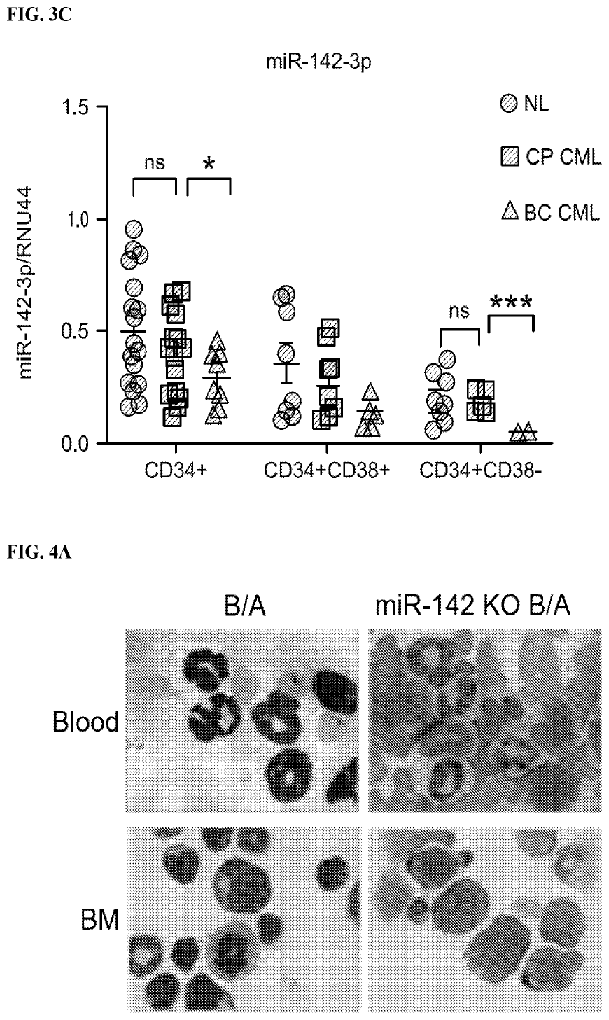

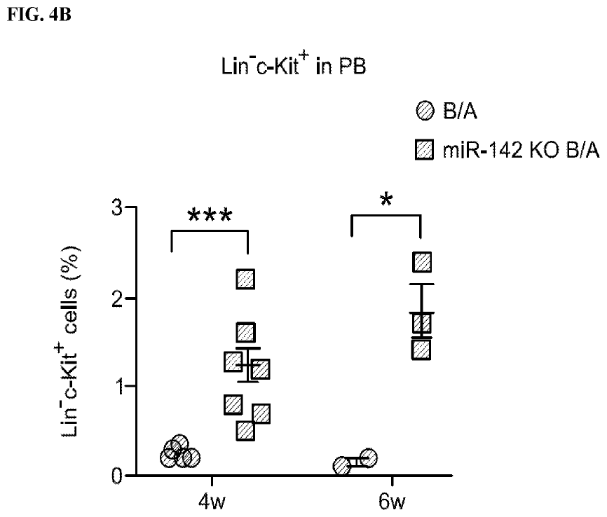

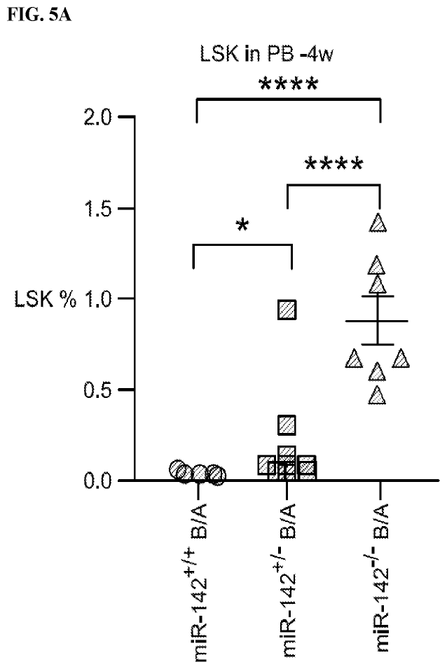

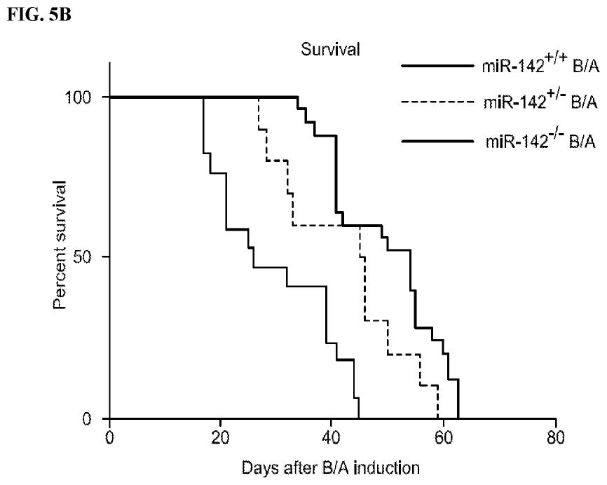

miR-142 deficiency promotes BC in CML mice. Since we noted expansion of HPSCs and decrease of hematopoietic output in the miR-142^{-/-}mouse, and we also observed lower levels of miR-142-3p and miR-142-5p in total MNCs (not shown), CD34⁺ and CD34⁺CD38⁻ cells from patients with BC CML as compared with those from CP CML (FIG. 3), we hypothesized that miR-142 insufficiency could play a role in the transformation of CP into BC CML. To test this hypothesis, we generated miR-142 KO BCR-ABL double transgenic (i.e., miR-142^{-/-}BCR-ABL) mice by crossing miR-142^{-/-} and BCR-ABL mice. We observed circulating leukemic blasts as early as 4 weeks (w) post BCR-ABL induction by tetracycline withdrawal in miR-142^{-/-}BCR-ABL mice. In contrast, no appearance of circulating blasts was observed in miR-142 wt (miR-142^{+/+}) BCR-ABL controls over time, and even when they became moribund, they remained in CP (FIG. 4). An increase in BM leukemic blasts (FIG. 4) and L⁻S⁻K⁺ and LSK cells (>30%, not shown) were demonstrated in miR-142^{-/-}BCR-ABL mice as compared to the miR-142^{+/+}BCR-ABL controls. MiR-142^{-/-}BCR-ABL mice had lower erythrocytes and PLT, larger spleens and significantly shorter survival (FIG. 5, p<0.0001) than the miR-142^{+/+}BCR-ABL controls. Of note, homozygote (homo) miR-142 null (miR-142^{-/-}) BCR-ABL mice had a more rapid progression to BC and a shorter survival (FIG. 5, p=0.003) than the heterozygous (het) ones (i.e., miR-142^{+/-}BCR-ABL), suggesting a dosage effect of the miR-142 deficit.

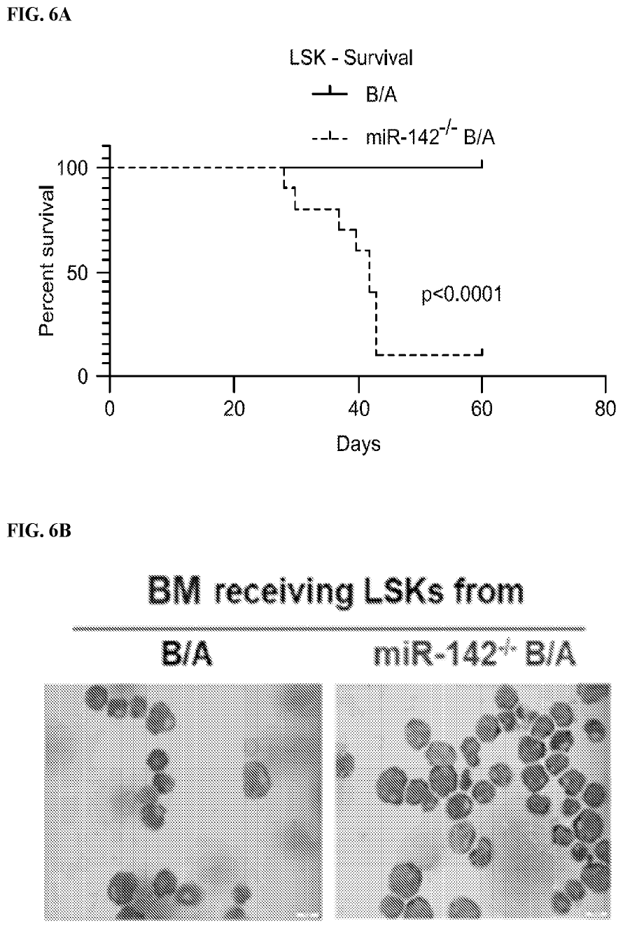

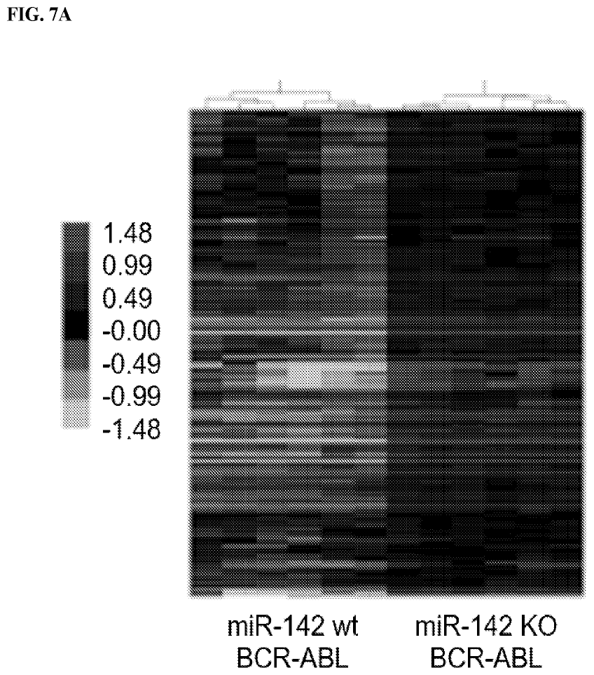

Nevertheless, all the miR-142 null BCR-ABL mice (i.e., both het and homo) eventually transformed to BC, indicating miR-142 deficit as a single event sufficient to initiate BC transformation in the CP CML model. Importantly, all these features were recapitulated in congenic wild-type (wt) recipient mice (CD45.1) transplanted with a relatively small number of BM LSKs (2000 cells/mouse) from diseased miR-142 null BC CML mice (CD45.2) (FIG. 6), supporting that this cell subpopulation is enriched in BC LSCs.

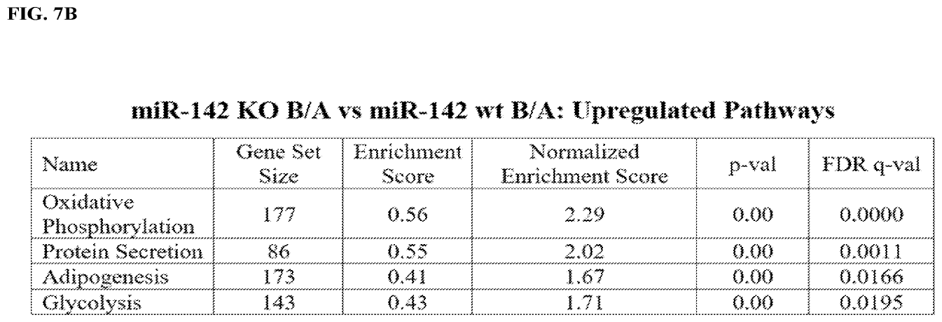

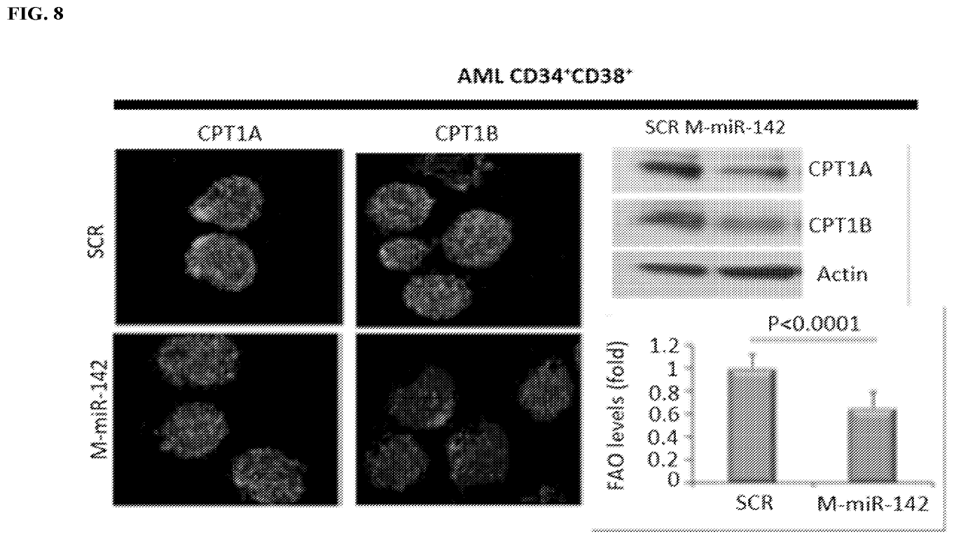

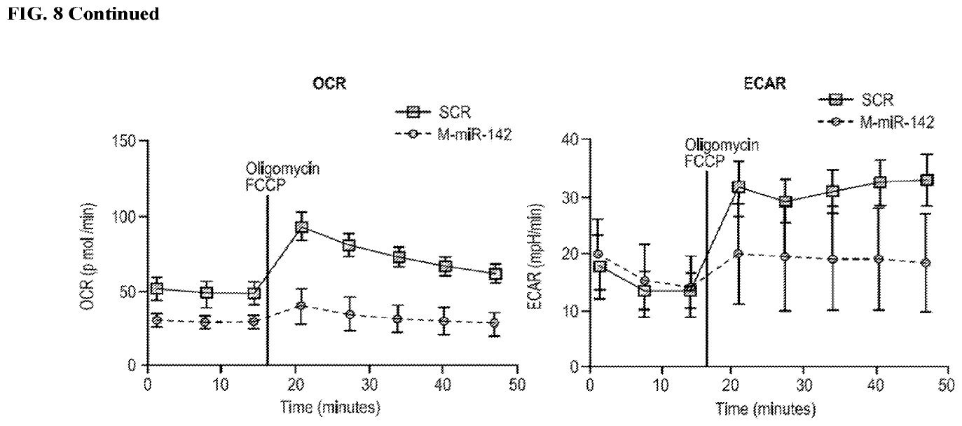

miR-142 deficit deregulates metabolism in LSCs. To gain preliminary information into the role of miR-142 deficiency in BC transformation, we conducted a pilot study of RNA-seq of LSKs from diseased miR-142 null BCR-ABL (BC CML) vs miR-142 wt BCR-ABL (CP CML) mice. We found 504 differentially expressed genes in miR-142 null BCR-ABL LSKs vs miR-142 wt BCR-ABL LSKs (FIG. 7). Gene set enhancement analysis (GSEA) showed that only four pathways were differentially expressed (upregulated); three of them (i.e., oxidative phosphorylation (OxPhos), glycolysis and adipogenesis) regulating cell metabolism (FIG. 7). These results were in line with a recent report by Sun et al showing that miR-142 KO altered the switch from OxPhos to glycolysis in activated dendritic cells by downregulating carnitine palmitoyltransferase-1a (CPT1A). The CPT1 protein family includes three isoforms: CPT1A, CPT1B, and CPT1C that are differentially expressed in various tissues, with CTP1A and 1B being enriched in hematopoietic cells. CPT1 is located in the outer mitochondrial membrane and catalyzes the transfer of the acyl group from a long-chain fatty acyl-CoA to I-carnitine to form palmitoylcarnitine, an essential step for fatty acid oxidation (FAO). In AML, FAO has been shown to play a role in maintaining high levels of OxPhos and low levels of glycolysis, changes that were shown to support the homeostasis of LSCs. Consistent with previous reports, we observed a robust expression of CPT1A/B and a high oxygen consumption rate (OCR) indicative of high OxPhos and a low extracellular acidification rate (ECAR) indicative of low glycolysis in human LSC-enriched AML CD34⁺CD38⁻ cells as compared with normal CD34⁺CD38⁻ HSCs (not shown). Treatment with CpG-M-miR-142 (2 μM for 24 h, FIG. 15) resulted in CPT1A/B downregulation followed by reduction of FAO and OCR (OxPhos), while ECAR (glycolysis) remained unchanged (FIG. 8, p>0.05), as compared with CpG-scrambled RNA (SCR) control. While the impact of miR-142 deficit on the cell metabolism is intriguing, the mechanisms through which these miR-142-dependent changes contribute to the transformation of clonal HSCs of CP CML mice into LSCs of BC CML are unknown. (Ref 33-39).

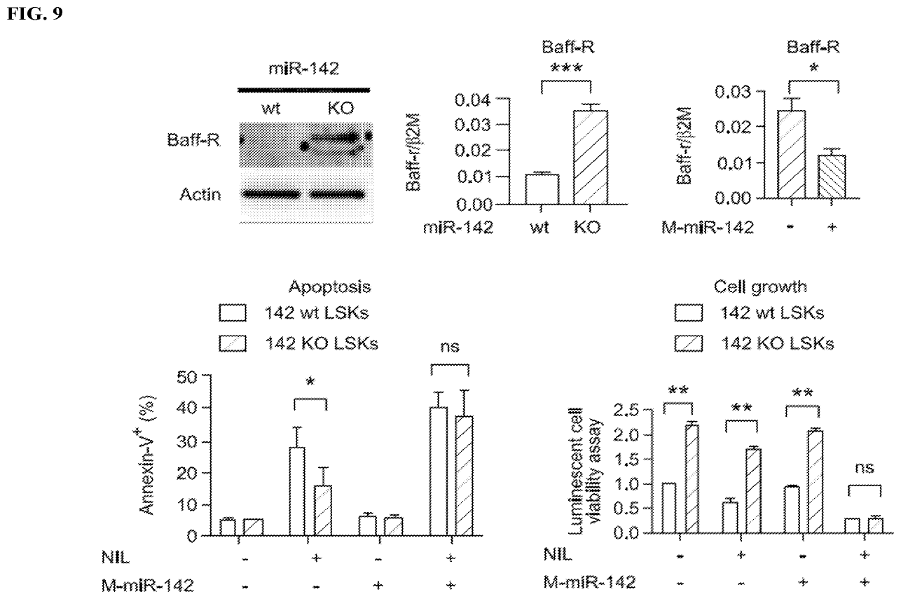

miR-142 deficit promotes TKI resistance in BCR-ABL cells. In addition to the observation that miR-142 deficit leads to BC CML in the mouse, we also observed that miR-142 levels were significantly lower in patients with BC CML and in TKI resistant patients with CP CML compared to TKI sensitive patients with CP CML (FIG. 3). This leads us to hypothesize that reduced expression of miR-142 associates with TKI resistance. To prove this point, we selected LSKs from miR-142 null and miR-142 wt BCR-ABL mice at 2 w post BCR-ABL induction, and exposed them to the TKI nilotinib (NIL; 2 μM) or vehicle for 72 hours (h). We observed higher cell growth and lower apoptosis in miR-142 null LSKs than miR-142 wt LSKs (FIG. 9, lower panels). We showed that these differences could be attenuated by rescuing the miR-142 deficit. LSKs from diseased miR-142 null and miR-142 wt BCR-ABL mice were treated with CpG-M-miR-142 (2 μM) or SCR±NIL (2 μM) for 72 h. CpG-M-miR-142 alone reduced the expression of miR-142 targets (e.g., Baff-R) in LSKs from miR-142 null BCR-ABL mice (FIG. 9, right upper panel). CpG-M-miR-142 plus NIL significantly increased apoptosis and reduced cell growth in miR-142 null BCR-ABL LSKs as compared with SCR+NIL (FIG. 9, lower panels). Of note, combination of CpG-M-miR-142 and NIL also increased apoptosis and reduced cell growth in miR-142 wt BCR-ABL LSKs (FIG. 9, lower panels). Similar results were observed using human CD34⁺CD38⁻ primitive cells from CML patients in CP (miR-142^{hi}g^{h}) or BC (miR-142^{low}) (not shown).

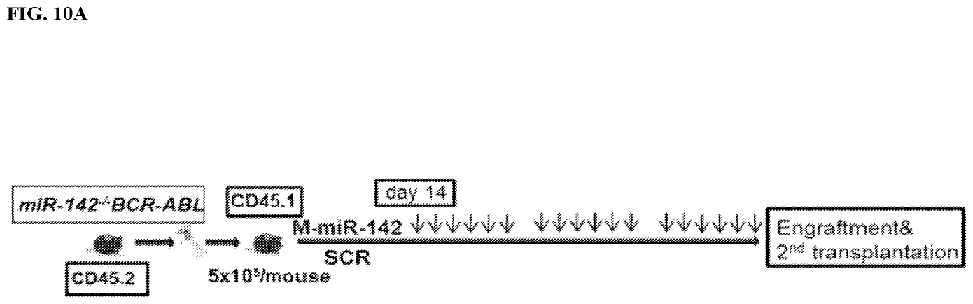

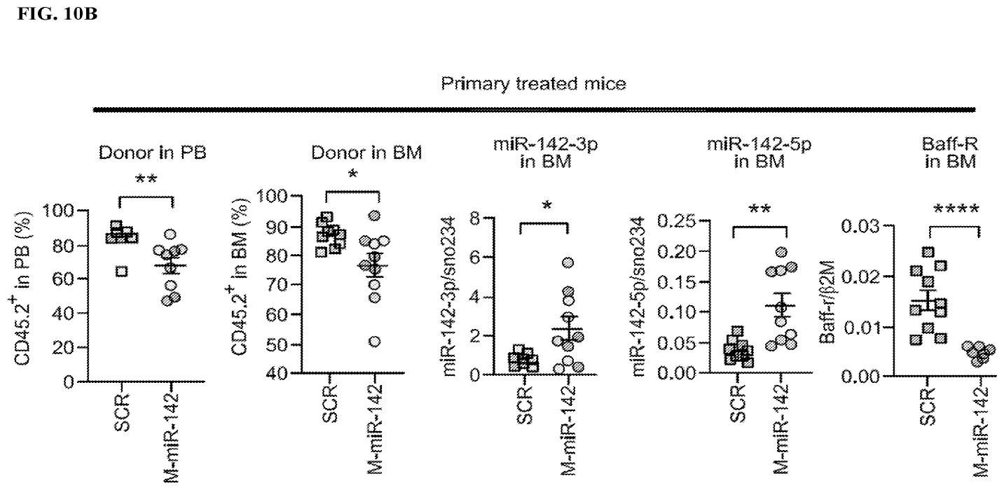

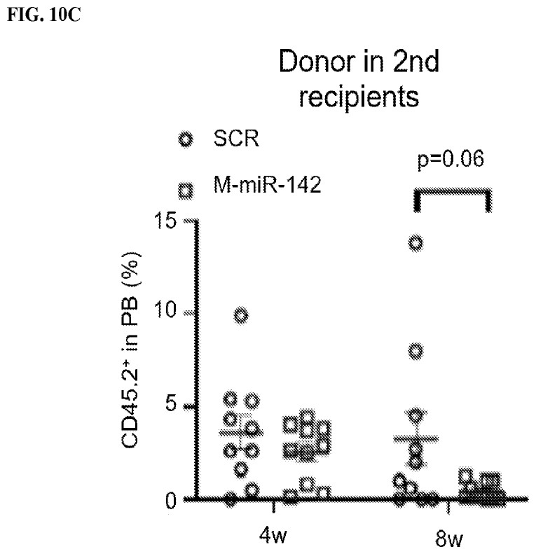

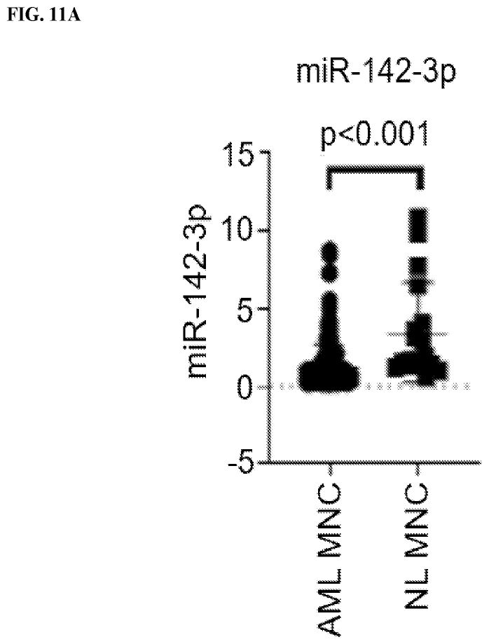

In vivo activity of CpG-M-miR-142. To obtain evidence of activity of CpG-M-miR-142 in vivo, we then engrafted congenic recipient mice (CD45.1) with BM cells from the miR-142 null BCR-ABL BC CML mice (CD45.2) and treated them with CpG-M-miR-142 (20 mg/kg/day, IV) or SCR for 3 w. We observed reduced blasts in PB and BM (FIG. 10) and smaller spleens (not shown) in CpG-M-miR-142-treated mice compared with SCR-treated controls as early as 3 w. We also detected a significantly increased expression of both miR-142-3p- and -5p and reduced expression of the miR-142 target Baff-R in BM cells from CpG-M-miR-142-treated mice vs SCR-treated mice (FIG. 10). Secondary (2^{nd}) recipients transplanted with BM from CpG-M-miR-142-treated donors showed reduced PB engraftment and disease burden at 8 w compared with recipients transplanted with BM from SCR-treated donors (FIG. 10, right), indicating that CpG-M-miR-142 decreased LSC burden.

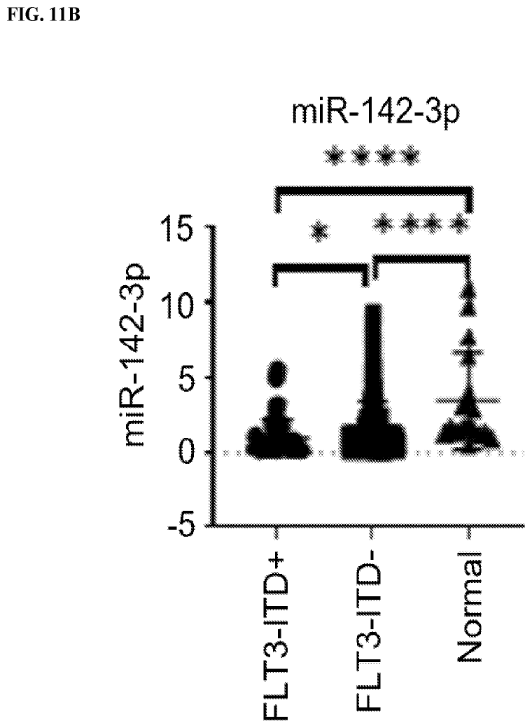

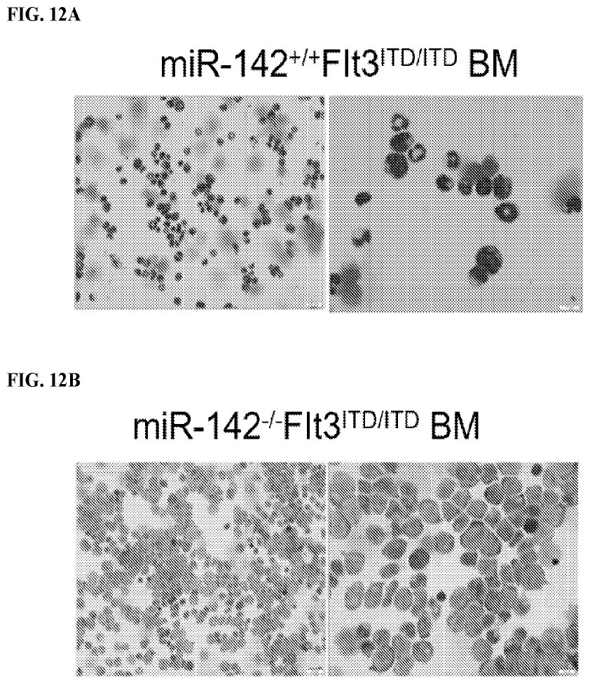

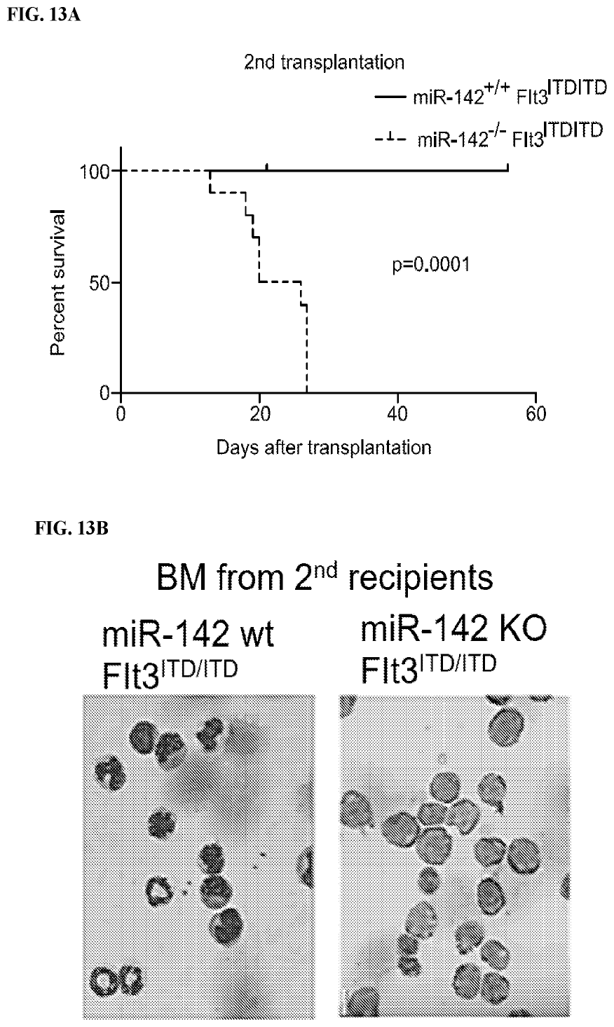

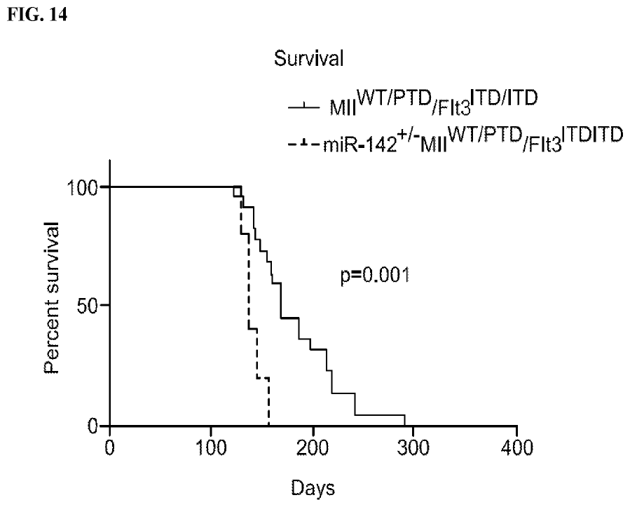

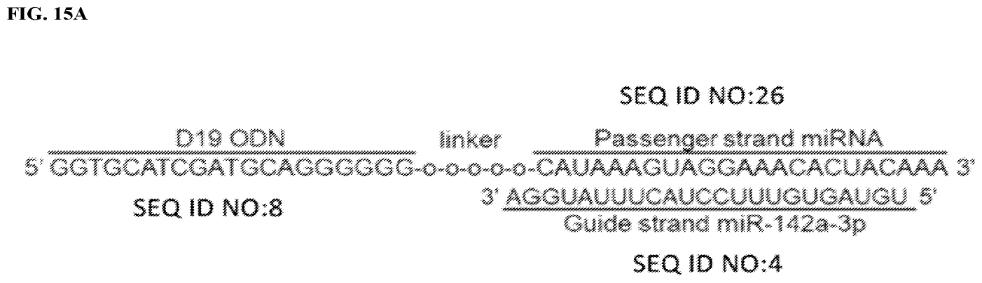

miR-142 deficit promotes transformation of FLT3^{IT)/IT)} MPN into AML. Mutations of the FLT3 gene are found in approximately 30% of newly diagnosed AML cases and occur as either internal tandem duplication (ITDs; about 25%) or tyrosine kinase domain (TKD) point mutations (7-10%). Both FLT3-ITD and FLT3-TKD mutations constitutively activate FLT3 kinase activity, resulting in proliferation and survival of AML cells. Although FLT3 mutations are often associated with other recurrent mutations (e.g., NPM1, IDH1 and IDH2), they may also present as the only detectable mutations in AML patients. Interestingly, we noted that FLT3-ITD+ AML patients present with lower miR-142 levels compared with FLT3-ITD-AML and normal donors (FIG. 11). The Flt3-ITD knock-in (Flt3^{ITD/ITD}) mouse develops only a MPN-like phenotype, and not AML. Thus, we hypothesize that, similar to transformation of CP CML into BC, miR-142 deficit could also transform murine Flt3-ITD+ MPN into sAML. To test this hypothesis, we crossed the miR-142−/− and Flt3^{ITD/ITD} strains to generate miR-142^{-/-}Flt3^{ITD/ITD} progeny. MiR-142^{+/+}Flt3^{ITD/ITD} mice developed only MPN (median survival: 623 days), without evidence of overt AML. In contrast miR-142^{-/-}Flt3^{ITD/ITD} mice progressed to AML, with a rapid and significant increase in WBC counts (>100 k/μl at the end stage) and circulating (>5%) and BM (>20%) leukemic blasts and a significantly reduced survival (p=0.0004, FIG. 12). Importantly, 2^{nd} transplantation of BM from diseased miR-142^{-/-}Flt3^{ITD/ITD} mice yielded an aggressive AML-like phenotype with a median survival of only 23 days in recipient mice (FIG. 13). Of note, when we crossed miR-142 KO mice with Mll^{PTD/wt}/Flt3^{ITD/ITD} mice, which already have an established AML phenotype, all miR-142 homo null (miR-142^{-/-})Mll^{PTD/wt}/Flt3^{ITD/ITD} offspring died of aggressive AML-like disease within only 4 weeks from birth. The miR-142 null het (miR-142^{+/-}) Mll^{PTD/wt}/Flt3^{ITD/ITD} mice also showed a more aggressive AML phenotype and a shorter survival than the miR-142 wt Mll^{PTD/wt}/Flt3^{ITD/ITD} mice (p=0.001, FIG. 14). These aggressive features were recapitulated in 2nd transplant experiments, suggesting that loss of miR-142 increases LSC disease-initiating capacity. Interestingly, similar to the CML model, LSKs from miR-142 null Mll^{PTD/wt}/Flt3^{ITD/ITD} mice were more resistant to TKI than LSKs from miR-142 wt Mll^{PTD/wt}/Flt3^{ITD/ITD} controls (not shown). (Ref 40, 41).

Summary. The results demonstrate a key role of miR-142 deficit in the transformation of MPD into aggressive AML-like diseases (BC CML or sAML). While the mechanisms through which transformation occurs remain to be elucidated, the inventors observed enrichment of metabolic pathways in LSK cells from miR-142 null mice, indicating a deregulation of basic mechanisms of cell homeostasis. miR-142 deficit also confers TKI-resistance that can be rescued with CpG-M-miR-142, also capable of decreasing LSC burden in vivo.

1. A compound of Formula (I):
R¹-L¹-R² (I);
wherein:
R¹ is a CpG oligodeoxynucleotide (ODN);
L¹ is a linking group;
R² is a hybridized nucleic acid sequence comprising:
(i) a miR-142 passenger strand sequence comprising SEQ ID NO:26 hybridized to a miR-142 guide strand sequence comprising SEQ ID NO:4;
(ii) a miR-142 passenger strand sequence comprising SEQ ID NO:27 hybridized to a miR-142 guide strand sequence comprising SEQ ID NO:28;
(iii) a miR-142 passenger strand sequence comprising SEQ ID NO:1 hybridized to a miR-142 guide strand sequence comprising SEQ ID NO:4;
(iv) a miR-142 passenger strand sequence comprising SEQ ID NO:2 hybridized to a miR-142 guide strand sequence comprising SEQ ID NO:4; or
(v) a miR-142 passenger strand sequence comprising SEQ ID NO:3 hybridized to a miR-142 guide strand sequence comprising SEQ ID NO:5.

2-5. (canceled)

6. The compound of claim 1, wherein one or more nucleotides in the miR-142 passenger strand sequence are modified with 2′-O-Methyl, 2′-Fluoro, a phosphorothioate moiety, or a combination thereof.

7. The compound of claim 1, wherein one or more nucleotides in the miR-142 guide strand sequence are modified with 2′-O-Methyl, 2′-Fluoro, a phosphorothioate moiety, or a combination thereof.

8. (canceled)

9. The compound of claim 1, wherein the 3′ end of R¹ is covalently bonded to L¹ and the 5′ end of the miR-142 passenger strand sequence is covalently bonded to L¹.

10. The compound of claim 1, wherein the miR-142 passenger strand sequence is covalently bonded to the linking group; and wherein the miR-142 guide strand sequence is hybridized to the miR-142 passenger strand sequence.

11-12. (canceled)

13. The compound of claim 1, wherein the R¹ comprises a CpG-A ODN, a CpG-B ODN, or a CpG-C ODN.

14. (canceled)

15. The compound of claim 1, wherein R¹ comprises the sequence of any one of SEQ ID NOS:6-25.

16. The compound of claim 1, wherein one or more nucleotides in the CpG oligodeoxynucleotide (ODN) of R¹ are modified with a phosphorothioate moiety.

17. The compound of claim 1, wherein L¹ is a bond, a nucleic acid sequence, substituted or unsubstituted alkylene, a substituted or unsubstituted heteroalkylene, or a combination of two or more thereof.

18. The compound of claim 17, wherein L¹ is a substituted 6 to 48 membered heteroalkylene; wherein the heteroalkylene comprises an oxygen atom, a phosphorous atom, or a combination thereof, and wherein the substituents are independently selected from the group consisting of ═O, —OH, and —O⁻.

19. The compound of claim 17, wherein L¹ is: 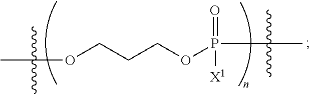
wherein X¹ is independently —OH or —O⁻, and n is an integer from 1 to 8.

20-22. (canceled)

23. A pharmaceutical composition comprising the compound of claim 1 and a pharmaceutically acceptable excipient.

24. A method of treating myeloid leukemia in a patient in need thereof, the method comprising administering to the patient an effective amount of the compound of any one of claim 1.

25. (canceled)

26. A method of treating myeloid leukemia in a patient in need thereof, the method comprising:
(i) detecting reduced miR-142 levels relative to a control in a biological sample obtained from the patient;
(ii) administering to the patient an effective amount of the compound of claim 1.

27. A method of treating myeloid leukemia in a patient in need thereof, the method comprising:
(i) measuring miR-142 levels in a biological sample obtained from the patient;
(ii) identifying the patient as having myeloid leukemia when the miR-142 levels are reduced relative to a control; and
(iii) administering to the patient identified as having myeloid leukemia based on the reduced miR-142 levels an effective amount of the compound of claim 1.

28-42. (canceled)

43. A method of treating myelodysplastic syndrome, myeloproliferative neoplasm, or myelodysplastic syndrome/myeloproliferative neoplasm overlap syndrome in a patient in need thereof, the method comprising administering to the patient an effective amount of the compound of claim 1.

44. (canceled)

45. A method of treating myelodysplastic syndrome, myeloproliferative neoplasm, or myelodysplastic syndrome/myeloproliferative neoplasm overlap syndrome in a patient in need thereof, the method comprising:
(i) detecting reduced miR-142 levels relative to a control in a biological sample obtained from the patient; and
(ii) administering to the patient an effective amount of the compound of claim 1.

46. A method of treating myelodysplastic syndrome, myeloproliferative neoplasm, or myelodysplastic syndrome/myeloproliferative neoplasm overlap syndrome in a patient in need thereof, the method comprising:
(i) measuring miR-142 levels in a biological sample obtained from the patient;
(ii) identifying the patient as having myelodysplastic syndrome, myeloproliferative neoplasm, or myelodysplastic syndrome/myeloproliferative neoplasm overlap syndrome when the miR-142 levels are reduced relative to a control; and
(iii) administering to the patient identified as having myelodysplastic syndrome, myeloproliferative neoplasm, or myelodysplastic syndrome/myeloproliferative neoplasm overlap syndrome based on the reduced miR-142 levels an effective amount of the compound of claim 1.

47-50. (canceled)

51. A method of preventing or delaying the progression of an antecedent clonal hematopoietic disorder in a patient in need thereof, the method comprising administering to the patient an effective amount of the compound of claim 1; wherein the patient has the antecedent clonal hematopoietic disorder.

52-60. (canceled)

61. A method of treating aplastic anemia in a patient in need thereof, the method comprising administering to the patient an effective amount of the compound of claim 1.

62-64. (canceled)
